(19) United States
(12) Patent Application Publication
(10) Pub. No.: US 2009/0143411 A1
(43) Pub. Date: Jun. 4, 2009

(54) Thiophens and Their Use as Anti-Tumor Agents

(75) Inventors: John Ward, Redwood City, CA (US); Rama Jain, Fremont, CA (US); Donald R. James, El Sobrante, CA (US); Herman J. Verheij, Papendrecht (NL); Jan C.C. Schultz, The Hague (NL)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: COMPASS PHARMACEUTICALS LLC, Union, NJ (US)

(21) Appl. No.: 11/664,544

(22) PCT Filed: Oct. 18, 2005
(86) PCT No.: PCT/US05/37307

§ 371 Date: Sep. 2, 2008

Related U.S. Application Data
(60) Provisional application No. 60/620,615, filed on Oct. 20, 2004.

(51) Int. Cl. A61K 31/519; C07D 333/10; C07D 491/04; A61P 35/00; A61K 31/381

(52) U.S. Cl. 514/258.1; 549/57; 544/278; 514/443

(57) ABSTRACT

The present invention provides novel compounds and pharmaceutical compositions thereof, as well as methods for using the compounds and pharmaceutical compositions for treating tumors. Examples of specific tumor types that the compounds may be used to treat include, but are not limited to sarcomas, melanomas, neuroblastomas, carcinomas (including but not limited to lung, renal cell, ovarian, liver, bladder, and pancreatic carcinomas), and mesotheliomas.

### CROSS REFERENCE

This application claims priority to U.S. Provisional Patent Application Ser. No. 60/620,615 filed Oct. 20, 2004, which is incorporated by reference herein in its entirety.

### BACKGROUND OF THE INVENTION

Approximately twenty percent of deaths from all causes in the United States are cancer-related. Although chemotherapy is a principal means of cancer treatment, the rate at which effective new drugs have become available for use in cancer chemotherapy has not increased (Horowitz et al., Journal of Clinical Oncology, Vol. 6, No. 2, pp. 308-314 (1988)). Despite many years of promising new therapies, cancer remains a major cause of morbidity and mortality (Bailar et al., N. Engl. J. Med. 336:1569-1574, 1997). Accordingly, there is a substantial need for new drugs that are effective in inhibiting the growth of tumors.

### SUMMARY OF THE INVENTION

The present invention provides novel compounds and pharmaceutical compositions thereof, as well as methods for using the compounds and pharmaceutical compositions for treating tumors. Examples of specific tumor types that the compounds may be used to treat include, but are not limited to sarcomas, melanomas, neuroblastomas, carcinomas (including but not limited to lung, renal cell, ovarian, liver, bladder, and pancreatic carcinomas), and mesotheliomas.

In one aspect, the present invention provides novel compounds according the general formula I:

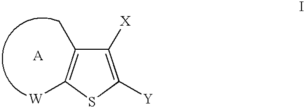

wherein
W is a carbon atom or nitrogen atom;
Y is —NR¹R² and X is —NR^{5′}R⁶, —C(O)NR⁵R⁶ or —C(O)OR⁸, or
Y is —C(O)NR¹R⁵ and X is —NR^{5′}R⁶, or
Y is NO₂ and X is CH═CH—C(O)OR′;
R¹ and R′ are independently selected from hydrogen or lower alkyl;
R² is selected from hydrogen, —C(O)R¹⁰, —C(O)CH₂OC(O)CH₃, —SO₂R¹⁰;
R⁶ is hydrogen, lower alkyl, —SO₂R^{10′}, or
R² and R⁶, or R² and R⁸, both when Y is —NR¹R², together with respective atoms to which they are attached are connected to form a 6-10 membered ring C, which can include double bond and/or a fused bicyclic ring, wherein Z is —N(R⁵)— or —O—,

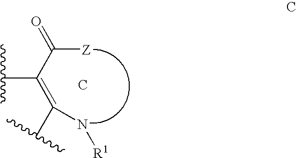

which can be optionally substituted with one or two groups selected from lower alkyl, lower alkenyl, lower alkynyl, trifluoromethyl, difluoromethyl, chlorodifluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷;
R⁵, R^{5′} and R^{5″} are independently hydrogen, or
R⁵, R^{5′} and R^{5″} are independently lower alkyl optionally substituted with one to five groups selected from halo, hydroxyl, lower alkoxy, lower alkenyl and lower alkynyl, or
R⁵, R^{5′} and R^{5″} are independently selected from cycloalkyl, cycloalkylalkyl, heterocyclyl, heterocyclylalkyl, aryl, arylalkyl, heteroaryl or heteroarylalkyl, the ring portion of each is optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷, or
R⁵ and R⁶ together with the nitrogen atom to which they are attached form a 5-7 membered heterocyclic ring optionally substituted with one to three groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷;
R⁷ and R^{7′} are independently selected from hydrogen, lower alkyl, aryl, heteroaryl, arylalkyl, heteroarylalkyl, —C(O)R′ or —C(O)OR′;
R⁸ and R′ are independently selected from hydrogen, lower alkyl and lower alkenyl;
R¹⁰ and R^{10′} are independently selected from —NHR¹⁵, —C(O)OR′, or
R¹⁰ and R^{10′} are independently lower alkyl optionally substituted with one to eight groups selected from halo, hydroxyl, lower alkenyl, lower alkynyl, lower alkoxy, —NR⁸R′, or
R¹⁰ and R^{10′} are independently selected from cycloalkyl, cycloalkylalkyl, heterocyclyl, heterocyclylalkyl, aryl, arylalkyl, heteroaryl or heteroarylalkyl, the ring portion of each is optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷, or
   aryl or heteroaryl optionally substituted with one or two groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷;
R¹⁵ is lower alkyl, aryl or heteroaryl; and
the A ring represents a 5-14 membered aryl, heteroaryl, cycloalkyl or heterocycloalkyl ring, each of which is optionally substituted with one to three groups independently selected from lower alkyl, halo, oxo, nitro, —CN, lower alkenyl, lower alkynyl, trifluoromethyl, —C(O)R⁷, C(O)OR′, —C(O)NR^{7′}R⁷ and —NHC(O)R⁷, or
   aryl or heteroaryl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷.

In another aspect the present invention provides pharmaceutical compositions, comprising one or more compounds according to the invention, and a pharmaceutically acceptable carrier.

In another aspect, the present invention provides methods for treating a subject with a tumor, comprising administering to the subject an amount effective of a compound according to formula II:

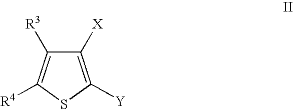

wherein
Y is —NR¹R² and X is —NR^{5′}R⁶, —C(O)NR⁵R⁶ or —C(O)OR⁶, or
Y is —C(O)NR¹R⁵ and X is —NR^{5′}R⁶, or
Y is NO₂ and X is CH═CH—C(O)OR′;
R¹ and R′ are independently selected from hydrogen or lower alkyl;
R² is selected from hydrogen, —C(O)R¹⁰, —C(O)CH₂OC(O)CH₃, —SO₂R¹⁰;
R⁶ is hydrogen, lower alkyl, —SO₂R^{10′}, or
R² and R⁶, or R² and R⁸, both when Y is —NR¹R², together with respective nitrogen atoms to which they are attached are connected to form a 6-10 membered ring C, which can include a double bond and/or a fused bicyclic ring, wherein Z is —N(R⁵)— or —O—,

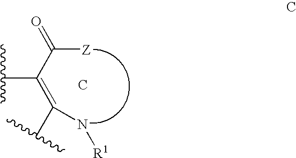

which can be optionally substituted with one or two groups selected from lower alkyl, lower alkenyl, lower alkynyl, trifluoromethyl, difluoromethyl, chlorodifluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷;
R³ and R⁴ are independently selected from hydrogen, lower alkyl, lower alkenyl, lower alkynyl, lower alkoxy, halo, —C(O)OR′, —C(O)NHR^{5″}, or
R³ is aryl optionally substituted with lower alkyl, lower alkoxy or halo, or
R³ and R⁴ together with the carbon atoms to which they are attached form a 5-14 membered aryl, heteroaryl, cycloalkyl or heterocycloalkyl ring, each of which is optionally substituted with one to three groups independently selected from lower alkyl, halo, oxo, nitro, —CN, lower alkenyl, lower alkynyl, trifluoromethyl, —C(O)R⁷, C(O)OR′, —C(O)NR^{7′}R⁷ and —NHC(O)R⁷, or
   aryl or heteroaryl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷;
R⁵, R^{5′} and R^{5″} are independently hydrogen, or
R⁵, R^{5′} and R^{5″} are independently lower alkyl optionally substituted with one to five groups selected from halo, hydroxyl, lower alkoxy, lower alkenyl and lower alkynyl, or
R⁵, R^{5′} and R^{5″} are independently selected from cycloalkyl, cycloalkylalkyl, heterocyclyl, heterocyclylalkyl, aryl, arylalkyl, heteroaryl or heteroarylalkyl, the ring portion of each is optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷, or
R⁵ and R⁶ together with the nitrogen atom to which they are attached form a 5-7 membered heterocyclic ring optionally substituted with one to three groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷;
R⁷ and R^{7′} are independently selected from hydrogen, lower alkyl, aryl, heteroaryl, arylalkyl, heteroarylalkyl, —C(O)R′ or —C(O)OR′;
R⁸ and R′ are independently selected from hydrogen, lower alkyl and lower alkenyl;
R¹⁰ and R^{10′} are independently selected from —NHR¹⁵, —C(O)OR′, or
R¹⁰ and R^{10′} are independently lower alkyl optionally substituted with one to eight groups selected from halo, hydroxyl, lower alkenyl, lower alkynyl, lower alkoxy, —NR⁸R′, or
R¹⁰ and R^{10′} are independently selected from cycloalkyl, cycloalkylalkyl, heterocyclyl, heterocyclylalkyl, aryl, arylalkyl, heteroaryl or heteroarylalkyl, the ring portion of each is optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷, or
   aryl or heteroaryl optionally substituted with one or two groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷; and
R¹⁵ is lower alkyl, aryl or heteroaryl.

### BRIEF DESCRIPTION OF THE FIGURES

FIG. 1 is a table showing anti-tumor activity of representative compounds of the invention.

### DETAILED DESCRIPTION OF THE INVENTION

All references cited herein are incorporated by reference in their entirety.

In one aspect, the present invention provides novel compounds according the general formula I:

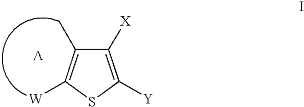

wherein
W is a carbon or nitrogen atom;
Y is —NR¹R² and X is —NR^{5′}R⁶, —C(O)NR⁵R⁶ or —C(O)OR⁶, or
Y is —C(O)NR¹R⁵ and X is —NR^{5′}R⁶, or
Y is NO₂ and X is CH═CH—C(O)OR′;
R¹ and R′ are independently selected from hydrogen or lower alkyl;
R² is selected from hydrogen, —C(O)R¹⁰, —C(O)CH₂OC(O)CH₃, —SO₂R¹⁰;
R⁶ is hydrogen, lower alkyl, —SO₂R^{10′}, or
R² and R⁶, or R² and R⁸, both when Y is —NR¹R², together with respective nitrogen atoms to which they are attached are connected to form a 6-10 membered ring C, which can include a double bond and/or a fused bicyclic ring, wherein Z is —N(R⁵)— or —O—,

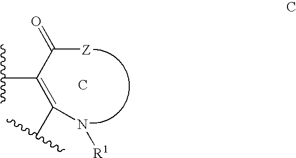

which can be optionally substituted with one or two groups selected from lower alkyl, lower alkenyl, lower alkynyl, trifluoromethyl, difluoromethyl, chlorodifluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷;
R⁵, R^{5′} and R^{5″} are independently hydrogen, or
R⁵, R^{5′} and R^{5″} are independently lower alkyl optionally substituted with one to five groups selected from halo, hydroxyl, lower alkoxy, lower alkenyl and lower alkynyl, or
R⁵, R^{5′} and R^{5″} are independently selected from cycloalkyl, cycloalkylalkyl, heterocyclyl, heterocyclylalkyl, aryl, arylalkyl, heteroaryl or heteroarylalkyl, the ring portion of each is optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷, or
R⁵ and R⁶ together with the nitrogen atom to which they are attached form a 5-7 membered heterocyclic ring optionally substituted with one to three groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷;
R⁷ and R^{7′} are independently selected from hydrogen, lower alkyl, aryl, heteroaryl, arylalkyl, heteroarylalkyl, —C(O)R′ or —C(O)OR⁷;
R⁸ and R′ are independently selected from hydrogen, lower alkyl and lower alkenyl;
R¹⁰ and R^{10′} are independently selected from —NHR¹⁵, —C(O)OR′, or
R¹⁰ and R^{10′} are independently lower alkyl optionally substituted with one to eight groups selected from halo, hydroxyl, lower alkenyl, lower alkynyl, lower alkoxy, —NR⁸R′, or
R¹⁰ and R^{10′} are independently selected from cycloalkyl, cycloalkylalkyl, heterocyclyl, heterocyclylalkyl, aryl, arylalkyl, heteroaryl or heteroarylalkyl, the ring portion of each is optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷, or
   aryl or heteroaryl optionally substituted with one or two groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷;
R¹⁵ is lower alkyl, aryl or heteroaryl; and
the A ring represents a 5-14 membered aryl, heteroaryl, cycloalkyl or heterocycloalkyl ring, each of which is optionally substituted with one to three groups independently selected from lower alkyl, halo, oxo, nitro, —CN, lower alkenyl, lower alkynyl, trifluoromethyl, —C(O)R⁷, C(O)OR′, —C(O)NR^{7′}R⁷ and —NHC(O)R⁷, or
   aryl or heteroaryl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷;

and pharmaceutically acceptable derivatives thereof.

In an embodiment, the invention relates to compounds of formula I wherein the A ring is a 6-10 membered aryl, heteroaryl, cycloalkyl or heterocycloalkyl ring, each of which is optionally substituted with one to three groups independently selected from lower alkyl, halo, oxo, nitro, —CN, lower alkenyl, lower alkynyl, trifluoromethyl, —C(O)R⁷, C(O)OR′, —C(O)NR^{7′}R⁷ and —NHC(O)R⁷; or aryl or heteroaryl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷.

The invention also relates to compounds of formula Ia:

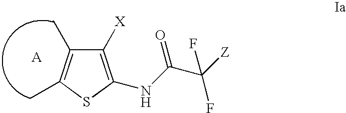

wherein the A ring and X are defined above for formula I and Z is hydrogen, halo or lower alkyl substituted with from between 2 to 6 halo.

In an embodiment, the invention relates to compounds of formula Ia wherein Z is hydrogen, chloro, fluoro or —CF₂—CF₂—CF₃.

In yet another embodiment, the invention relates to compounds of formula Ia wherein X is —C(O)NR⁵R⁶.

In still another embodiment, the invention relates to compounds of formula Ia wherein R⁶ is hydrogen and R⁵ is aryl or heteroaryl optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷. In a preferred embodiment, R⁵ is phenyl or pyridin-2-yl optionally substituted with one to two groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷.

In another embodiment, the invention relates to compounds of formula Ia wherein the A ring is a 6-10 membered aryl, heteroaryl, cycloalkyl or heterocycloalkyl ring, each of which is optionally substituted with one to three groups independently selected from lower alkyl, halo, oxo, nitro, —CN, lower alkenyl, lower alkynyl, trifluoromethyl, —C(O)R⁷, C(O)OR′, —C(O)NR^{7′}R⁷ and —NHC(O)R⁷; or aryl or heteroaryl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷.

The invention also relates to compounds of formula Ib:

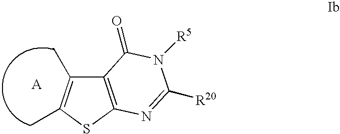

wherein the A ring and R⁵ are as defined above for formula I and R²⁰ is selected from lower alkyl, lower alkenyl, lower alkynyl, trifluoromethyl, difluoromethyl, chlorodifluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷.

In an embodiment, the invention relates to compounds of formula Ib wherein R²⁰ is trifluoromethyl or chlorodifluoromethyl.

In yet another embodiment, the invention relates to compounds of formula Ib wherein R⁵ is aryl or heteroaryl optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷. In a preferred embodiment, R⁵ is phenyl or pyridin-2-yl optionally substituted with one to two groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷.

In another embodiment, the invention relates to compounds of formula Ib wherein the A ring is a 6-10 membered aryl, heteroaryl, cycloalkyl or heterocycloalkyl ring, each of which is optionally substituted with one to three groups independently selected from lower alkyl, halo, oxo, nitro, —CN, lower alkenyl, lower alkynyl, trifluoromethyl, —C(O)R⁷, C(O)OR′, —C(O)NR^{7′}R⁷ and —NHC(O)R⁷; or aryl or heteroaryl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷.

The invention also relates to compounds of formula Ic:

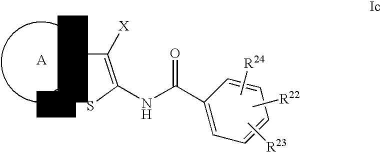

wherein the A ring and X are as defined above for formula I and R²², R²³ and R²⁴ are independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷; or aryl or heteroaryl optionally substituted with one or two groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷.

In an embodiment, the invention relates to compounds of formula Ic wherein X is —C(O)NR⁵R⁶.

In still another embodiment, the invention relates to compounds of formula Ic wherein R⁶ is hydrogen and R⁵ is aryl or heteroaryl optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷. In a preferred embodiment, R⁵ is phenyl or pyridin-2-yl optionally substituted with one to two groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷.

In yet another embodiment, the invention relates to compounds of formula Ic wherein the A ring is a 6-10 membered aryl, heteroaryl, cycloalkyl or heterocycloalkyl ring, each of which is optionally substituted with one to three groups independently selected from lower alkyl, halo, oxo, nitro, —CN, lower alkenyl, lower alkynyl, trifluoromethyl, —C(O)R⁷, C(O)OR′, —C(O)NR^{7′}R⁷ and —NHC(O)R⁷; or aryl or heteroaryl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷.

In another embodiment, the invention relates to compounds of formula Ic wherein R²² is hydrogen, lower alkyl or lower alkoxy and R²³ is selected from hydrogen, halo, —SR′, lower alkoxy and lower alkyl.

The invention also relates to compounds of formula Id:

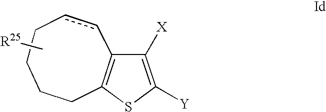

wherein X and Y are as defined above for formula I, — is an optional bond, and R²⁵ is selected from lower alkyl, halo, oxo, nitro, —CN, lower alkenyl, lower alkynyl, trifluoromethyl, —C(O)R⁷, C(O)OR′, —C(O)NR^{7′}R⁷ and —NHC(O)R⁷; or aryl or heteroaryl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷.

In an embodiment, the invention relates to compounds of formula Id wherein X is —C(O)NR⁵R⁶.

In still another embodiment, the invention relates to compounds of formula Id wherein R⁶ is hydrogen and R⁵ is aryl or heteroaryl optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷. In a preferred embodiment, R⁵ is phenyl or pyridin-2-yl optionally substituted with one to two groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷.

In yet another embodiment, the invention relates to compounds of formula Id wherein Y is —NC(O)—R¹⁰ wherein R¹⁰ is selected from lower alkyl optionally substituted with one to eight groups selected from halo, hydroxyl, lower alkenyl, lower alkynyl, lower alkoxy, —NR⁸R′; or R¹⁰ is selected from cycloalkyl, cycloalkylalkyl, heterocyclyl, heterocyclylalkyl, aryl, arylalkyl, heteroaryl or heteroarylalkyl, the ring portion of each is optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷; or aryl or heteroaryl optionally substituted with one or two groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷. In a preferred embodiment, R¹⁰ is lower alkyl substituted with 1-3 groups independently selected from halo, or R¹⁰ is aryl or heteroaryl optionally substituted with lower alkyl or halo.

In still another embodiment, the invention relates to compounds for formula Id wherein X and Y form the c ring as defined above for formula I. In a preferred embodiment, the c ring is that as shown in formula Ib.

In another embodiment, the invention relates to compounds for formula Id wherein R²⁵ is hydrogen.

The invention also relates to compounds of formula Ie:

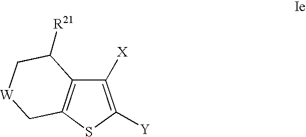

wherein X and Y are as defined above for formula I and W is selected from —O—, —S—, —C(R²⁶)(R²⁸)— and —NR³⁰—, wherein R²¹ is hydrogen or lower alkyl and R²⁶, R²⁸ and R³⁰ are independently selected from optionally substituted with one to three groups independently selected from lower alkyl, halo, oxo, nitro, —CN, lower alkenyl, lower alkynyl, trifluoromethyl, —C(O)R⁷, C(O)OR′, —C(O)NR^{7′}R⁷ and —NHC(O)R⁷; or aryl or heteroaryl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷; or R²⁶ and R²⁸ together can form a cycloalkyl or heterocycloalkyl of from between 4-6 members.

In an embodiment, the invention relates to compounds of formula Ie wherein X is —C(O)NR⁵R⁶.

In still another embodiment, the invention relates to compounds of formula Ie wherein R⁶ is hydrogen and R⁵ is aryl or heteroaryl optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷. In a preferred embodiment, R⁵ is phenyl or pyridin-2-yl optionally substituted with one to two groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷.

In yet another embodiment, the invention relates to compounds of formula Ie wherein Y is —NC(O)—R¹⁰ wherein R¹⁰ is selected from lower alkyl optionally substituted with one to eight groups selected from halo, hydroxyl, lower alkenyl, lower alkynyl, lower alkoxy, —NR⁸R′; or R¹⁰ is selected from cycloalkyl, cycloalkylalkyl, heterocyclyl, heterocyclylalkyl, aryl, arylalkyl, heteroaryl or heteroarylalkyl, the ring portion of each is optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷; or aryl or heteroaryl optionally substituted with one or two groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷. In a preferred embodiment, R¹⁰ is lower alkyl substituted with 1-3 groups independently selected from halo, or R¹⁰ is aryl or heteroaryl optionally substituted with lower alkyl or halo.

In still another embodiment, the invention relates to compounds for formula Ie wherein X and Y form the c ring as defined above for formula I. In a preferred embodiment, the c ring is that as shown in formula Ib.

In still another embodiment, the invention relates to compounds for formula Ie wherein R²⁶ and R²⁸ are selected from hydrogen and 1,3-dioxolan-2-yl.

In still another embodiment, the invention relates to compounds for formula Ie wherein R³⁰ is hydrogen or —C(O)OR′, wherein R′ is as defined above for formula I.

The invention also relates to compounds of formula If:

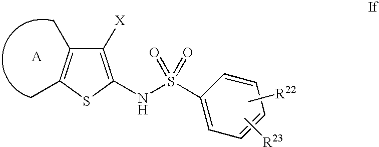

wherein the A ring and X are as defined above for formula I and R²² and R²³ are independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷; or aryl or heteroaryl optionally substituted with one or two groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷.

In an embodiment, the invention relates to compounds of formula If wherein X is —C(O)NR⁵R⁶.

In still another embodiment, the invention relates to compounds of formula If wherein R⁶ is hydrogen and R⁵ is aryl or heteroaryl optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷. In a preferred embodiment, R⁵ is phenyl or pyridin-2-yl optionally substituted with one to two groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷.

In yet another embodiment, the invention relates to compounds of formula If wherein the A ring is a 6-10 membered aryl, heteroaryl, cycloalkyl or heterocycloalkyl ring, each of which is optionally substituted with one to three groups independently selected from lower alkyl, halo, oxo, nitro, —CN, lower alkenyl, lower alkynyl, trifluoromethyl, —C(O)R⁷, C(O)OR′, —C(O)NR^{7′}R⁷ and —NHC(O)R⁷; or aryl or heteroaryl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷.

In another embodiment, the invention relates to compounds of formula If wherein R²² is hydrogen and R²³ is selected from hydrogen, halo and lower alkyl.

The invention also relates to compounds of formula Ig:

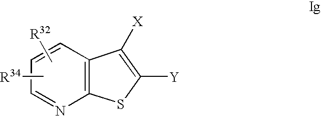

wherein X and Y are as defined above for formula I and R³² and R³⁴ are independently selected from lower alkyl, halo, nitro, —CN, lower alkenyl, lower alkynyl, trifluoromethyl, —C(O)R⁷, C(O)OR′, —C(O)NR^{7′}R⁷ and —NHC(O)R⁷; or aryl, heteroaryl, cycloalkyl or heterocycloalkyl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷.

In an embodiment, the invention relates to compounds of formula Ig wherein X is —NR^{5′}R⁶. In a preferred embodiment, R^{5′} and R⁶ are both hydrogen.

In yet another embodiment, the invention relates to compounds of formula Ig wherein Y is —NC(O)—R¹⁰ wherein R¹⁰ is selected from lower alkyl optionally substituted with one to eight groups selected from halo, hydroxyl, lower alkenyl, lower alkynyl, lower alkoxy, —NR⁸R′; or R¹⁰ is selected from cycloalkyl, cycloalkylalkyl, heterocyclyl, heterocyclylalkyl, aryl, arylalkyl, heteroaryl or heteroarylalkyl, the ring portion of each is optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷; or aryl or heteroaryl optionally substituted with one or two groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷. In a preferred embodiment, R¹⁰ is lower alkyl substituted with 1-3 groups independently selected from halo, or R¹⁰ is aryl or heteroaryl optionally substituted with lower alkyl or halo.

In still another embodiment, the invention relates to compounds for formula Ig wherein R³² is hydrogen and R³⁴ is selected from aryl or heteroaryl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷.

The invention also relates to compounds of formula Ih:

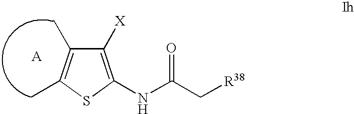

wherein the A ring and X are as described above for formula I and R³⁸ is selected from aryl or heteroaryl, each of which is optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷; or aryl or heteroaryl optionally substituted with one or two groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷.

In an embodiment, the invention relates to compounds of formula If wherein X is —C(O)NR⁵R⁶.

In yet another embodiment, the invention relates to compounds of formula If wherein R³⁸ is aryl or heteroaryl optionally substituted by one or two groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷; or aryl or heteroaryl optionally substituted with one or two groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷.

The invention also relates to compounds of formula Ii:

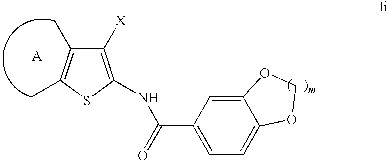

wherein the A ring and X are as defined above for formula I and m is 1 or 2.

In an embodiment, the invention relates to compounds of formula Ii wherein X is —NR^{5′}R⁶. In a preferred embodiment, R^{5′} and R⁶ are both hydrogen.

In another embodiment, the invention relates to compounds of the formula Ii wherein the A ring is a 6-membered aryl or heteroaryl group optionally substituted by one or two groups selected from lower alkyl, halo, nitro, —CN, lower alkenyl, lower alkynyl, trifluoromethyl, —C(O)R⁷, C(O)OR′, —C(O)NR^{7′}R⁷ and —NHC(O)R⁷; or aryl, heteroaryl, cycloalkyl or heterocycloalkyl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷.

The invention also relates to compounds of formula Ij:

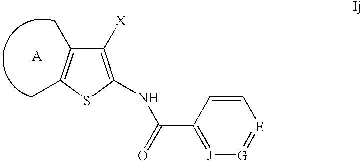

wherein the A ring and X are as defined above for formula I and one of E, G and J is N and the other two are C—R²², wherein R²² is hydrogen or as defined above for R²² in formula If.

In an embodiment, the invention relates to compounds of formula If wherein X is —C(O)NR⁵R⁶.

In yet another embodiment, one of E or G is N and the other two of E, G or J is C—R^{22′}.

The compounds of the invention include pharmaceutically acceptable salts, esters, amides, and prodrugs thereof, including but not limited to carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M. et al., “Pharmaceutical Salts,” J. Pharm. Sci., 1977; 66:1-19 which is incorporated herein by reference.)

Examples of pharmaceutically acceptable, non-toxic esters of the compounds of this invention include C₁-C₆ alkyl esters, wherein the alkyl group is a straight or branched, substituted or unsubstituted, C₅-C₇ cycloalkyl esters, as well as arylalkyl esters such as benzyl and triphenylmethyl. C₁-C₄ alkyl esters are preferred, such as methyl, ethyl, 2,2,2-trichloroethyl, and tert-butyl. Esters of the compounds of the present invention may be prepared according to conventional methods.

Examples of pharmaceutically acceptable, non-toxic amides of the compounds of this invention include amides derived from ammonia, primary C₁-C₆ alkyl amines and secondary C₁-C₆ dialkyl amines, wherein the alkyl groups are straight or branched. In the case of secondary amines, the amine may also be in the form of a 5- or 6-membered heterocycle containing one nitrogen atom. Amides derived from ammonia, C₁-C₃ alkyl primary amines and C₁-C₂ dialkyl secondary amines are preferred. Amides of the compounds of the invention may be prepared according to conventional methods.

The term “prodrug” refers to compounds that are rapidly transformed in vivo to yield the parent compound of the above formulae, for example, by hydrolysis in blood. A thorough discussion of prodrugs is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference.

These compounds can be administered individually or in combination, usually in the form of a pharmaceutical composition. Such compositions are prepared in a manner well known in the pharmaceutical art and comprise at least one active compound. Accordingly, a further aspect of the present invention includes pharmaceutical compositions comprising as one or more compounds of the invention disclosed above, associated with a pharmaceutically acceptable carrier. For administration, the compounds are ordinarily combined with one or more adjuvants appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulfuric acids, acacia, gelatin, sodium alginate, polyvinylpyrrolidine, and/or polyvinyl alcohol, and tableted or encapsulated for conventional administration. Alternatively, the compounds of this invention may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

In another aspect, the present invention provides methods for treating a subject with a tumor, comprising administering to the subject an amount effective of a compound according to formula II:

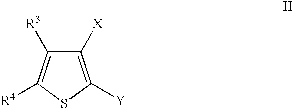

wherein
Y is —NR¹R² and X is —C(O)NR⁵R⁶ or —C(O)OR⁶, or
Y is —C(O)NR¹R⁵ and X is —NR^{5′}R⁶, or
Y is NO₂ and X is CH═CH—C(O)OR′;
R¹ and R′ are independently selected from hydrogen or lower alkyl;
R² is selected from hydrogen, —C(O)R¹⁰, —C(O)CH₂OC(O)CH₃, —SO₂R¹⁰;
R⁶ is hydrogen, lower alkyl, —SO₂R^{10′}, or
R² and R⁶, or R² and R⁸, both when Y is —NR¹R², together with respective nitrogen atoms to which they are attached are connected to form a 6-10 membered ring C, which can include a double bond and/or a fused bicyclic ring, wherein Z is —N(R⁵)— or —O—,

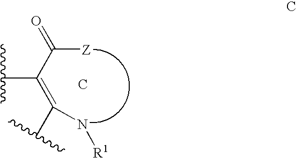

which can be optionally substituted with one or two groups selected from lower alkyl, lower alkenyl, lower alkynyl, trifluoromethyl, difluoromethyl, chlorodifluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷;
R³ and R⁴ are independently selected from hydrogen, lower alkyl, lower alkenyl, lower alkynyl, lower alkoxy, halo, —C(O)OR′, —C(O)NHR^{5″}, or
R³ is aryl optionally substituted with lower alkyl, lower alkoxy or halo, or
R³ and R⁴ together with the carbon atoms to which they are attached form a 5-14 membered aryl, heteroaryl, cycloalkyl or heterocycloalkyl ring, each of which is optionally substituted with one to three groups independently selected from lower alkyl, halo, oxo, nitro, —CN, lower alkenyl, lower alkynyl, trifluoromethyl, —C(O)R⁷, C(O)OR′, —C(O)NR^{7′}R⁷ and —NHC(O)R⁷, or
   aryl or heteroaryl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷;
R⁵, R^{5′} and R^{5″} are independently hydrogen, or
R⁵, R^{5′} and R^{5″} are independently lower alkyl optionally substituted with one to five groups selected from halo, hydroxyl, lower alkoxy, lower alkenyl and lower alkynyl, or
R⁵, R^{5′} and R^{5″} are independently selected from cycloalkyl, cycloalkylalkyl, heterocyclyl, heterocyclylalkyl, aryl, arylalkyl, heteroaryl or heteroarylalkyl, the ring portion of each is optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷, or
R⁵ and R⁶ together with the nitrogen atom to which they are attached form a 5-7 membered heterocyclic ring optionally substituted with one to three groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷;
R⁷ and R^{7′} are independently selected from hydrogen, lower alkyl, aryl, heteroaryl, arylalkyl, heteroarylalkyl, —C(O)R′ or —C(O)OR⁷;
R⁸ and R′ are independently selected from hydrogen, lower alkyl and lower alkenyl;
R¹⁰ and R^{10′} are independently selected from —NHR¹⁵—C(O)OR′, or
R¹⁰ and R^{10′} are independently lower alkyl optionally substituted with one to eight groups selected from halo, hydroxyl, lower alkenyl, lower alkynyl, lower alkoxy, —NR⁸R′, or
R¹⁰ and R^{10′} are independently selected from cycloalkyl, cycloalkylalkyl, heterocyclyl, heterocyclylalkyl, aryl, arylalkyl, heteroaryl or heteroarylalkyl, the ring portion of each is optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷, or
   aryl or heteroaryl optionally substituted with one or two groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, —CN, —SR′, —SO²R′, —C(O)R⁷, C(O)NR^{7′}R⁷ and —NHC(O)R⁷; and
R¹⁵ is lower alkyl, aryl or heteroaryl;
   and pharmaceutically acceptable derivatives thereof.

The invention also relates to methods for treating a subject with a tumor, comprising administering to the subject an effective amount of a compound according to formula I and formulas Ia-Ij as defined above, wherein the A ring, X and Y are as defined above for formula I.

The invention also relates to methods for treating a subject with a tumor, comprising administering to the subject an effective amount of a compound according to formula IIa:

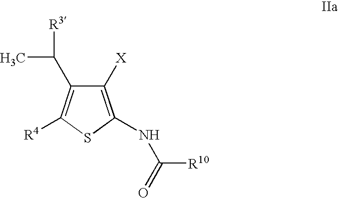

wherein X, R⁴ and R¹⁰ are as defined above for formula II and R^{3′} is hydrogen or lower alkyl.

The invention also relates to methods for treating a subject with a tumor, comprising administering to the subject an effective amount of a compound according to formula IIb:

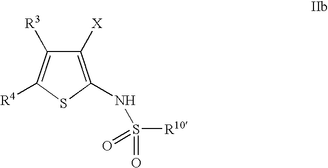

wherein X, R³, R⁴ and R^{10′} is lower alkyl.

The invention also relates to the compounds of formula I and II and to methods for treating a subject with a tumor by administering to a subject the following compounds (all compounds are named via the structure naming plug-in to either ChemDraw Ultra 8.0 and ACDLabs version 6.0, both using IUPAC rules):
2-(2,2,2-trifluoroacetamido)-4,5,6,7,8,9,10,11,12,13-decahydrocyclododeca[b]thiophene-3-carboxamide;
N-(3-carbamoyl-4,5-dimethylthiophen-2-yl)-2-(3,4-dimethoxyphenyl)quinoline-4-carboxamide;
N-(3-carbamoyl-5-methyl-4-phenylthiophen-2-yl)-8-methoxy-2-oxo-2H-chromene-3-carboxamide;
5-(2-(5-methylfuran-2-yl)quinoline-4-carboxamido)-N2,N2-diethyl-3-methylthiophene-2,4-dicarboxamide;
2-(2-(4-tert-butylphenyl)cyclopropanecarboxamido)-4-(4-fluorophenyl)-5-methylthiophene-3-carboxamide;
N-(3-carbamoyl-4-(4-ethylphenyl)-5-methylthiophen-2-yl)-2-(5-methylfuran-2-yl)quinoline-4-carboxamide;
ethyl 5-(4-bromo-1-ethyl-1H-pyrazole-5-carboxamido)-4-carbamoyl-3-methylthiophene-2-carboxylate;
N-(3-carbamoyl-5-methyl-4-(3,4-dimethylphenyl)thiophen-2-yl)-2-(5-methylfuran-2-yl)quinoline-4-carboxamide;
2-({3-[hydroxy(oxido)amino]-4-methoxybenzoyl}amino)-5-methyl-4-phenylthiophene-3-carboxamide;
methyl 5-(2-(5-methylfuran-2-yl)quinoline-4-carboxamido)-4-carbamoyl-3-methylthiophene-2-carboxylate;
N-[3-(aminocarbonyl)-4-(4-fluorophenyl)-5-methyl-2-thienyl]-2-(5-methyl-2-furyl)quinoline-4-carboxamide;
N-(3-carbamoyl-5-methyl-4-phenylthiophen-2-yl)-2-(5-methylfuran-2-yl)quinoline-4-carboxamide;
N-(3-carbamoyl-5,6-dihydro-4H-cyclopenta[b]thiophen-2-yl)-7-(difluoromethyl)-5-phenylpyrazolo[1,5-a]pyrimidine-3-carboxamide;
2-(2-ethoxybenzamido)-4,5-dimethylthiophene-3-carboxamide
methyl 4-(2-(3-fluorobenzamido)-4,5,6,7-tetrahydrobenzo[b]thiophene-3-carboxamido)benzoate;
2-(2,2,2-trifluoroacetamido)-N-(4-chlorophenyl)-4-isopropylthiophene-3-carboxamide;
2-(2,2,2-trifluoroacetamido)-4-isopropyl-N-(4-(methylthio)phenyl)thiophene-3-carboxamide;
2-(2,2,2-trifluoroacetamido)-N-(5-chloropyridin-2-yl)-4-isopropylthiophene-3-carboxamide;
2-(2,2,2-trifluoroacetamido)-N-(3-(trifluoromethyl)phenyl)-4-isopropylthiophene-3-carboxamide;
2-(2,2,2-trifluoroacetamido)-N-(4-chlorophenyl)thiophene-3-carboxamide;
2-(2,2,2-trifluoroacetamido)-N-(3-(trifluoromethyl)phenyl)thiophene-3-carboxamide;
2-(2,2,2-trifluoroacetamido)-N-(4-(methylthio)phenyl)thiophene-3-carboxamide;
2,5-bis(3,4-dichlorophenyl)-4-hydroxythiophen-3(2H)-one 1,1-dioxide;
2-(2,2,2-trifluoroacetamido)-4-isopropylthiophene-3-carboxylic acid;
N-(2-(2-(2-(2-(5-(3,4-dichlorophenylamino)-[1,2,5]oxadiazolo[3,4-b]pyrazin-6-ylamino)ethoxy)ethoxy)ethoxy)ethyl)-5-(hexahydro-2-oxo-1H-thieno[3,4-d]imidazol-6-yl)pentanamide;
tert-butyl 2-(2,2,2-trifluoroacetamido)-4-isopropylthiophene-3-carboxylate tert-butyl 2-amino-4-isopropylthiophene-3-carboxylate;
2-(trifluoromethyl)-5-isopropyl-3-(4-(methylthio)phenyl)thieno[2,3-d]pyrimidin-4(3H)-one;
2-(2,2,2-trifluoroacetamido)-N-(3-(trifluoromethyl)phenyl)-4,5,6,7,8,9-hexahydrocycloocta[b]thiophene-3-carboxamide;
2-(2,2-difluoroacetamido)-N-(4-chlorophenyl)-5,6,7,8-tetrahydro-4H-cyclohepta[b]thiophene-3-carboxamide;
N-(4-chlorophenyl)-N-[(4-chlorophenyl)sulfonyl]-2-{[(4-chlorophenyl)sulfonyl]amino}-5,6,7,8-tetrahydro-4H-cyclohepta[b]thiophene-3-carboxamide;
3-(4-tert-butylphenyl)-2-(trifluoromethyl)-3,5,6,7,8,9-hexahydro-4H-cyclohepta[4,5]thieno[2,3-d]pyrimidin-4-one;
tert-butyl 2-(2,2,2-trifluoroacetamido)-4-isopropylthiophene-3-carboxylate
2-(2,2,2-trifluoroacetamido)-4-isopropyl-N-(4-(methylthio)phenyl)thiophene-3-carboxamide;
2-(2,2,2-trifluoroacetamido)-N-(3-(trifluoromethyl)phenyl)-4,5,6,7-tetrahydro-4-methylbenzo[b]thiophene-3-carboxamide;
N-(4-chlorophenyl)-N-(phenylsulfonyl)-2-[(phenylsulfonyl)amino]-5,6,7,8-tetrahydro-4H-cyclohepta[b]thiophene-3-carboxamide;
N-(4-chlorophenyl)-2-{[(4-chlorophenyl)sulfonyl]amino}-5,6,7,8-tetrahydro-4H-cyclohepta[b]thiophene-3-carboxamide;
2-(2,2,2-trifluoroacetamido)-N-(4-tert-butylphenyl)-5,6,7,8-tetrahydro-4H-cyclohepta[b]thiophene-3-carboxamide;
tert-butyl 2-aminothiophene-3-carboxylate;
2-(2,2,2-trifluoroacetamido)thiophene-3-carboxylic acid;
2-(2,2,2-trifluoroacetamido)-N-(3-(trifluoromethyl)phenyl)-4-isopropylthiophene-3-carboxamide;
N-(4-chlorophenyl)-2-[(phenylsulfonyl)amino]-5,6,7,8-tetrahydro-4H-cyclohepta[b]thiophene-3-carboxamide;
2-(2,2,2-trifluoroacetamido)-N-(4-(trifluoromethyl)phenyl)-5,6,7,8-tetrahydro-4H-cyclohepta[b]thiophene-3-carboxamide;
1-(3-(4-chlorophenylcarbamoyl)-5,6,7,8-tetrahydro-4H-cyclohepta[b]thiophen-2-yl)-3-phenylurea;
2-(2,2,2-trifluoroacetamido)-4-isopropylthiophene-3-carboxylic acid;
2-(2,2,2-trifluoroacetamido)-N-(4-chlorophenyl)thiophene-3-carboxamide;
2-(2,2,2-trifluoroacetamido)-N-(3-(trifluoromethyl)phenyl)thiophene-3-carboxamide;
N-(butylsulfonyl)-2-[(butylsulfonyl)amino]-N-(4-chlorophenyl)-5,6,7,8-tetrahydro-4H-cyclohepta[b]thiophene-3-carboxamide;
2-(2,2,2-trifluoroacetamido)-N-(3-bromophenyl)-5,6,7,8-tetrahydro-4H-cyclohepta[b]thiophene-3-carboxamide;
1-(3-(4-chlorophenylcarbamoyl)-5,6,7,8-tetrahydro-4H-cyclohepta[b]thiophen-2-yl)-3-ethylurea;
tert-butyl 2-(2,2,2-trifluoroacetamido)thiophene-3-carboxylate;
2-(2,2,2-trifluoroacetamido)-N-(4-(methylthio)phenyl)thiophene-3-carboxamide;
2-(2,2,3,3,4,4,4-heptafluorobutanamido)-N-(4-chlorophenyl)-5,6,7,8-tetrahydro-4H-cyclohepta[b]thiophene-3-carboxamide;
2-[(butylsulfonyl)amino]-N-(4-chlorophenyl)-5,6,7,8-tetrahydro-4H-cyclohepta[b]thiophene-3-carboxamide;
3-(4-ethylphenyl)-2-(trifluoromethyl)-3,5,6,7,8,9-hexahydro-4H-cyclohepta[4,5]thieno[2,3-d]pyrimidin-4-one;
2-(trifluoromethyl)-3-[3-(trifluoromethyl)phenyl]-5,6,7,8-tetrahydro[1]benzothieno[2,3-d]pyrimidin-4(3H)-one;
2-(2,2,2-trifluoroacetamido)-N-(5-chloropyridin-2-yl)-4,5,6,7-tetrahydro-4-methylbenzo[b]thiophene-3-carboxamide;
2-(2,2,2-trifluoroacetamido)-N-(4-chlorophenyl)-5,7-dihydro-4H-thieno[2,3-c]pyran-3-carboxamide;
N-(4-chlorophenyl)-2-(glycoloylamino)-5,6,7,8-tetrahydro-4H-cyclohepta[b]thiophene-3-carboxamide;
N-(4-chlorophenyl)-N-[(4-fluorophenyl)sulfonyl]-2-{[(4-fluorophenyl)sulfonyl]amino}-5,6,7,8-tetrahydro-4H-cyclohepta[b]thiophene-3-carboxamide;
2-(2,2,2-trifluoroacetamido)-N-(4-ethylphenyl)-5,6,7,8-tetrahydro-4H-cyclohepta[b]thiophene-3-carboxamide;
2-(2,2,2-trifluoroacetamido)-N-(3-(trifluoromethyl)phenyl)-4,5,6,7-tetrahydrobenzo[b]thiophene-3-carboxamide;
2-(2,2,2-trifluoroacetamido)-N-(4-chlorophenyl)-4-isopropylthiophene-3-carboxamide;
2-(2,2,2-trifluoroacetamido)-5,7-dihydro-N-(4-(methylthio)phenyl)-4H-thieno[2,3-c]pyran-3-carboxamide;
2-[chloro(difluoro)methyl]-3-(4-chlorophenyl)-3,5,6,7,8,9-hexahydro-4H-cyclohepta[4,5]thieno[2,3-d]pyrimidin-4-one;
N-(4-chlorophenyl)-2-{[(4-fluorophenyl)sulfonyl]amino}-5,6,7,8-tetrahydro-4H-cyclohepta[b]thiophene-3-carboxamide;
3-(4-butylphenyl)-2-(trifluoromethyl)-3,5,6,7,8,9-hexahydro-4H-cyclohepta[4,5]thieno[2,3-d]pyrimidin-4-one;
2-(2,2,2-trifluoroacetamido)-6-acetyl-N-(3-(trifluoromethyl)phenyl)-4,5,6,7-tetrahydrothieno[2,3-c]pyridine-3-carboxamide;
2-(2,2,2-trifluoroacetamido)-N-(5-chloropyridin-2-yl)-4-isopropylthiophene-3-carboxamide;
2-(2,2,2-trifluoroacetamido)-N-(3-(trifluoromethyl)phenyl)-5,7-dihydro-4H-thieno[2,3-c]pyran-3-carboxamide;
2-(2-chloro-2,2-difluoroacetamido)-N-(4-chlorophenyl)-5,6,7,8-tetrahydro-4H-cyclohepta[b]thiophene-3-carboxamide;
2-(trifluoromethyl)-3-[4-(trifluoromethyl)phenyl]-3,5,6,7,8,9-hexahydro-4H-cyclohepta[4,5]thieno[2,3-d]pyrimidin-4-one;
2-(2,2,2-trifluoroacetamido)-N-(4-butylphenyl)-5,6,7,8-tetrahydro-4H-cyclohepta[b]thiophene-3-carboxamide;
tert-butyl 2-amino-4-isopropylthiophene-3-carboxylate;
2-(2,2,2-trifluoroacetamido)-N-(3-chlorophenyl)-5,6,7,8-tetrahydro-4H-cyclohepta[b]thiophene-3-carboxamide;
3-(4-methylpyridin-2-yl)-2-(trifluoromethyl)-3,5,6,7,8,9-hexahydro-4H-cyclohepta[4,5]thieno[2,3-d]pyrimidin-4-one;
tert-butyl 2-(2,2,2-trifluoroacetamido)-4,5,6,7,8,9-hexahydrocycloocta[b]thiophene-3-carboxylate;
N-[3-(4-chlorobenzoyl)-5,6,7,8-tetrahydro-4H-cyclohepta[b]thien-2-yl]-2,2,2-trifluoroacetamide;
3-[4-(methylthio)phenyl]-2-(trifluoromethyl)-5,6,7,8-tetrahydro[1]benzothieno[2,3-d]pyrimidin-4(3H)-one;
3-phenyl-2-(trifluoromethyl)-3,5,6,7,8,9-hexahydro-4H-cyclohepta[4,5]thieno[2,3-d]pyrimidin-4-one;
2,2,2-trifluoro-N-[3-(morpholin-4-ylcarbonyl)-5,6,7,8-tetrahydro-4H-cyclohepta[b]thien-2-yl]acetamide;
2-(2,2,2-trifluoroacetamido)-5,7-dihydro-4H-thieno[2,3-c]pyran-3-carboxylic acid;
(2-amino-5,6,7,8-tetrahydro-4H-cyclohepta[b]thiophen-3-yl)(3-(trifluoromethyl)phenyl)methanone;
3-(4-chlorophenyl)-2-(trifluoromethyl)-3,5,6,7,8,9-hexahydro-4H-cyclohepta[4,5]thieno[2,3-d]pyrimidin-4-one;
3-pyridin-2-yl-2-(trifluoromethyl)-3,5,6,7,8,9-hexahydro-4H-cyclohepta[4,5]thieno[2,3-d]pyrimidin-4-one;
tert-butyl 2-[(trifluoroacetyl)amino]-4,5,7,7a-tetrahydro-3aH-spiro[1-benzothiophene-6,2′-[1,3]dioxolane]-3-carboxylate;
N-[3-(3-chlorobenzoyl)-5,6,7,8-tetrahydro-4H-cyclohepta[b]thien-2-yl]-2,2,2-trifluoroacetamide;
3-(3,4-dichlorophenyl)-2-(trifluoromethyl)-3,5,6,7,8,9-hexahydro-4H-cyclohepta[4,5]thieno[2,3-d]pyrimidin-4-one;
3-pyridin-3-yl-2-(trifluoromethyl)-3,5,6,7,8,9-hexahydro-4H-cyclohepta[4,5]thieno[2,3-d]pyrimidin-4-one;
2-(2,2,2-trifluoroacetamido)-4,5,6,7,8,9-hexahydrocycloocta[b]thiophene-3-carboxylic acid;
2,2,2-trifluoro-N-{3-[3-(trifluoromethyl)benzoyl]-5,6,7,8-tetrahydro-4H-cyclohepta[b]thien-2-yl}acetamide;
3-(3,5-dichlorophenyl)-2-(trifluoromethyl)-3,5,6,7,8,9-hexahydro-4H-cyclohepta[4,5]thieno[2,3-d]pyrimidin-4-one;
3-(3-chlorophenyl)-2-(trifluoromethyl)-3,5,6,8-tetrahydro-4H-pyrano[4′,3′:4,5]thieno[2,3-d]pyrimidin-4-one;
(2-amino-5,6,7,8-tetrahydro-4H-cyclohepta[b]thiophen-3-yl)(4-chlorophenyl)methanone;
2-(2,2,2-trifluoroacetamido)-N-(3-chlorophenyl)-5,6,7,8-tetrahydro-4H-cyclohepta[b]thiophene-3-carboxamide;
3-(5-chloropyridin-2-yl)-2-(trifluoromethyl)-3,5,6,7,8,9-hexahydro-4H-cyclohepta[4,5]thieno[2,3-d]pyrimidin-4-one;
3-pyridin-3-yl-2-(trifluoromethyl)-3,5,6,8-tetrahydro-4H-pyrano[4′,3′:4,5]thieno[2,3-d]pyrimidin-4-one;
2-(2,2,2-trifluoroacetamido)-4,5,6,7-tetrahydro-4-methylbenzo[b]thiophene-3-carboxylic acid;
3-(5-chloropyridin-2-yl)-2-(trifluoromethyl)-3,5,6,7,8,9-hexahydro-4H-cyclohepta[4,5]thieno[2,3-d]pyrimidin-4-one;
3-(5-methylpyridin-2-yl)-2-(trifluoromethyl)-3,5,6,7,8,9-hexahydro-4H-cyclohepta[4,5]thieno[2,3-d]pyrimidin-4-one;
3-pyridin-4-yl-2-(trifluoromethyl)-3,5,6,8-tetrahydro-4H-pyrano[4′,3′:4,5]thieno[2,3-d]pyrimidin-4-one;
(2-amino-5,6,7,8-tetrahydro-4H-cyclohepta[b]thiophen-3-yl)(3-chlorophenyl)methanone;
3-(3,4-dimethylphenyl)-2-(trifluoromethyl)-3,5,6,7,8,9-hexahydro-4H-cyclohepta[4,5]thieno[2,3-d]pyrimidin-4-one;
3-pyridin-4-yl-2-(trifluoromethyl)-3,5,6,7,8,9-hexahydro-4H-cyclohepta[4,5]thieno[2,3-d]pyrimidin-4-one;
tert-butyl 2-(2,2,2-trifluoroacetamido)-4,5,6,7-tetrahydro-4-methylbenzo[b]thiophene-3-carboxylate;
3-(4-chlorophenyl)-2-(trifluoromethyl)-3,5,6,8-tetrahydro-4H-spiro[1-benzothieno[2,3-d]pyrimidine-7,2′-[1,3]dioxolan]-4-one;
(Z)-2-(2,2,2-trifluoroacetamido)-N-(3-chlorophenyl)-7,8-dihydro-6H-cyclohepta[b]thiophene-3-carboxamide;
3-amino-N-(4-bromo-2-methylphenyl)-6-(3,4-dimethoxyphenyl)thieno[2,3-b]pyridine-2-carboxamide;
3-amino-N-(2-(butylthio)phenyl)-6-(thiophen-2-yl)thieno[2,3-b]pyridine-2-carboxamide;
N-(4-chlorophenyl)-2-[(trifluoroacetyl)amino]-4,7-dihydro-5H-spiro[1-benzothiophene-6,2′-[1,3]dioxolane]-3-carboxamide;
2-(2,2,2-trifluoroacetamido)-N-(2,2,2-trifluoroethyl)-5,6,7,8-tetrahydro-4H-cyclohepta[b]thiophene-3-carboxamide;
3-amino-N-(2,3-dihydrobenzo[b][1,4]dioxin-7-yl)-6-(3-methoxyphenyl)thieno[2,3-b]pyridine-2-carboxamide;
3-(4-chlorophenyl)-2-(trifluoromethyl)-5,6,7,8,9, I O-hexahydrocycloocta[4,5]thieno[2,3-d]pyrimidin-4(3H)-one;
3-(4-chlorophenyl)-2-(trifluoromethyl)-5,6,7,8-tetrahydro[1]benzothieno[2,3-d]pyrimidin-4(3H)-one;
2-(2-(3-(trifluoromethyl)-5-methyl-1H-pyrazol-1-yl)acetamido)-6-tert-butyl-4,5,6,7-tetrahydrobenzo[b]thiophene-3-carboxamide;
3-amino-N-(3-ethoxyphenyl)-6-(3,4-dimethoxyphenyl)thieno[2,3-b]pyridine-2-carboxamide;
3-amino-N-(benzo[d][1,3]dioxol-6-yl)-6-(3-methoxyphenyl)thieno[2,3-b]pyridine-2-carboxamide;
2-(2,2,2-trifluoroacetamido)-N-(4-chlorophenyl)-4,5,6,7,8,9-hexahydrocycloocta[b]thiophene-3-carboxamide;
2-(2,2,2-trifluoroacetamido)-N-(4-chlorophenyl)-4,5,6,7-tetrahydrobenzo[b]thiophene-3-carboxamide;
2-(2-(4-bromo-3-(trifluoromethyl)-5-methyl-1H-pyrazol-1-yl)acetamido)-5,6,7,8-tetrahydro-4H-cyclohepta[b]thiophene-3-carboxamide;
3-amino-N-(3,4,5-trimethoxyphenyl)-6-(thiophen-2-yl)thieno[2,3-b]pyridine-2-carboxamide;
3-amino-6-(3-methoxyphenyl)-N-(3-(methylthio)phenyl)thieno[2,3-b]pyridine-2-carboxamide;
3-(4-chlorophenyl)-5-methyl-2-(trifluoromethyl)-5,6,7,8-tetrahydro[1]benzothieno[2,3-d]pyrimidin-4(3H)-one;
2-(2,2,2-trifluoroacetamido)-N-(4-chlorophenyl)-5,6,7,8-tetrahydro-4H-cyclohepta[b]thiophene-3-carboxamide;
3-amino-6-(3,4-dimethoxyphenyl)-N-(3,4-dimethylphenyl)thieno[2,3-b]pyridine-2-carboxamide;
3-amino-N-(benzo[d][1,3]dioxol-6-yl)-6-(3,4-dimethoxyphenyl)thieno[2,3-b]pyridine-2-carboxamide;
3-amino-6-(3-methoxyphenyl)-N-(3,4-dimethylphenyl)thieno[2,3-b]pyridine-2-carboxamide;
2-(2,2,2-trifluoroacetamido)-N-(4-chlorophenyl)-4,5,6,7-tetrahydro-4-methylbenzo[b]thiophene-3-carboxamide;
3-amino-N-(4-bromo-3-methylphenyl)-6-(3,4-dimethoxyphenyl)thieno[2,3-b]pyridine-2-carboxamide;
3-amino-6-(4-fluorophenyl)-N-(3,4,5-trimethoxyphenyl)thieno[2,3-b]pyridine-2-carboxamide;
3-amino-N-(2-(ethylthio)phenyl)-6-(thiophen-2-yl)thieno[2,3-b]pyridine-2-carboxamide;
2-(2-methoxybenzamido)-4,5,6,7,8,9-hexahydrocycloocta[b]thiophene-3-carboxamide;
3-(3-chlorophenyl)-2-(trifluoromethyl)-3,5,6,8-tetrahydro-4H-pyrano[4′,3′:4,5]thieno[2,3-d]pyrimidin-4-one;
3-amino-N-(4-bromophenyl)-6-(3,4-dimethoxyphenyl)thieno[2,3-b]pyridine-2-carboxamide;
3-amino-N-(4-fluorophenyl)-6-(3,4-dimethoxyphenyl)thieno[2,3-b]pyridine-2-carboxamide;
3-amino-N-(2-(propylthio)phenyl)-6-(thiophen-2-yl)thieno[2,3-b]pyridine-2-carboxamide;
2-(2,2,2-trifluoroacetamido)-N-(3-chlorophenyl)-5,7-dihydro-4H-thieno[2,3-c]pyran-3-carboxamide;
3-amino-N-(3-chloro-4-methylphenyl)-6-(3,4-dimethoxyphenyl)thieno[2,3-b]pyridine-2-carboxamide;
3-amino-N-(2-bromophenyl)-6-(thiophen-2-yl)thieno[2,3-b]pyridine-2-carboxamide
2-(2,2,2-trifluoroacetamido)-N-(3-chlorophenyl)-5,6,7,8-tetrahydro-4H-cyclohepta[b]thiophene-3-carboxamide;
3-(3-chlorophenyl)-2-(trifluoromethyl)-5,6,7,8,9,10-hexahydrocycloocta[4,5]thieno[2,3-d]pyrimidin-4(3H)-one;
3-(3-chlorophenyl)-2-(trifluoromethyl)-3,5,6,8-tetrahydro-4H-spiro[1-benzothieno[2,3-d]pyrimidine-7,2′-[1,3]dioxolan]-4-one;
N-(3,4-difluorophenyl)-4,5,6,7-tetrahydrobenzo[b]thiophene-3-carboxamide;
ethyl 2-(2,2,2-trifluoroacetamido)-4-(4-chlorophenyl)-5-methylthiophene-3-carboxylate;
5,6,7,8-tetrahydro-2-(methyl carbamoylformyl)-4H-cyclohepta[b]thiophene-3-carboxylic acid;
3-[4-(methylthio)phenyl]-2-(trifluoromethyl)-3,5,6,7,8,9-hexahydro-4H-cyclohepta[4,5]thieno[2,3-d]pyrimidin-4-one;
3-(3-methoxyphenyl)-2-(trifluoromethyl)-3,5,6,7,8,9-hexahydro-4H-cyclohepta[4,5]thieno[2,3-d]pyrimidin-4-one;
3-(3-chlorophenyl)-2-(trifluoromethyl)-3,5,6,8-tetrahydro-4H-thiopyrano[4′,3′:4,5]thieno[2,3-d]pyrimidin-4-one;
3-(3-chlorophenyl)-2-(trifluoromethyl)-5,6,7,8-tetrahydropyrido[4′,3′:4,5]thieno[2,3-d]pyrimidin-4(3H)-one;
3-[4-(methylthio)phenyl]-2-(trifluoromethyl)-5,6,7,8,9,10-hexahydrocycloocta[4,5]thieno[2,3-d]pyrimidin-4(3H)-one;
2-(2,2,2-trifluoroacetamido)-4,5,6,7-tetrahydro-6-oxobenzo[b]thiophene-3-carboxylic acid;
N-(3-fluoro-4-methylphenyl)-5-methyl-4-phenylthiophene-3-carboxamide;
tert-butyl 2-(2,2,2-trifluoroacetamido)-4,5,6,7-tetrahydrobenzo[b]thiophene-3-carboxylate;
3-[4-(methylthio)phenyl]-2-(trifluoromethyl)-5,6,7,8-tetrahydro[1]benzothieno[2,3-d]pyrimidin-4(3H)-one;
2-(2,2,2-trifluoroacetamido)-4,5,6,7-tetrahydro-N-(4-(methylthio)phenyl)benzo[b]thiophene-3-carboxamide;
ethyl 3-(3-chlorophenyl)-4-oxo-3,5,6,7,8,9-hexahydro-4H-cyclohepta[4,5]thieno[2,3-d]pyrimidine-2-carboxylate;
tert-butyl 6-acetyl-2-amino-4,5,6,7-tetrahydrothieno[2,3-c]pyridine-3-carboxylate;
3-(3-chlorophenyl)-2-(trifluoromethyl)-3,5,6,8-tetrahydro-4H-thiopyrano[4′,3′:4,5]thieno[2,3-d]pyrimidin-4-one 7-oxide;
3-(3-chlorophenyl)-5-methyl-2-(trifluoromethyl)-5,6,7,8-tetrahydro[1]benzothieno[2,3-d]pyrimidin-4(3H)-one;
3-(4-chlorobenzyl)-2-(trifluoromethyl)-3,5,6,7,8,9-hexahydro-4H-cyclohepta[4,5]thieno[2,3-d]pyrimidin-4-one;
6-tert-butyl-4,5,6,7-tetrahydro-N-(3-(methylthio)phenyl)benzo[b]thiophene-3-carboxamide;
3-(4-methylphenyl)-2-(trifluoromethyl)-5,6,7,8-tetrahydro[1]benzothieno[2,3-d]pyrimidin-4(3H)-one;
3-[4-(methylsulfonyl)phenyl]-2-(trifluoromethyl)-5,6,7,8-tetrahydro[1]benzothieno[2,3-d]pyrimidin-4(3H)-one;
ethyl 3-(3-chlorophenyl)-4-oxo-3,4,5,6,7,8-hexahydro[1]benzothieno[2,3-d]pyrimidine-2-carboxylate;
tert-butyl 2-amino-5,7-dihydro-4H-thieno[2,3-c]thiopyran-3-carboxylate;
3-tert-butyl 6-propyl 2-[(trifluoroacetyl)amino]-4,7-dihydrothieno[2,3-c]pyridine-3,6(5H)-dicarboxylate;
3-(3-chlorophenyl)-2-(trifluoromethyl)-3,5,6,8-tetrahydro-4H-thiopyrano[4′,3′:4,5]thieno[2,3-d]pyrimidin-4-one 7,7-dioxide;
5-methyl-3-[4-(methylthio)phenyl]-2-(trifluoromethyl)-5,6,7,8-tetrahydro[1]benzothieno[2,3-d]pyrimidin-4(3H)-one;
3-(4-chlorophenyl)-2-(trifluoromethyl)-3,5,6,8-tetrahydro[1]benzothieno[2,3-d]pyrimidine-4,7-dione;
dimethyl 2-(2,2,2-trifluoroacetamido)-5,6-dihydro-4H-cyclopenta[b]thiophene-3,4-dicarboxylate;
3-(3-chlorophenyl)-2-(trifluoromethyl)-5,6,7,8-tetrahydro[1]benzothieno[2,3-d]pyrimidin-4(3H)-one;
3-[4-(methylsulfinyl)phenyl]-2-(trifluoromethyl)-5,6,7,8-tetrahydro[1]benzothieno[2,3-d]pyrimidin-4(3H)-one;
2-(trifluoromethyl)-3-[3-(trifluoromethyl)phenyl]-3,5,6,7,8,9-hexahydro-4H-cyclohepta[4,5]thieno[2,3-d]pyrimidin-4-one;
3-(3-chlorophenyl)-3,5,6,7,8,9-hexahydro-4H-cyclohepta[4,5]thieno[2,3-d]pyrimidin-4-one;
tert-butyl 2-(2,2,2-trifluoroacetamido)-6-acetyl-4,5,6,7-tetrahydrothieno[2,3-c]pyridine-3-carboxylate;
ethyl 4-oxo-3-(2,2,2-trifluoroethyl)-3,5,6,7,8,9-hexahydro-4H-cyclohepta[4,5]thieno[2,3-d]pyrimidine-2-carboxylate;
2-(2,2,2-trifluoroacetamido)-N-(2-chlorophenyl)-5,6,7,8-tetrahydro-4H-cyclohepta[b]thiophene-3-carboxamide;
3-(5-chloropyridin-2-yl)-2-(trifluoromethyl)-5,6,7,8,9,10-hexahydrocycloocta[4,5]thieno[2,3-a]pyrimidin-4(3H)-one;
2-ethyl 4-methyl 5-(2,2,2-trifluoroacetamido)-3-methylthiophene-2,4-dicarboxylate;
3-(5-chloropyridin-2-yl)-2-(trifluoromethyl)-5,6,7,8-tetrahydro[1]benzothieno[2,3-d]pyrimidin-4(3H)-one;
3-[3-(methylthio)phenyl]-2-(trifluoromethyl)-3,5,6,7,8,9-hexahydro-4H-cyclohepta[4,5]thieno[2,3-d]-pyrimidin-4-one;
tert-butyl 2-(ethyl carbamoylformyl)-5,6,7,8-tetrahydro-4H-cyclohepta[b]thiophene-3-carboxylate;
3-(3-chlorophenyl)-5,6,7,8-tetrahydro[1]benzothieno[2,3-d]pyrimidin-4(3H)-one;
6-(propoxycarbonyl)-2-[(trifluoroacetyl)amino]-4,5,6,7-tetrahydrothieno[2,3-c]pyridine-3-carboxylic acid;
3-(3-bromophenyl)-2-(trifluoromethyl)-3,5,6,7,8,9-hexahydro-4H-cyclohepta[4,5]thieno[2,3-d]pyrimidin-4-one;
2-(trifluoromethyl)-3-[6-(trifluoromethyl)pyridin-3-yl]-3,5,6,7,8,9-hexahydro-4H-cyclohepta[4,5]thieno[2,3-d]pyrimidin-4-one;
3-(5-chloropyridin-2-yl)-5-methyl-2-(trifluoromethyl)-5,6,7,8-tetrahydro[1]benzothieno[2,3-d]pyrimidin-4(3H)-one;
ethyl 2-(2,2,2-trifluoroacetamido)-4,5,6,7,8,9-hexahydrocycloocta[b]thiophene-3-carboxylate;
3-(5-methylpyridin-2-yl)-2-(trifluoromethyl)-5,6,7,8-tetrahydro[1]benzothieno[2,3-d]pyrimidin-4(3H)-one;
3-[3-(methylsulfonyl)phenyl]-2-(trifluoromethyl)-3,5,6,7,8,9-hexahydro-4H-cyclohepta[4,5]thieno[2,3-d]pyrimidin-4-one;
2-(ethyl carbamoylformyl)-5,6,7,8-tetrahydro-4H-cyclohepta[b]thiophene-3-carboxylic acid;
tert-butyl 2-(2,2,2-trifluoroacetamido)-5,7-dihydro-4H-thieno[2,3-c]thiopyran-3-carboxylate;
2-(2,2,2-trifluoroacetamido)-6-acetyl-4,5,6,7-tetrahydrothieno[2,3-c]pyridine-3-carboxylic acid;
3-(4-fluorophenyl)-2-(trifluoromethyl)-3,5,6,7,8,9-hexahydro-4H-cyclohepta[4,5]thieno[2,3-d]pyrimidin-4-one;
N2,N3-dibenzyl-4-bromothiophene-2,3-dicarboxamide;
ethyl 2-(2,2,2-trifluoroacetamido)-5,6,7,8-tetrahydro-4H-cyclohepta[b]thiophene-3-carboxylate;
3-(3,4-dimethylphenyl)-2-(trifluoromethyl)-5,6,7,8-tetrahydro[1]benzothieno[2,3-d]pyrimidin-4(3H)-one;
3-[3-(methylsulfinyl)phenyl]-2-(trifluoromethyl)-3,5,6,7,8,9-hexahydro-4H-cyclohepta[4,5]thieno[2,3-d]pyrimidin-4-one;
3-(3-chlorophenyl)-2-(trifluoromethyl)-3,5,6,7,8,9-hexahydro-4H-cyclohepta[4,5]thieno[2,3-d]pyrimidin-4-one;
2-(2,2,2-trifluoroacetamido)-5,7-dihydro-4H-thieno[2,3-c]thiopyran-3-carboxylic acid;
propyl 3-(3-chlorophenyl)-4-oxo-2-(trifluoromethyl)-3,5,6,8-tetrahydropyrido[4′,3′:4,5]thieno[2,3-d]pyrimidine-7(4H)-carboxylate;
2-[(trifluoroacetyl)amino]-4,7-dihydro-5H-spiro[1-benzothiophene-6,2′-[1,3]dioxolane]-3-carboxylic acid;
N,4,5-triphenylthiophene-3-carboxamide;
isopropyl 5-(4-chlorophenylcarbamoyl)-2-(2,2,2-trifluoroacetamido)-4-methylthiophene-3-carboxylate;
3-(3,4-dichlorophenyl)-2-(trifluoromethyl)-5,6,7,8-tetrahydro[1]benzothieno[2,3-d]pyrimidin-4(3H)-one;
tert-butyl 2-(ethyl carbamoylformyl)-4,5,6,7-tetrahydrobenzo[b]thiophene-3-carboxylate
3-(2,2,2-trifluoroethyl)-2-(trifluoromethyl)-3,5,6,7,8,9-hexahydro-4H-cyclohepta[4,5]thieno[2,3-d]pyrimidin-4-one;
3-tert-butyl 6-propyl 2-amino-4,7-dihydrothieno[2,3-c]pyridine-3,6(5H)-dicarboxylate
7-acetyl-3-(3-chlorophenyl)-2-(trifluoromethyl)-5,6,7,8-tetrahydropyrido[4′,3′:4,5]thieno[2,3-d]pyrimidin-4(3H)-one;
propyl 3-(4-chlorophenyl)-4-oxo-2-(trifluoromethyl)-3,5,6,8-tetrahydropyrido[4′,3′:4,5]thieno[2,3-d]pyrimidine-7(4H)-carboxylate;
2-(2,2,2-trifluoroacetamido)-5,6,7,8-tetrahydro-N-(4-(methylthio)phenyl)-4H-cyclohepta[b]thiophene-3-carboxamide;
3-(4-chlorophenyl)-2-(trifluoromethyl)quinazolin-4(3H)-one;
(Z)-tert-butyl 2-(2,2,2-trifluoroacetamido)-7,8-dihydro-6H-cyclohepta[b]thiophene-3-carboxylate;
ethyl(3-(4-chlorophenylcarbamoyl)-5,6,7,8-tetrahydro-4H-cyclohepta[b]thiophen-2-ylcarbamoyl)formate;
N-(4-chlorophenyl)-2-cyano-2-cycloheptylideneacetamide;
2-(2,2,2-trifluoroacetamido)-N-(4-chlorophenyl)benzamide;
tert-butyl 2-[(methylsulfonyl)amino]-5,6,7,8-tetrahydro-4H-cyclohepta[b]thiophene-3-carboxylate;
3-(4-chlorophenyl)-2-(trifluoromethyl)-5,6,7,8-tetrahydropyrido[4′,3′:4,5]thieno[2,3-d]pyrimidin-4(3H)-one;
2-(2,2,2-trifluoroacetamido)-N-(3-chlorophenyl)-4,5,6,7-tetrahydrobenzo[b]thiophene-3-carboxamide;
2-amino-N-(4-chlorophenyl)-5,6,7,8-tetrahydro-4H-cyclohepta[b]thiophene-3-carboxamide;
2-[(3-{[(4-chlorophenyl)amino]carbonyl}-5,6,7,8-tetrahydro-4H-cyclohepta[b]thien-2-yl)amino]-2-oxoethyl acetate;
(Z)-2-(2,2,2-trifluoroacetamido)-N-(4-chlorophenyl)-7,8-dihydro-6H-cyclohepta[b]thiophene-3-carboxamide;
propyl 3-{[(4-chlorophenyl)amino]carbonyl}-2-[(trifluoroacetyl)amino]-4,7-dihydrothieno[2,3-c]pyridine-6(5H)-carboxylate;
2-(2,2,2-trifluoroacetamido)-4,5,6,7-tetrahydro-N-(5-methylpyridin-2-yl)benzo[b]thiophene-3-carboxamide;
2-(2,2,2-trifluoroacetamido)-N-(4-chlorophenyl)-5,6,7,8-tetrahydro-4H-cyclohepta[b]thiophene-3-carboxamide;
2-acetamido-N-(4-chlorophenyl)-5,6,7,8-tetrahydro-4H-cyclohepta[b]thiophene-3-carboxamide;
2-(2,2,2-trifluoroacetamido)-N-(3-chlorophenyl)-5,7-dihydro-4H-thieno[2,3-c]thiopyran-3-carboxamide;
2-(2,2,2-trifluoroacetamido)-N-(4-chlorophenyl)-4,5,6,7-tetrahydrothieno[2,3-c]pyridine-3-carboxamide;
(3-(4-chlorophenylcarbamoyl)-5,6,7,8-tetrahydro-4H-cyclohepta[b]thiophen-2-ylcarbamoyl)formic acid;
2-(2,2,2-trifluoroacetamido)-N-(4-chlorophenyl)-4,5,6,7-tetrahydro-6-oxobenzo[b]thiophene-3-carboxamide;
2-(2,2,2-trifluoroacetamido)-N-(5-chloropyridin-2-yl)-4,5,6,7-tetrahydrobenzo[b]thiophene-3-carboxamide;
N-(3-chlorophenyl)-2-[(methylsulfonyl)amino]-5,6,7,8-tetrahydro-4H-cyclohepta[b]thiophene-3-carboxamide;
7-acetyl-3-(4-chlorophenyl)-2-(trifluoromethyl)-5,6,7,8-tetrahydropyrido[4′,3′:4,5]thieno[2,3-d]pyrimidin-4(3H)-one;
N-(4-chlorophenyl)-2-[(N,N-dimethylglycyl)amino]-5,6,7,8-tetrahydro-4H-cyclohepta[b]thiophene-3-carboxamide;
2-(trifluoromethyl)-5,6,7,8,9,10-hexahydro-4H-cycloocta[4,5]thieno[2,3-d][1,3]oxazin-4-one;
2-(2,2,3,3,3-pentafluoropropanamido)-N-(4-chlorophenyl)-5,6,7,8-tetrahydro-4H-cyclohepta[b]thiophene-3-carboxamide;
propyl 3-{([(3-chlorophenyl)amino]carbonyl}-2-[(trifluoroacetyl)amino]-4,7-dihydrothieno[2,3-c]pyridine-6(5H)-carboxylate;
2-(2-methoxyacetamido)-N-(4-chlorophenyl)-5,6,7,8-tetrahydro-4H-cyclohepta[b]thiophene-3-carboxamide;
2-(2,2,2-trifluoroacetamido)-6-acetyl-N-(4-chlorophenyl)-4,5,6,7-tetrahydrothieno[2,3-c]pyridine-3-carboxamide;
tert-butyl 2-(2,2,2-trifluoroacetamido)benzoate;
2-(2,2,2-trifluoroacetamido)-6-acetyl-N-(3-chlorophenyl)-4,5,6,7-tetrahydrothieno[2,3-c]pyridine-3-carboxamide;
N-(4-chlorophenyl)-2-(pyruvoylamino)-5,6,7,8-tetrahydro-4H-cyclohepta[b]thiophene-3-carboxamide;
2-(2,2,2-trifluoroacetamido)-N-(3-(trifluoromethyl)phenyl)-5,6,7,8-tetrahydro-4H-cyclohepta[b]thiophene-3-carboxamide;
2-(2,2,2-trifluoroacetamido)benzoic acid;
2-(2,2,2-trifluoroacetamido)-N-(3-chlorophenyl)-5,6,7,8-tetrahydro-4H-cyclohepta[b]thiophene-3-carboxamide;
N-(3-(m-tolylcarbamoyl)-6-tert-butyl-4,5,6,7-tetrahydrobenzo[b]thiophen-2-yl)isonicotinamide;
N-(3-carbamoyl-5-methyl-4-p-tolylthiophen-2-yl)-2-(5-methylfuran-2-yl)quinoline-4-carboxamide;
N-(3-carbamoyl-4-ethyl-5-methylthiophen-2-yl)-7-(trifluoromethyl)-5-phenylpyrazolo[1,5-a]pyrimidine-3-carboxamide;
N-(3-carbamoyl-4-(4-isopropylphenyl)-5-methylthiophen-2-yl)-2-(5-methylfuran-2-yl)quinoline-4-carboxamide;
ethyl 3-(4-(methoxycarbonyl)phenylcarbamoyl)-2-(2-methylbenzamido)-4,5-dihydrothieno[2,3-c]pyridine-6(7H)-carboxylate;
3-(5-chloropyridin-2-yl)-2-(trifluoromethyl)-3,5,6,7,8,9-hexahydro-4H-cyclohepta[4,5]thieno[2,3-d]pyrimidin-4-one;
N-(3-(2-chlorophenylcarbamoyl)-6-tert-butyl-4,5,6,7-tetrahydrobenzo[b]thiophen-2-yl)isonicotinamide;
N-(3-(2-methoxyphenylcarbamoyl)-6-tert-butyl-4,5,6,7-tetrahydrobenzo[b]thiophen-2-yl)nicotinamide;
N,N′-bis[3-(aminocarbonyl)-4,5,6,7-tetrahydro-1-benzothien-2-yl]hexanediamide
3-[4-(methylthio)phenyl]-2-(trifluoromethyl)-5,6,7,8-tetrahydro[1]benzothieno[2,3-d]pyrimidin-4(3H)-one;
ethyl 3-(2,4-dimethoxyphenylcarbamoyl)-2-(benzamido)-4,5-dihydrothieno[2,3-c]pyridine-6(7H)-carboxylate;
ethyl(2E)-3-{5-bromo-2-[hydroxy(oxido)amino]-3-thienyl}acrylate
N-(6-tert-pentyl-3-carbamoyl-4,5,6,7-tetrahydrobenzo[b]thiophen-2-yl)isonicotinamide;
2-(2,2,2-trifluoroacetamido)-N-(5-chloro-2-methoxyphenyl)-5,6,7,8-tetrahydro-4H-cyclohepta[b]thiophene-3-carboxamide;
3-(4-chlorophenyl)-2-(trifluoromethyl)-3,5,6,7,8,9-hexahydro-4H-cyclohepta[4,5]thieno[2,3-d]pyrimidin-4-one;
ethyl 3-(4-methoxyphenylcarbamoyl)-2-(3-fluorobenzamido)-4,5-dihydrothieno[2,3-c]pyridine-6(7H)-carboxylate;
2-[(4-tert-butylbenzoyl)amino]-N-(1,1-dioxidotetrahydro-3-thienyl)-4,5,6,7-tetrahydro-1-benzothiophene-3-carboxamide;
ethyl 3-(2,4-dimethoxyphenylcarbamoyl)-2-(4-chlorobenzamido)-4,5-dihydrothieno[2,3-c]pyridine-6(7H)-carboxylate;
3-(3,4-dichlorophenyl)-2-(trifluoromethyl)-3,5,6,7,8,9-hexahydro-4H-cyclohepta[4,5]thieno[2,3-d]pyrimidin-4-one;
ethyl 3-(4-(methoxycarbonyl)phenylcarbamoyl)-2-(2-phenylacetamido)-4,5-dihydrothieno[2,3-c]pyridine-6(7H)-carboxylate;
ethyl 3-(2,4-dimethoxyphenylcarbamoyl)-4,5-dihydro-2-(nicotinamido)thieno[2,3-c]pyridine-6(7H)-carboxylate;
2-(2-(3-(trifluoromethyl)-4,5,6,7-tetrahydroindazol-1-yl)acetamido)-4,5,6,7-tetrahydrobenzo[b]thiophene-3-carboxamide;
N-(3-carbamoyl-4,5,6,7-tetrahydrobenzo[b]thiophen-2-yl)-7-(difluoromethyl)-5-phenylpyrazolo[1,5-a]pyrimidine-3-carboxamide;
2-(2-(3-(trifluoromethyl)-4,5,6,7-tetrahydroindazol-1-yl)acetamido)-4,5,6,7-tetrahydro-6-methylbenzo[b]thiophene-3-carboxamide;
ethyl 3-(5-chloro-2-methoxyphenylcarbamoyl)-4,5-dihydro-2-(nicotinamido)thieno[2,3-c]pyridine-6(7H)-carboxylate;
5,6,7,8-tetrahydro-2-(thiophene-2-carboxamido)-N-o-tolyl-4H-cyclohepta[b]thiophene-3-carboxamide;
2-(2,2,2-trifluoroacetamido)-N-(3-chlorophenyl)-5,6,7,8-tetrahydro-4H-cyclohepta[b]thiophene-3-carboxamide;
ethyl 3-(4-(methoxycarbonyl)phenylcarbamoyl)-2-(4-fluorobenzamido)-4,5-dihydrothieno[2,3-c]pyridine-6(7H)-carboxylate;
2-(2,2,2-trifluoroacetamido)-5,6,7,8-tetrahydro-N-(naphthalen-1-yl)-4H-cyclohepta[b]thiophene-3-carboxamide;
ethyl 3-(4-methoxyphenylcarbamoyl)-2-(4-chlorobenzamido)-4,5-dihydrothieno[2,3-c]pyridine-6(7H)-carboxylate;
3-{5-[(Z)-(1,5-dioxo-6,7,8,9-tetrahydro-5H-[1]benzothieno[3,2-e][1,3]thiazolo[3,2-a]pyrimidin-2(1H)-ylidene)methyl]-2-furyl}benzoic acid;
2-(5-chloro-2-methoxybenzamido)-5,6,7,8-tetrahydro-4H-cyclohepta[b]thiophene-3-carboxamide;
2-(2,2,2-trifluoroacetamido)-N-(3-chlorophenyl)-5,6,7,8-tetrahydro-4H-cyclohepta[b]thiophene-3-carboxamide; and
N-(3-carbamoyl-4,5,6,7-tetrahydro-6-methylbenzo[b]thiophen-2-yl)-2-(2-methoxyphenyl)quinoline-4-carboxamide.

By “alkyl” and “lower alkyl” in the present invention, either alone or within other terms such as “alkylamino”, is meant straight or branched chain alkyl groups having 1-12 carbon atoms, such as, methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl. It is understood that in cases where an alkyl chain of a substituent (e.g. of an alkyl, alkoxy or alkenyl group) is within a distinct range, it will be so indicated in the second “C” as, for example, “C₁-C₆” indicates a maximum of 6 carbons. The alkyl groups herein may be substituted in one or more substitutable positions with various groups. For example, such alkyl groups may be optionally substituted with C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl, di(C₁-C₆)alkylamino(C₁-C₆)alkyl or ═O.

By “alkoxy” and “lower alkoxy” in the present invention is meant straight or branched chain alkyl groups having 1-12 carbon atoms, attached through at least one divalent oxygen atom, such as, for example, methoxy, ethoxy, propoxy, isopropoxy, n-butoxy, sec-butoxy, tert-butoxy, pentoxy, isopentoxy, neopentoxy, hexoxy, and 3-methylpentoxy. The alkoxy groups herein may be substituted in one or more substitutable positions with various groups. For example, such alkoxy groups may be optionally substituted with C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl, di(C₁-C₆)alkylamino(C₁-C₆)alkyl or ═O.

The term “alkenyl” or “lower alkyenyl” embraces linear or branched radicals having at least one carbon-carbon double bond of two to twelve atoms. More preferred alkenyl radicals are those radicals having two to about four carbon atoms. Examples of alkenyl radicals include ethenyl, 2-propenyl, allyl, butenyl and 4-methylbutenyl. The terms “alkenyl” and “lower alkenyl”, embrace radicals having “cis” and “trans” orientations, or alternatively, “E” and “Z” orientations. The alkenyl groups herein may be alkenyl groups may be optionally substituted with C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl, di(C₁-C₆)alkylamino(C₁-C₆)alkyl or ═O.

The term “alkynyl” embraces linear or branched radicals having at least one carbon-carbon triple bond of two to twelve carbon atoms. More preferred alkynyl radicals are those radicals having two to about four carbon atoms. Examples of alkynyl radicals include ethynyl, 2-propynyl, and 4-methylbutynyl. The alkynyl groups herein may be substituted in one or more substitutable positions with various groups. For example, such alkynyl groups may be optionally substituted with C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl, di(C₁-C₆)alkylamino(C₁-C₆)alkyl or ═O.

The term “halo” or “halogen” means halogens such as fluorine, chlorine, bromine or iodine atoms.

By “aryl” is meant an aromatic carbocyclic group having a single ring (e.g., phenyl), multiple rings (e.g., biphenyl), or multiple condensed rings in which at least one is aromatic, (e.g., 1,2,3,4-tetrahydronaphthyl, naphthyl), wherein such rings may be attached together in a pendent manner or may be fused. The term “aryl” embraces aromatic radicals such as phenyl, naphthyl, tetrahydronaphthyl, indane and biphenyl. More preferred aryl is phenyl. The aryl groups herein may be substituted in one or more substitutable positions with various groups. For example, such aryl groups may be optionally substituted with C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl, di(C₁-C₆)alkylamino(C₁-C₆)alkyl or ═O.

By “heteroaryl” is meant a single ring, multiple rings, or multiple condensed rings in which at least one is aromatic, wherein such rings may be attached together in a pendent manner or may be fused. The ring systems contain of from between 9-15 atoms containing at least one and up to four heteroatoms selected from nitrogen, oxygen, or sulfur. Examples include, but are not limited to, pyrrolyl, pyrrolinyl, imidazolyl, pyrazolyl, 2-pyridinyl, 3-pyridinyl, 4-pyridinyl, pyrimidinyl, pyrazinyl and pyridazinyl. The heteroaryl groups herein may be substituted in one or more substitutable positions with various groups. For example, such heteroaryl groups may be optionally substituted with C₁-C₆ alkyl, C₁-C₆ alkoxy, halogen, hydroxy, cyano, nitro, amino, mono(C₁-C₆)alkylamino, di(C₁-C₆)alkylamino; C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl, di(C₁-C₆)alkylamino(C₁-C₆)alkyl or ═O.

As used herein, the term “cycloalkyl” refers to saturated carbocyclic radicals having three to twelve carbon atoms. The cycloalkyl can be monocyclic, or a polycyclic fused or spiro system, and can optionally contain a double bond. Examples of such radicals include cyclopropyl, cyclobutyl, cyclopentyl, cyclohexyl and cycloheptyl. The cycloalkyl groups herein are unsubstituted or, as specified, substituted in one or more substitutable positions with various groups. For example, such cycloalkyl groups may be optionally substituted with C₁-C₆ alkyl, C₁-C₆ alkoxy, halogen, hydroxy, cyano, nitro, amino, oxo, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl.

By “heterocycle” or “heterocycloalkyl” is meant one or more carbocyclic ring systems which includes fused and spiro ring systems of 9-15 atoms containing at least one and up to four heteroatoms selected from nitrogen, oxygen, or sulfur. The heterocycle may optionally contain a double bond. Examples of heterocycles of the present invention include morpholinyl, thiomorpholinyl, thiomorpholinyl S-oxide, thiomorpholinyl S,S-dioxide, piperazinyl, homopiperazinyl, pyrrolidinyl, pyrrolinyl, tetrahydropyranyl, piperidinyl, tetrahydrofuranyl, tetrahydrothienyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, tetrahydrothienyl S-oxide, tetrahydrothienyl S,S-dioxide and homothiomorpholinyl S-oxide. The heterocycle groups herein may be substituted in one or more substitutable positions with various groups. For example, such heterocycle groups may be optionally substituted with C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl, di(C₁-C₆)alkylamino(C₁-C₆)alkyl or ═O.

Any term that includes two radicals such as, for example, “arylalkyl”, denotes the first radical, or aryl as in the example, attached to the concluding radical, or alkyl as in the example. The concluding radical is attached to the substituent in question. Compounds of the present invention can possess, in general, one or more asymmetric carbon atoms and are thus capable of existing in the form of optical isomers as well as in the form of racemic or non-racemic mixtures thereof. Unless otherwise indicated, the compounds of the present invention, as depicted or named, may exist as the racemate, a single enantiomer, or any uneven (i.e. non 50/50) mixture of enantiomers. The optical isomers can be obtained by resolution of the racemic mixtures according to conventional processes, e.g., by formation of diastereoisomeric salts, by treatment with an optically active acid or base. Examples of appropriate acids are tartaric, diacetyltartaric, dibenzoyltartaric, ditoluoyltartaric, and camphorsulfonic acid and then separation of the mixture of diastereoisomers by crystallization followed by liberation of the optically active bases from these salts. A different process for separation of optical isomers involves the use of a chiral chromatography column, such as, for example, a CHIRAL-AGP column, optimally chosen to maximize the separation of the enantiomers. Still another available method involves synthesis of covalent diastereoisomeric molecules by reacting compounds of the invention with an optically pure acid in an activated form or an optically pure isocyanate. The synthesized diastereoisomers can be separated by conventional means such as chromatography, distillation, crystallization or sublimation, and then hydrolyzed to deliver the enantiomerically pure compound. The optically active compounds of the invention can likewise be obtained by using optically active starting materials. These isomers may be in the form of a free acid, a free base, an ester or a salt.

The compounds of the invention can be synthesized by procedures known in the art and by the procedures depicted in Schemes found below.

Non-limiting examples of specific tumor types that the compounds may be used to treat include, but are not limited to sarcomas, melanomas, neuroblastomas, carcinomas (including but not limited to lung, renal cell, ovarian, liver, bladder, and pancreatic carcinomas), and mesotheliomas.

As used herein, the term “amount effective” means a dosage sufficient to produce a desired result. The desired result can be subjective or objective improvement in the recipient of the dosage; a decrease in tumor size, time to progression of disease, and/or survival; inhibiting an increase in tumor size; reducing or preventing metastases; and/or limiting or preventing recurrence of the tumor in a subject that has previously had a tumor.

In one embodiment, the methods of the invention can be used in combination with surgery on the subject, wherein surgery includes primary surgery for removing one or more tumors, secondary cytoreductive surgery, and palliative secondary surgery.

In a further embodiment, the methods of the invention further comprise treating the subject with chemotherapy and/or radiation therapy. One benefit of such a method if that use of the compounds permits a reduction in the chemotherapy and/or radiation dosage necessary to inhibit tumor growth and/or metastasis. As used herein, “radiotherapy” includes but is not limited to the use of radio-labeled compounds targeting tumor cells. Any reduction in chemotherapeutic or radiation dosage benefits the patient by resulting in fewer and decreased side effects relative to standard chemotherapy and/or radiation therapy treatment. In this embodiment, the one or more compounds may be administered prior to, at the time of, or shortly after a given round of treatment with chemotherapeutic and/or radiation therapy. In a preferred embodiment, the one or more compounds is administered prior to or simultaneously with a given round of chemotherapy and/or radiation therapy. In a most preferred embodiment, the one or more compounds is administered prior to or simultaneously with each round of chemotherapy and/or radiation therapy. The exact timing of compound administration will be determined by an attending physician based on a number of factors, but the compound is generally administered between 24 hours before a given round of chemotherapy and/or radiation therapy and simultaneously with a given round of chemotherapy and/or radiation therapy.

The methods of the invention are appropriate for use with chemotherapy using one or more cytotoxic agent (ie: chemotherapeutic), including, but not limited to, cyclophosphamide, taxol, 5-fluorouracil, adriamycin, cisplatinum, methotrexate, cytosine arabinoside, mitomycin C, prednisone, vindesine, carbaplatinum, and vincristine. The cytotoxic agent can also be an antiviral compound which is capable of destroying proliferating cells. For a general discussion of cytotoxic agents used in chemotherapy, see Sathe, M. et al., Cancer Chemotherapeutic Agents: Handbook of Clinical Data (1978), hereby incorporated by reference. When administered as a combination, the therapeutic agents can be formulated as separate compositions that are given at the same time or different times, or the therapeutic agents can be given as a single composition.

The methods of the invention are also particularly suitable for those patients in need of repeated or high doses of chemotherapy and/or radiation therapy.

The actual compound dosage range for administration is based on a variety of factors, including the age, weight, sex, medical condition of the individual, the severity of the condition, and the route of administration. Thus, the dosage regimen may vary widely, but can be determined by a physician using standard methods. An effective amount of the one or more compounds that can be employed ranges generally between 0.01 μg/kg body weight and 10 mg/kg body weight, preferably ranging between 0.05 μg/kg and 5 mg/kg body weight, more preferably between 1 μg/kg and 5 mg/kg body weight, and even more preferably between about 10 μg/kg and 5 mg/kg body weight.

The compounds may be made up in a solid form (including granules, powders or suppositories) or in a liquid form (e.g., solutions, suspensions, or emulsions). The compounds of the invention may be applied in a variety of solutions and may be subjected to conventional pharmaceutical operations such as sterilization and/or may contain conventional adjuvants, such as preservatives, stabilizers, wetting agents, emulsifiers, buffers etc. The compounds of the invention may be administered by any suitable route, including orally, parentally, by inhalation or rectally in dosage unit formulations containing conventional pharmaceutically acceptable carriers, adjuvants, and vehicles, including liposomes. The term parenteral as used herein includes, subcutaneous, intravenous, intraarterial, intramuscular, intrasternal, intratendinous, intraspinal, intracranial, intrathoracic, infusion techniques, intracavity, or intraperitoneally.

In yet further aspects, the invention provides an article of manufacture comprising packaging material and the above pharmaceutical compositions.

The instant invention may be embodied in other forms or carried out in other ways without departing from the spirit or essential characteristics thereof. The present disclosure and enumerated examples are therefore to be considered as in all respects illustrative and not restrictive, and all equivalency are intended to be embraced therein. One of ordinary skill in the art would be able to recognize equivalent embodiments of the instant invention, and be able to practice such embodiments using the teaching of the instant disclosure and only routine experimentation.

### EXAMPLES

### Example 1

### Synthesis

### A. tert-butyl 2-amino-5,6,7,8-tetrahydro-4H-cyclohepta[b]thiophene-3-carboxylate (3)

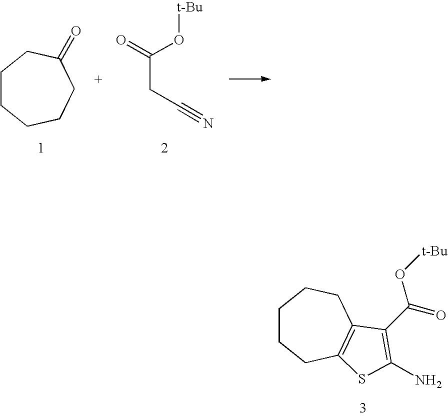

A mixture of 1, 2 and sulfur in EtOH was heated to 45° C. under a nitrogen atmosphere. Morpholine was added dropwise and the reaction mixture was stirred overnight. The mixture was allowed to cool to room temperature and 300 ml of a 0.4 M HOAc-solution was added. The resulting mixture was stirred for 5 minutes and extracted with 3×250 ml of diethylether. The combined organic layers were washed with 3×250-m of water, 200 ml of brine, dried over MgSO₄ and evaporated to dryness. This gave an orange oil which was identified by 1H-NMR as 3. The yield of the pure material was 18.42 g (68.89 mmol, 97%). ¹H NMR (CDCl₃) δ 2.28 (s, 3H), 4.51 (s, 2H), 7.75 (m, 2H), 7.85 (m, 2H). NMR ID-nr (TU, CDCl₃).

### B. tert-butyl 2-(2,2,2-trifluoroacetamido)-5,6,7,8-tetrahydro-4H-cyclohepta[b]thiophene-3-carboxylate (5)

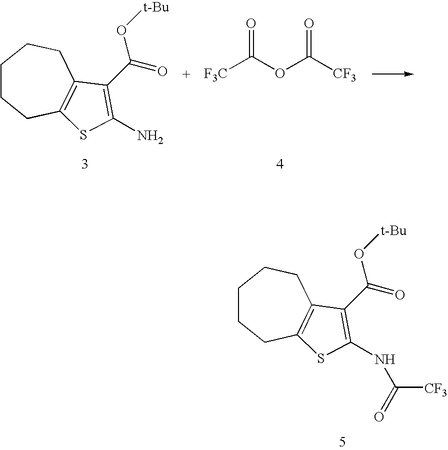

To a solution of 3 and 4 in CH₂Cl₂ cooled to 0° C. was added dropwise N,N-diisopropylethylamine at room temperature under a nitrogen atmosphere. The reaction was stirred for 1 hour and the ice bath was removed. 0.1 Equivalents of 4 (0.96 ml) was added and the reaction was stirred overnight after which TLC showed 90% of conversion of 3. The mixture was poured onto 300 ml of cold water and the layers were separated. The organic layer was washed with 2×250 ml of 1 M HCl, 2×250 ml of water and 150 ml of brine, dried over MgSO₄ and the solvent evaporated to dryness. The resulting orange solid was purified by column over silica gel (13×4.5 cm) using 1% EtOAc/PE as the solvent mixture in 8 g batches to give an off-white solid which was identified by 1H-NMR as 5. The yield of the pure material was 17.79 g (48.95 mmol, 71%). ¹H NMR (CDCl₃) δ 2.28 (s, 3H), 4.51 (s, 2H), 7.75 (m, 2H), 7.85 (m, 2H). NMR ID-nr (TU, CDCl₃).

### C. 2-(2,2,2-trifluoroacetamido)-5,6,7,8-tetrahydro-4H-cyclohepta[b]thiophene-3-carboxylic Acid (7)

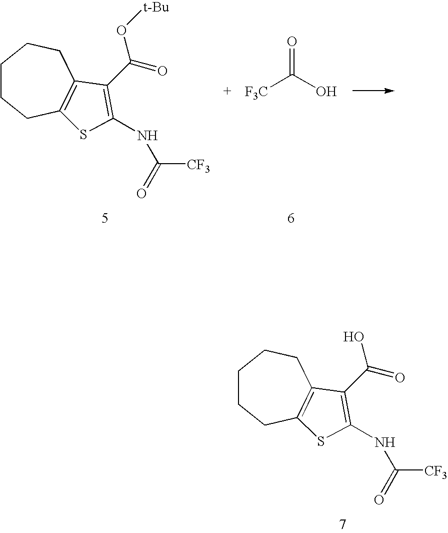

To a solution of 5 in CH₂Cl₂ was added dropwise 6 at room temperature under a nitrogen atmosphere and the resulting reaction mixture was stirred for 4 h. The volatile materials were removed in vacuo and the residue was taken up in MeOH and evaporated to azeotropically remove any traces of 6. The remaining solids were washed with hot hexane and dried in vacuo yielding a white powder which was identified by 1H-NMR as 7. The yield of the pure material was 4.92 g (16.01 mmol, 97%). ¹H NMR (CDCl₃) δ 2.28 (s, 3H), 4.51 (s, 2H), 7.75 (m, 2H), 7.85 (m, 2H). NMR ID-nr (TU, CDCl₃).

### D. 2-(2,2,2-trifluoroacetamido)-N-(3-chlorophenyl)-5,6,7,8-tetrahydro-4H-cyclohepta[b]thiophene-3-carboxamide (9)

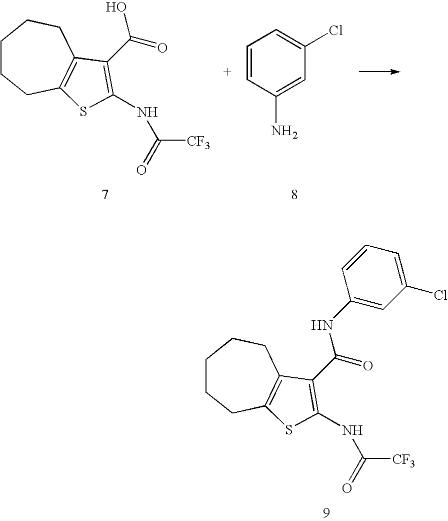

To a solution of EDCI and DMAP in 75 ml of CH₂Cl₂ was added a suspension of 7 and 8 in 50 ml CH₂Cl₂. The reaction mixture was heated overnight at 60° C. The resulting mixture was evaporated to dryness and the residue was partitioned between 300 ml of 1M HCl-solution and 300 ml of EtOAc. The organic layer was washed with sequentially with water, sat. NaHCO₃-solution, water and brine. The organic layer was then dried with MgSO₄ and evaporated to dryness to afford a white solid. 250 ml of MeOH was added and stirred for 5 minutes. 50 ml of 1M NaOH-solution was then added and the mixture was stirred for 1½ hours at room temperature. The MeOH was removed in vacuo and the pH of the residue was adjusted to 3 with 1M HCl-solution. The desired product was extracted with 3×150 ml of EtOAc, washed with brine and dried with MgSO₄. The organic solution was evaporated to dryness and the resulting solid was washed with diethylether, giving a white powder, which was identified by NMR as 9. The yield of the pure material was 4.92 g (16.01 mmol, 97%). ¹H NMR (CDCl₃) δ 2.28 (s, 3H), 4.51 (s, 2H), 7.75 (m, 2H), 7.85 (m, 2H). NMR ID-nr (TU, CDCl₃).

The other compounds of the invention were prepared in a similar manner as described above.

### Example 1

### Tissue Processing

Excess tissue specimens obtained from organs and tissues such as lung and testicle were obtained freshly at the time of surgery and samples were sent for pathological testing. For diagnosis and grading of tissue samples (ie: prior to processing), hematoxylin and eosin stained tissue sections were examined by a pathologist. If the diagnosis and grading of the tissue concurred with the determination made by the surgical pathologist that provided the tissue, then the tissue was used in the screen. If there was no agreement, then two additional pathologists served as referees. If no consensus was reached, then the tissue was discarded.

The remaining tissue was used to prepare cell suspensions. The tissue was initially treated enzymatically via standard methods until only undigested material remained. The digested cell suspension was filtered through one or more screens of between 40 micron and 100 micron porosity. The resulting cell suspension was further purified via isokinetic density centrifugation.

Additional normal cells were removed from the cell suspension by negative immunoselection with a combination of monoclonal antibodies linked to magnetic beads (Dynal) that were used according to the manufacturers' instructions. The remaining cells were placed into appropriate medium, frozen down in 1.0 mL aliquots, and stored until use.

### Example 2

### General Screen/Bioassay Procedures

After tissue processing, the relative purity of the resulting cell suspension was determined by cytological examination after pap staining. Only those cell preparations greater than 80% tumor cells were used for testing of candidate compounds. If there was any doubt about the percentage of tumor cells in the cell preparation, additional pathologists served as referees to make a determination.

Cell preparations that passed histological and cytological examination for diagnosis, grading, and cell purity were thawed at 37° C. and resuspended in tissue culture medium designed to maintain the cells during the incubation period. The live and dead cells were counted and the cells were diluted in culture medium to 1.0×10³ live cells/test well for tumor cells and 3.3×10³ live cells/test well for normal cells.

The cells were added to microtiter plates and incubated at 37° C. overnight with 10 μM of the candidate compounds that were added at 1/10th the volume of the cell suspension. Alamar Blue (Accumed International, Westlake Ohio) was then added to the cells at 1/10 the volume of the well, and the cells were further incubated at 37° C. for various times. Alamar Blue dye measures cellular re-dox reactions (ie: cellular respiration) whereby a spectral shift occurs upon reduction of the dye. (Excitation 530 nm; emission 590 nm)

The kinetics of cellular re-dox reactions were subsequently measured at various times, for example at 3 hours, 3 days, and 5 days post-dye addition. These measurements, in comparison with control cells (untreated with compound) and media controls (test wells without cells) provide the percent inhibition of cellular mitochondrial respiration as a result of candidate compound treatment, as well as IC₅₀ determinations.

The Alamar Blue data were subsequently confirmed by microscopic observation, and by the use of calcein AM (Molecular Probes, Eugene Oreg.), a cell permeant esterase substrate that measures both esterase activity and cell membrane activity. If the cell is alive, the dye is converted into a fluorogenic substrate by intracellular esterases and is retained by the cell (excitation 485 nm; emission 530 nm). If the cells are dead, the calcein AM rapidly leaks from the cells and is not converted into a fluorogenic substrate. Thus, the assay is useful for cytotoxicity testing.

### Example 3

### Anti-Tumor Screen

In a blinded fashion, approximately 340,000 samples (representing approximately five million compounds) were tested at a rate of 1,000-4,000 compounds per run set against soft tissue sarcoma tumors, while approximately 10,000 of the compounds were also tested against colon and lung tumors. The anti-tumor screen utilized was composed of four tiers as follows. In Screen 1, patient tumor cells were tested in singles, with candidate samples. Samples that showed at least 80% inhibition (compared to cell and media controls) and/or at least two standard deviations from the mean of the plate samples were advanced. In the second test (Screen 2), the compounds were re-tested, in replicate, by serial dilution on patient tumors and the potency (IC₅₀) was determined. Samples that demonstrated nM potency for purified compounds, or microgram/ml potency for natural product extracts, were advanced to the third test (Screen 3). Samples were tested in Screen 3, in a dose-responsive manner, on both patients' tumor cells and normal cells. Samples that were greater than or equal to three times greater potency on tumor cells than normal cells were advanced to fourth test (Screen 4). Compounds were tested in Screen 4 were tested against a wide range of patients' tumor cells of differing anatomical locations and histological origins (sarcomas, melanomas, neuroblastomas, mesotheliomas, and carcinomas including lung, renal, ovarian, liver, bladder, and pancreatic) and normal cells from different anatomical locations (lung, renal, liver, spleen, ovary, peripheral blood mononuclear cells and heart). Those compounds that exhibit greater than, or equal to, three-fold greater potency for the majority of tumor cells rather than normal cells, were advanced for further evaluation and testing.

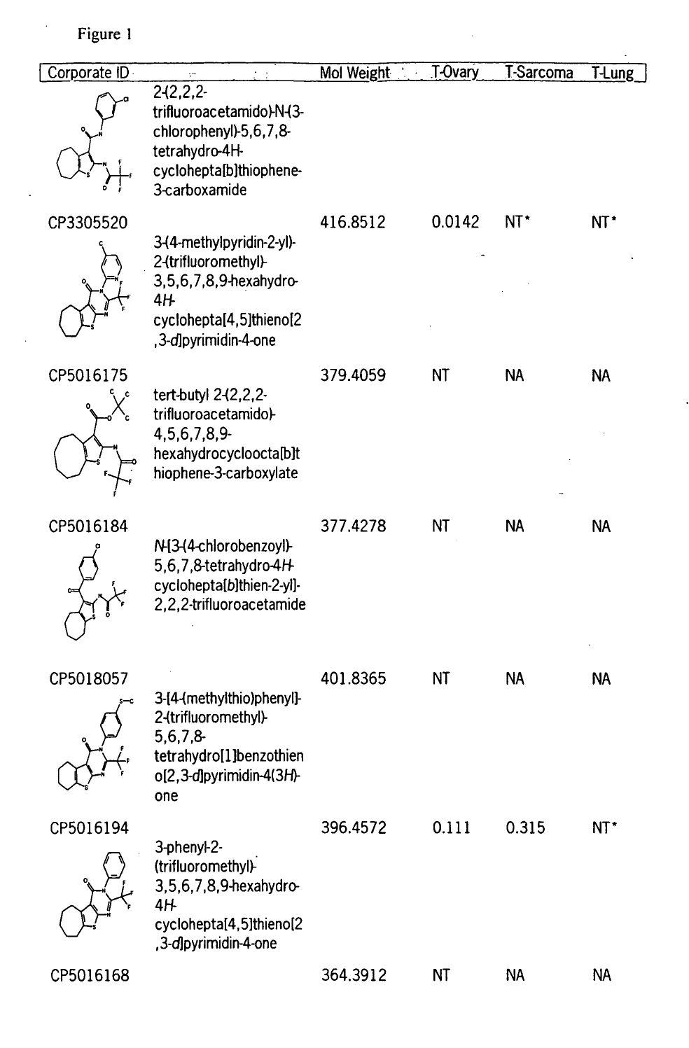

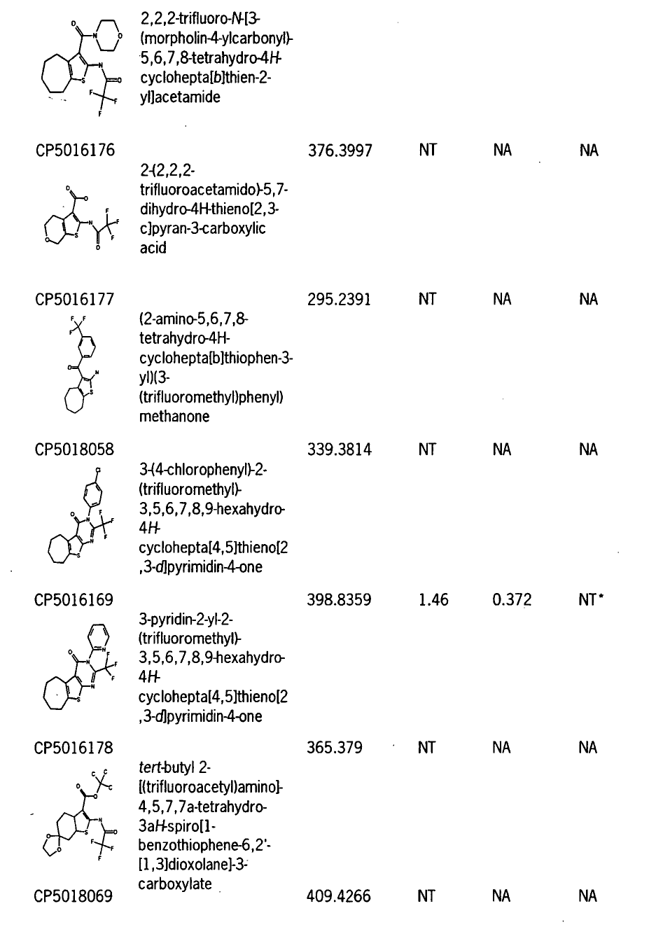

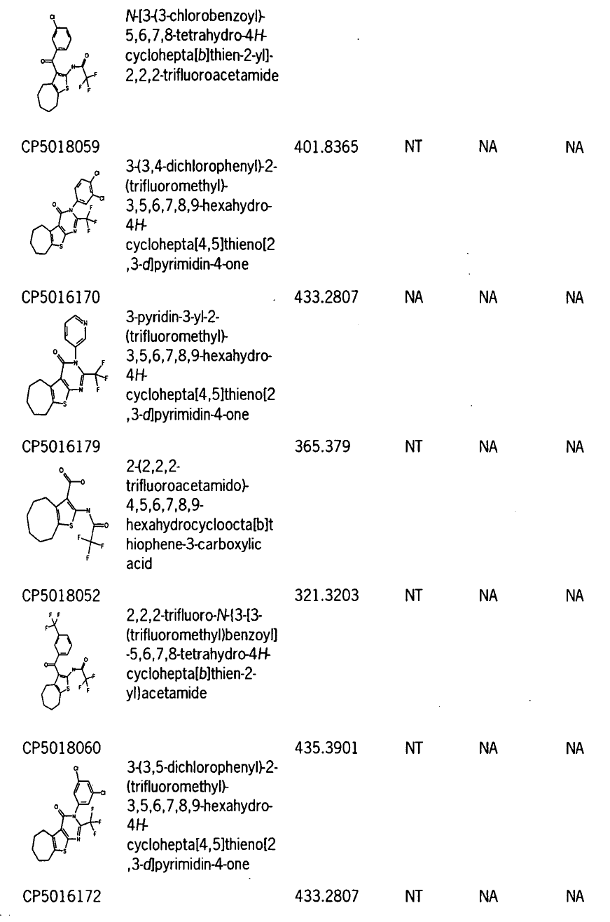

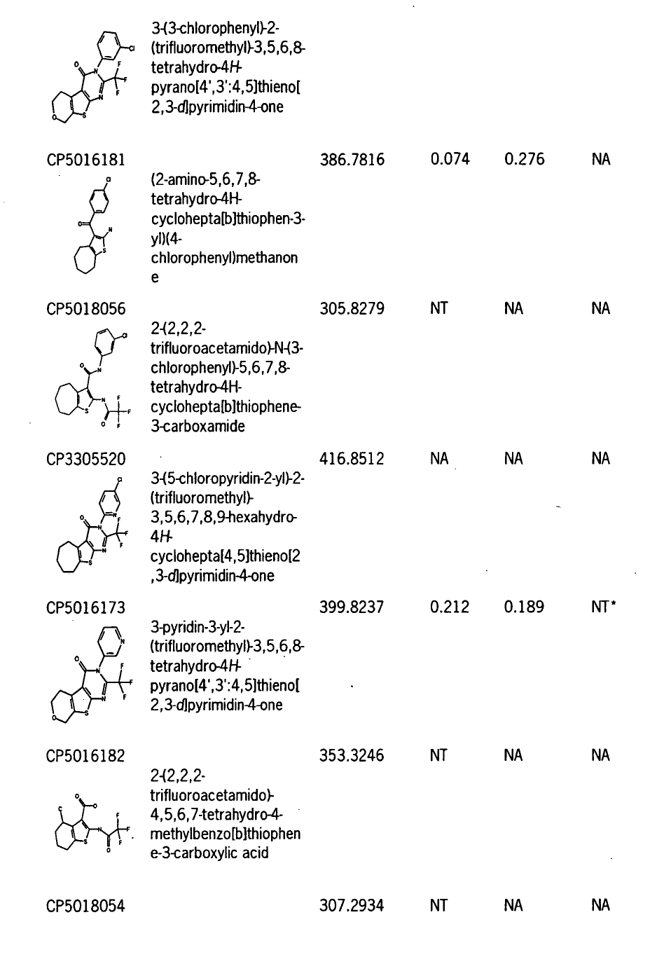

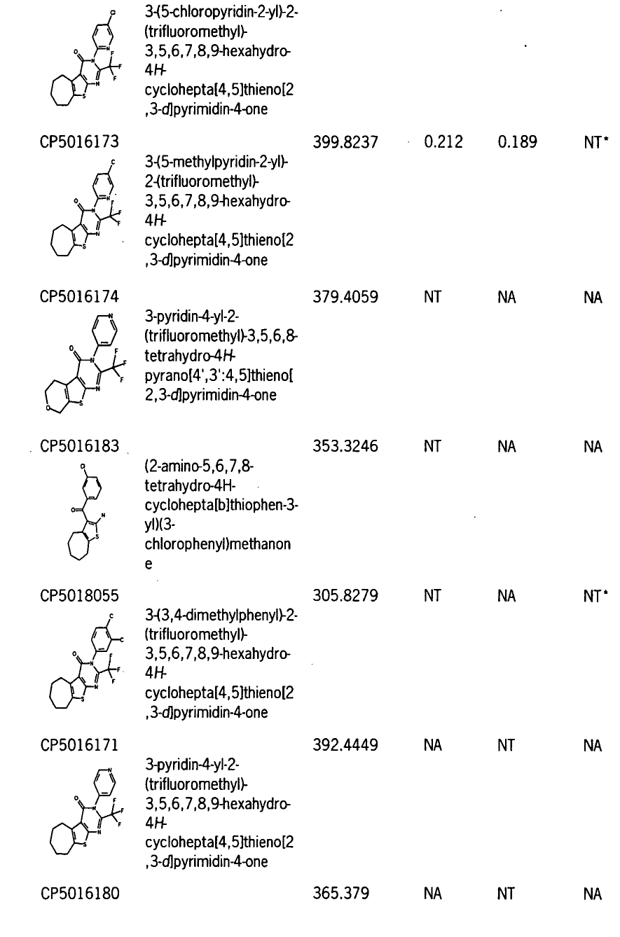

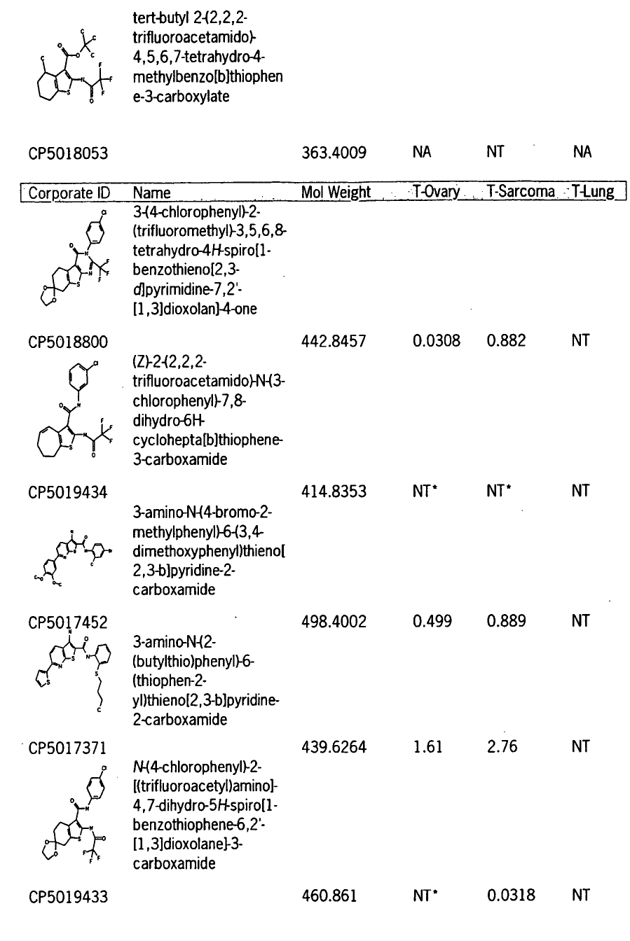

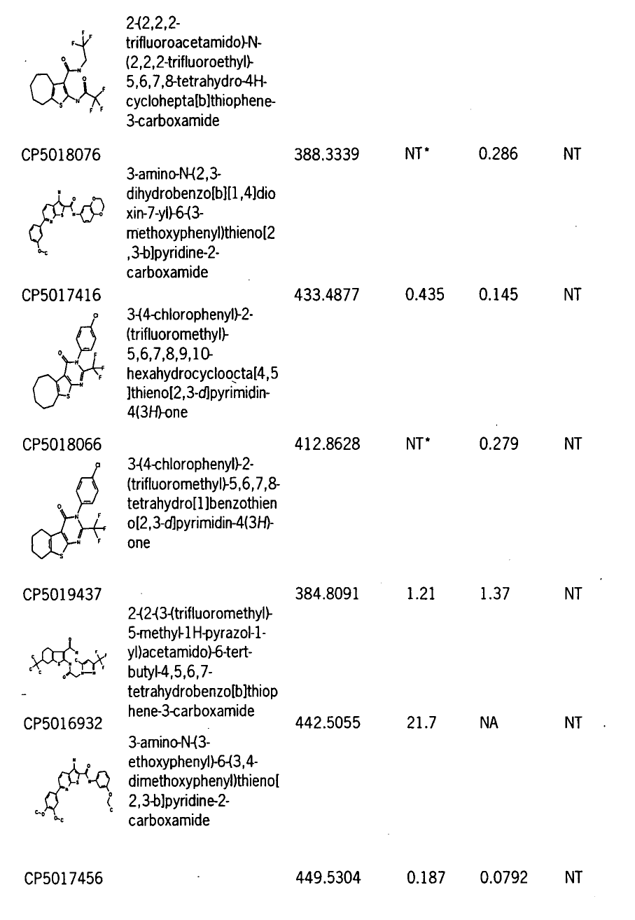

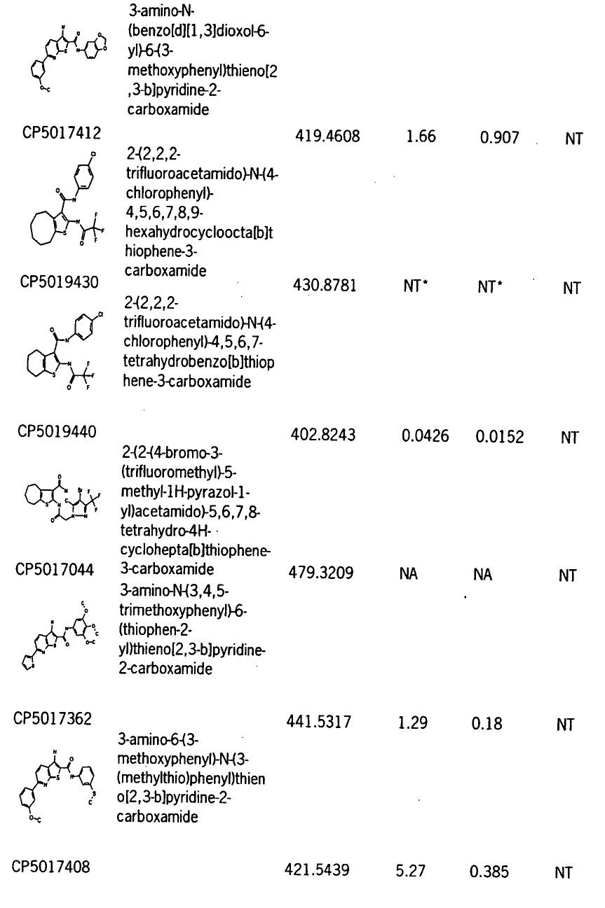

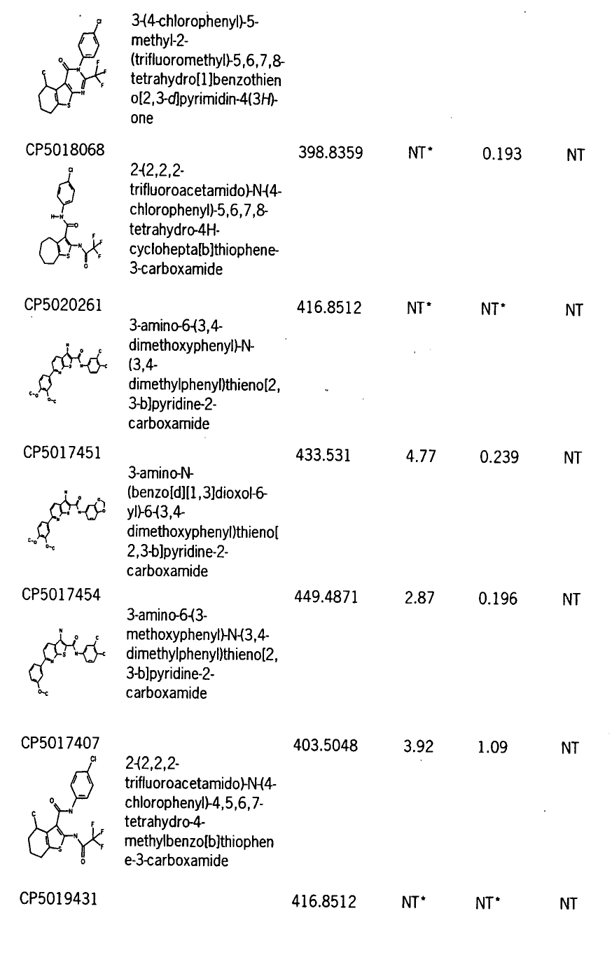

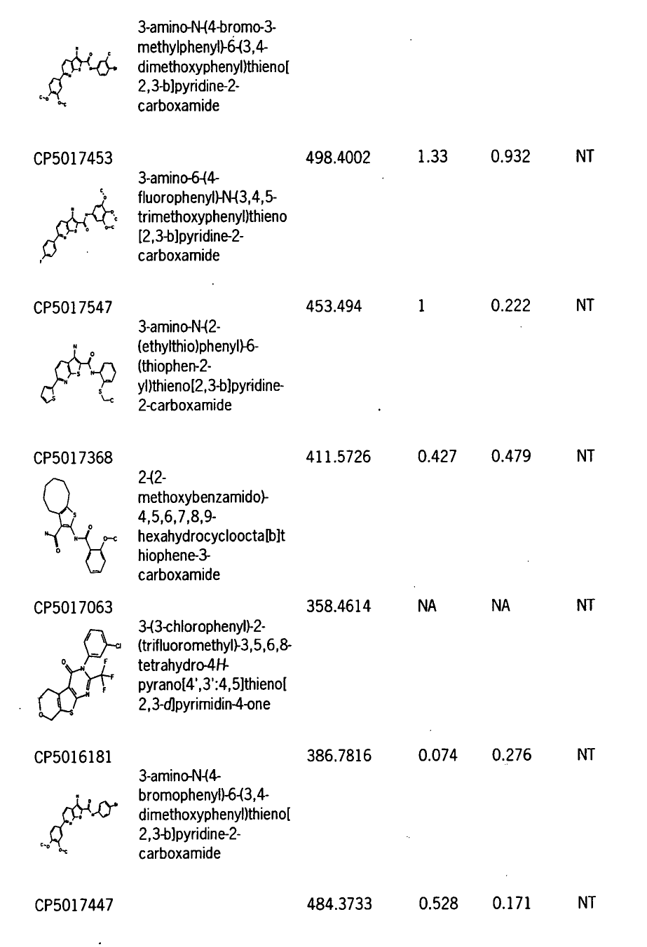

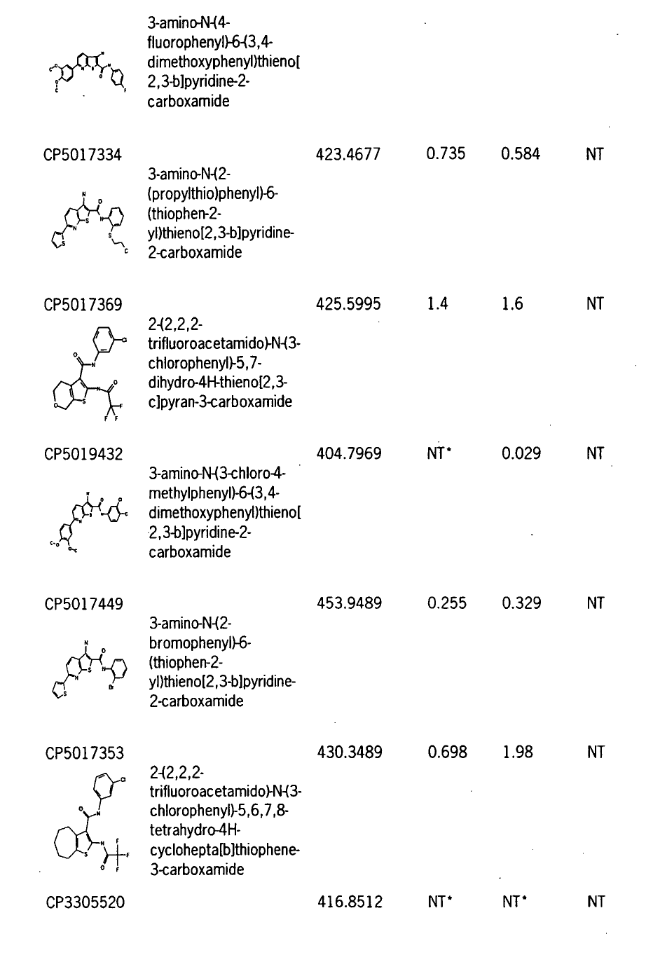

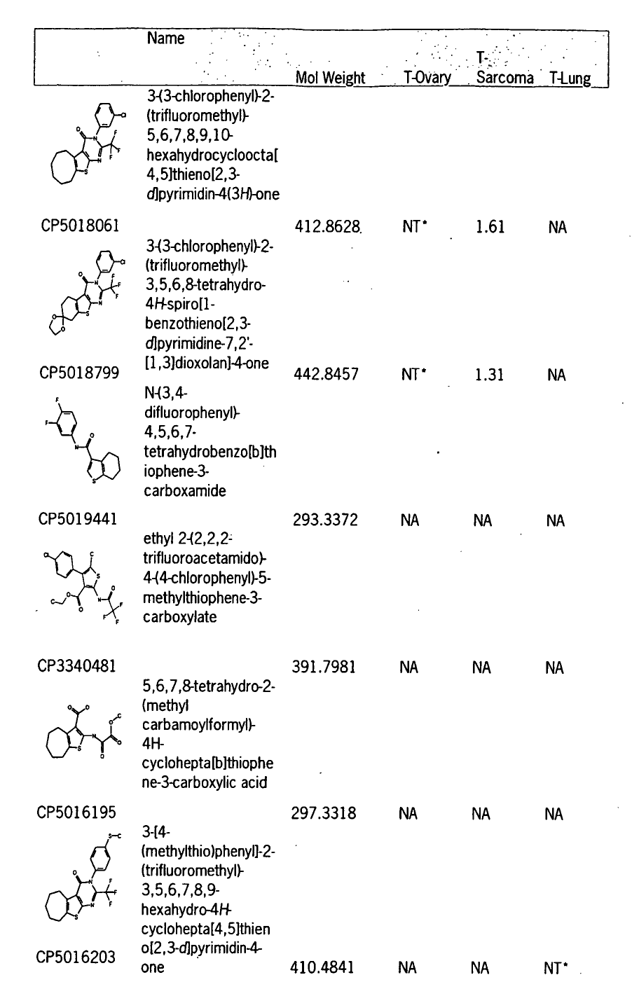

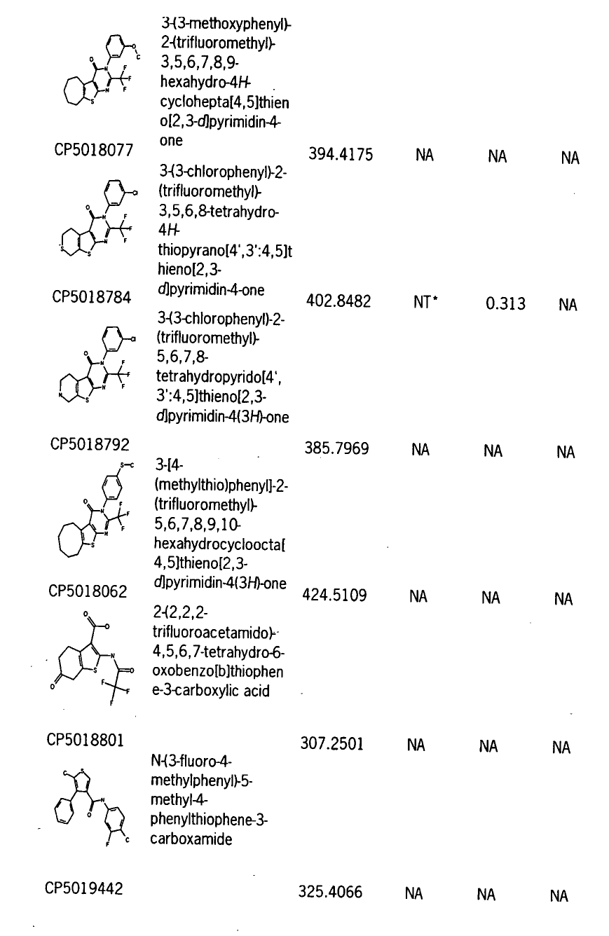

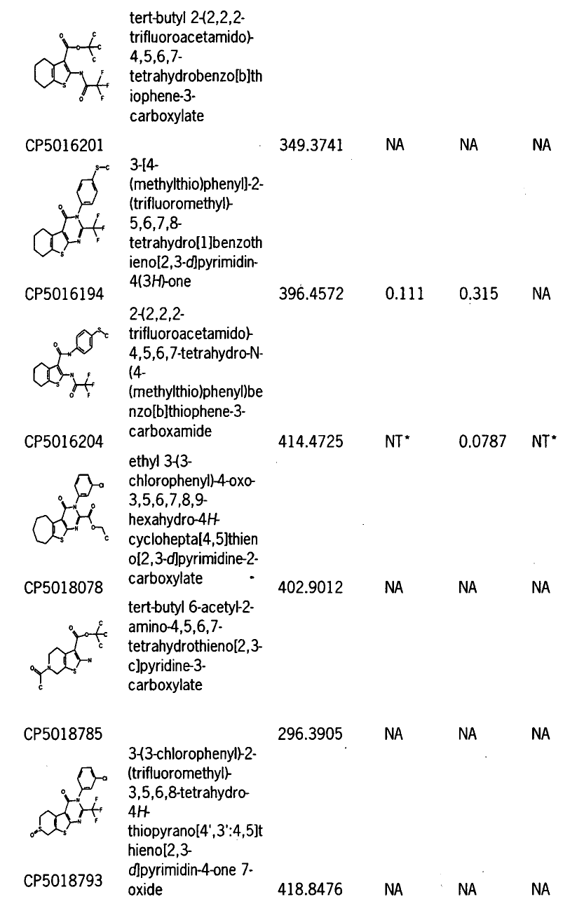

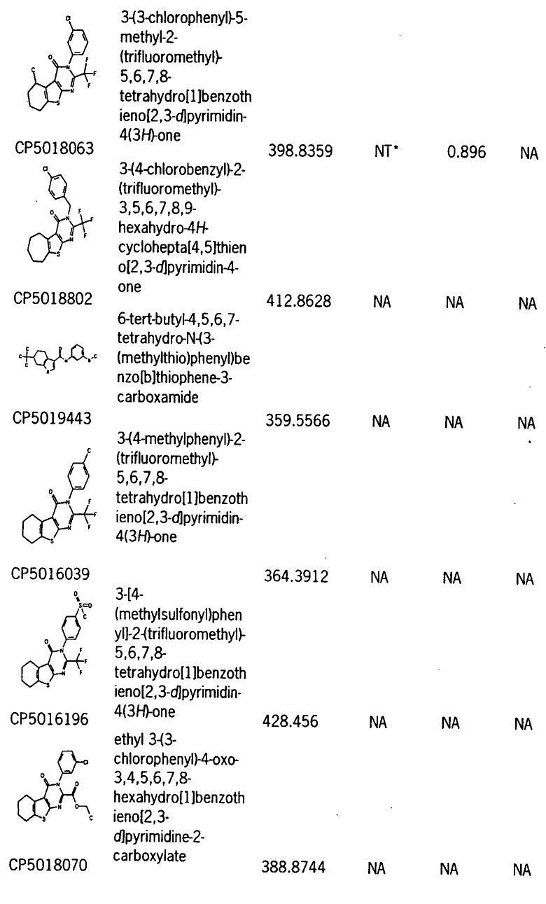

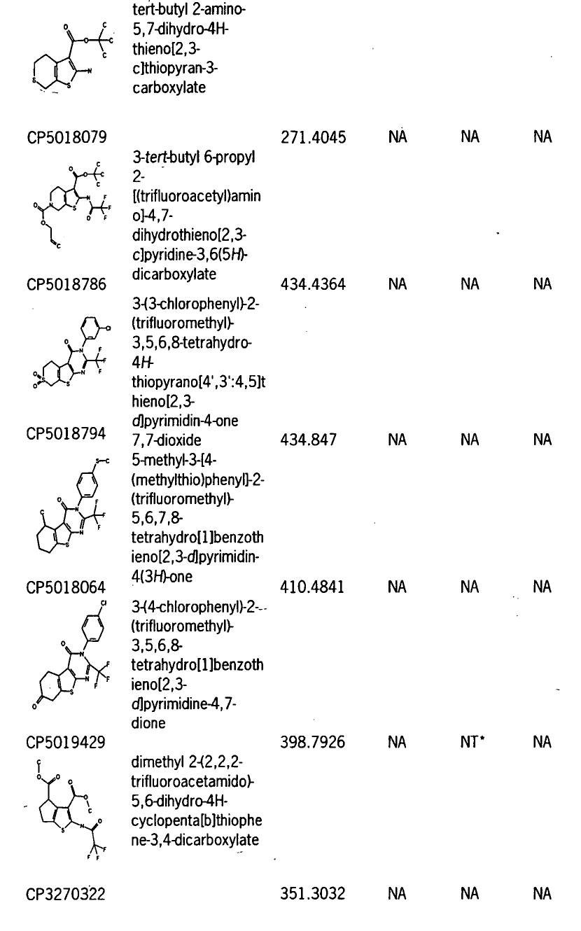

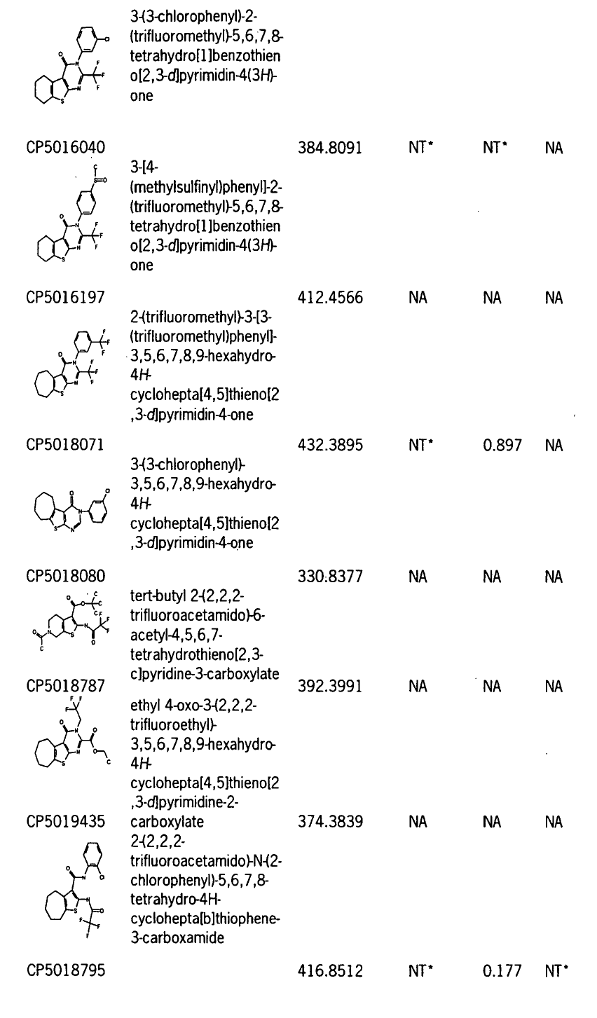

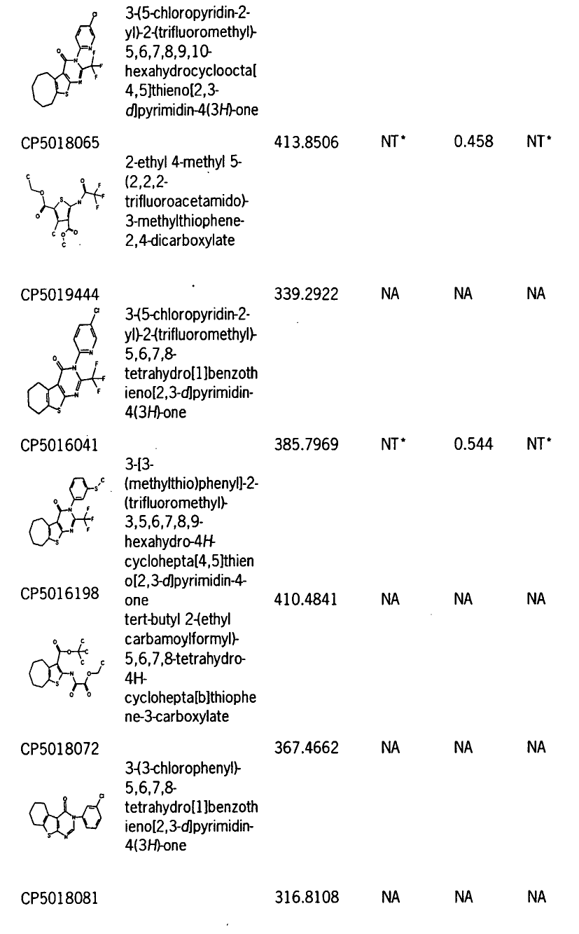

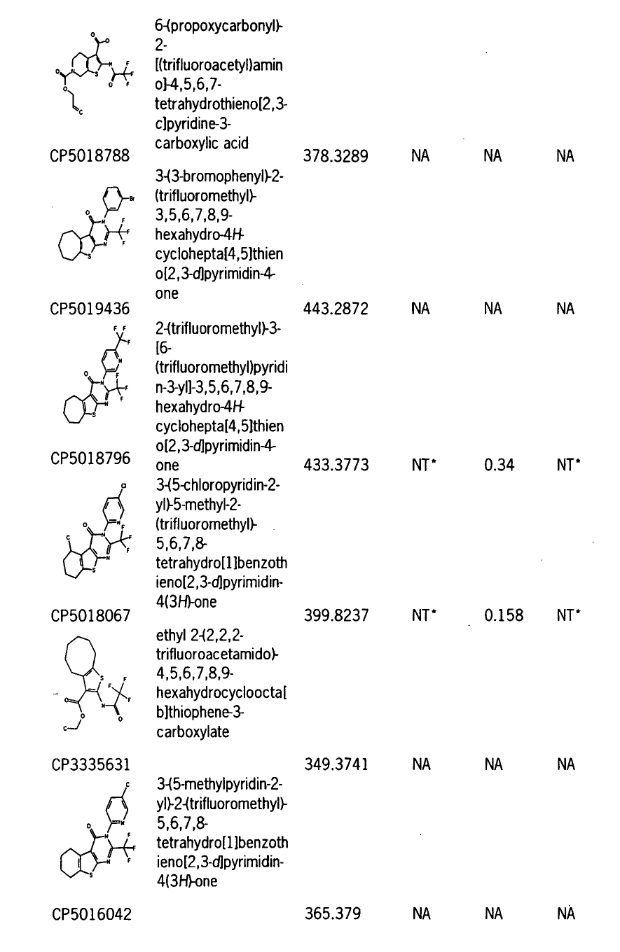

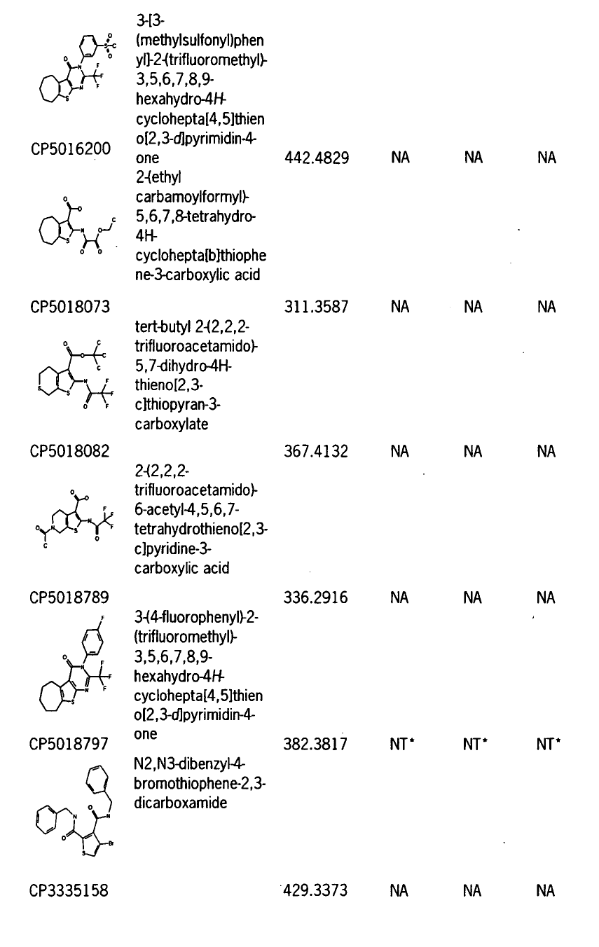

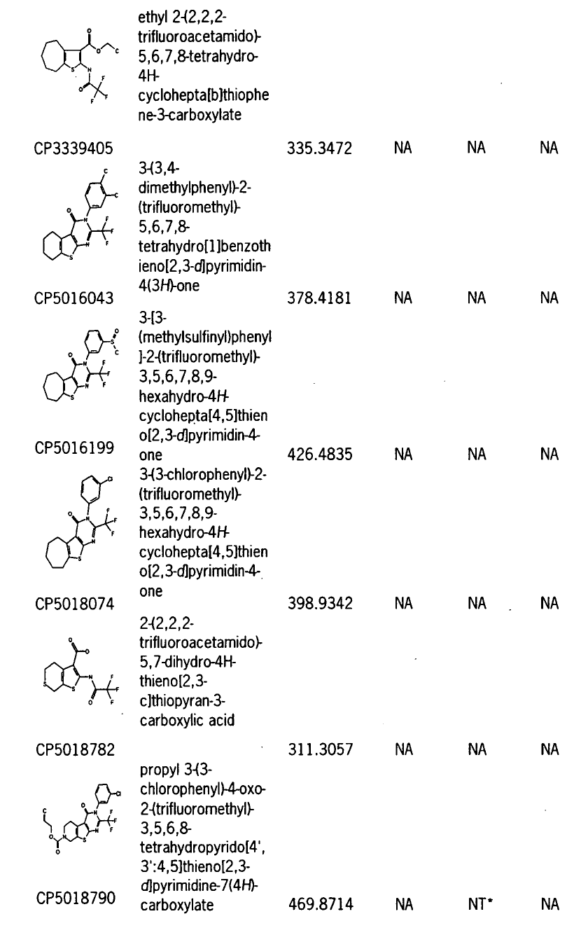

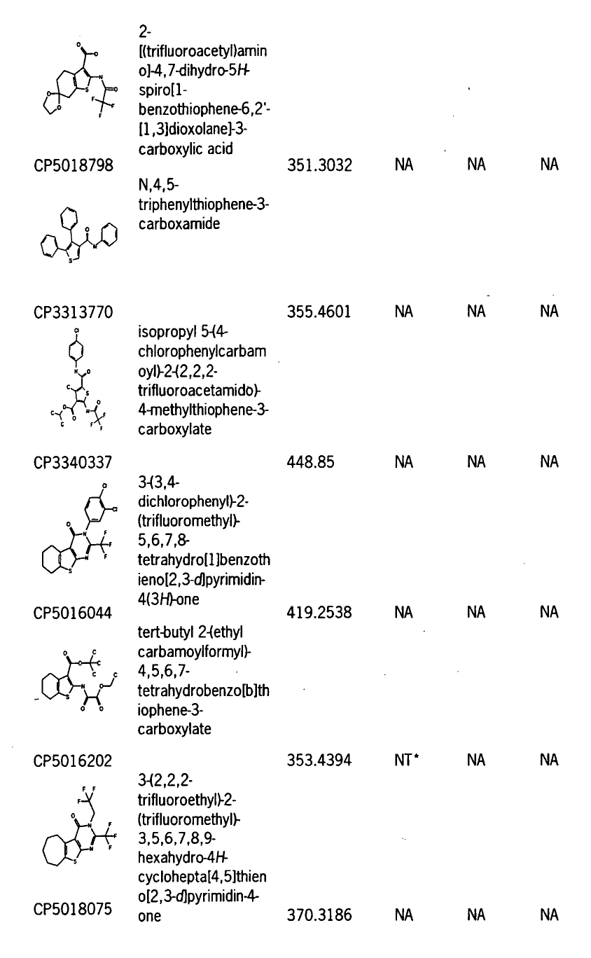

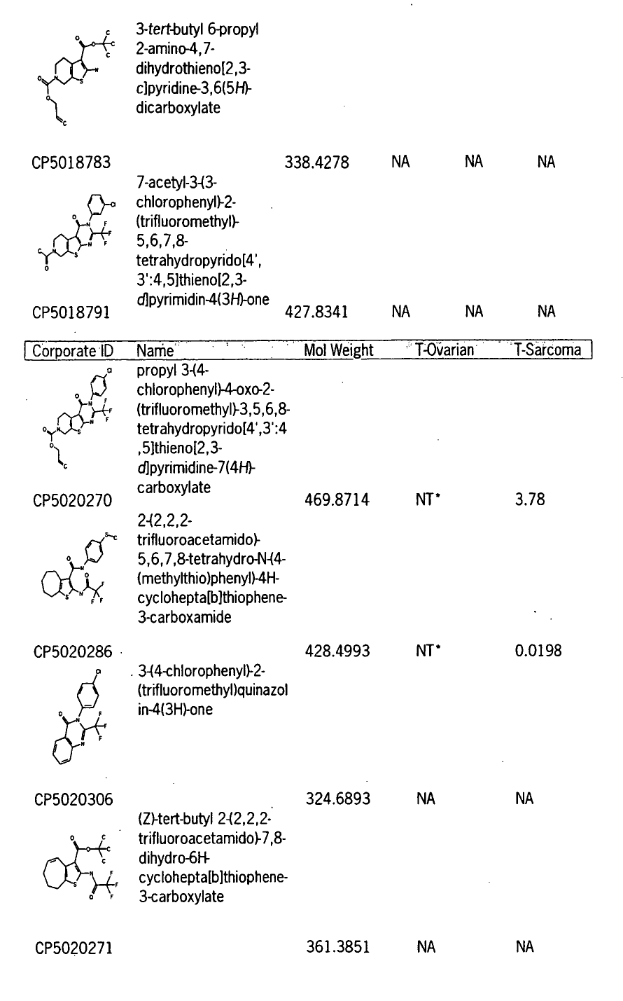

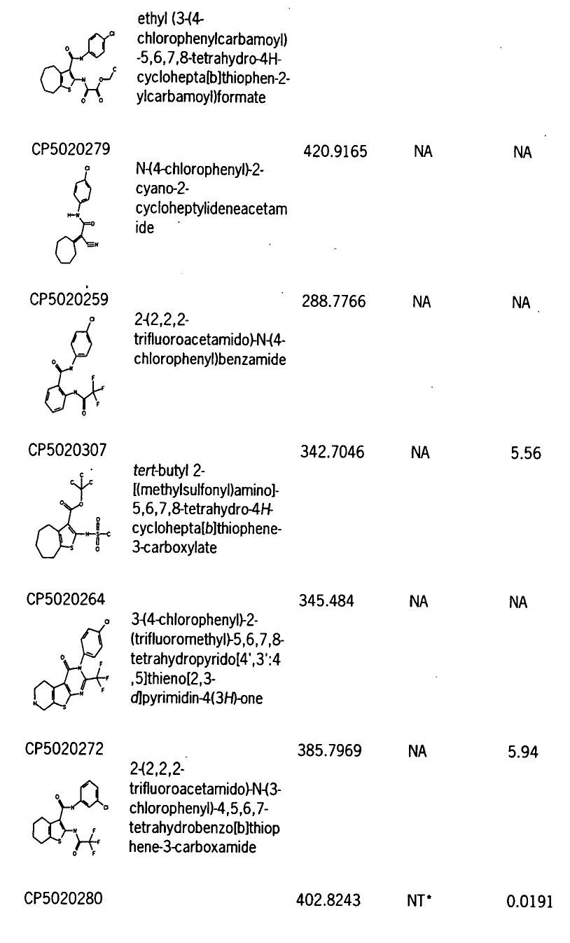

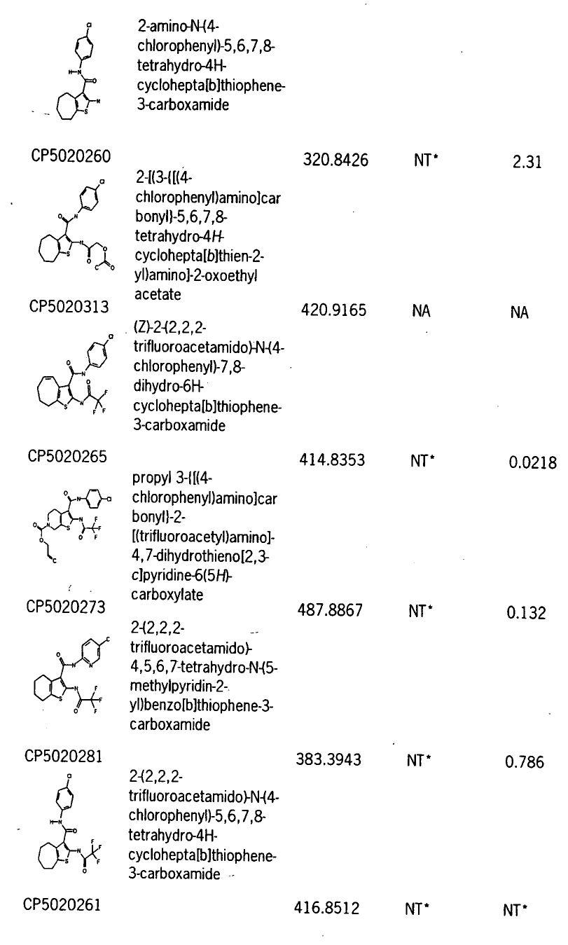

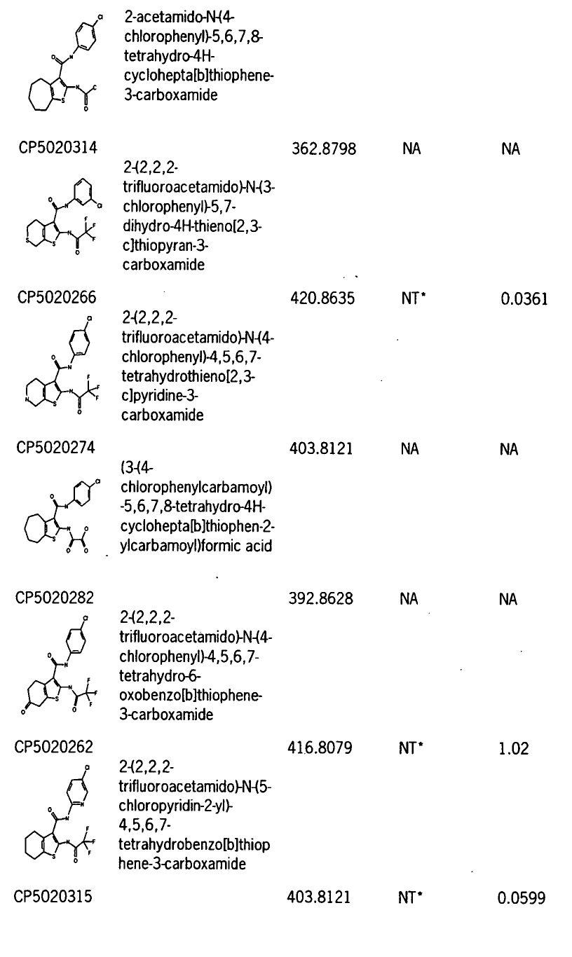

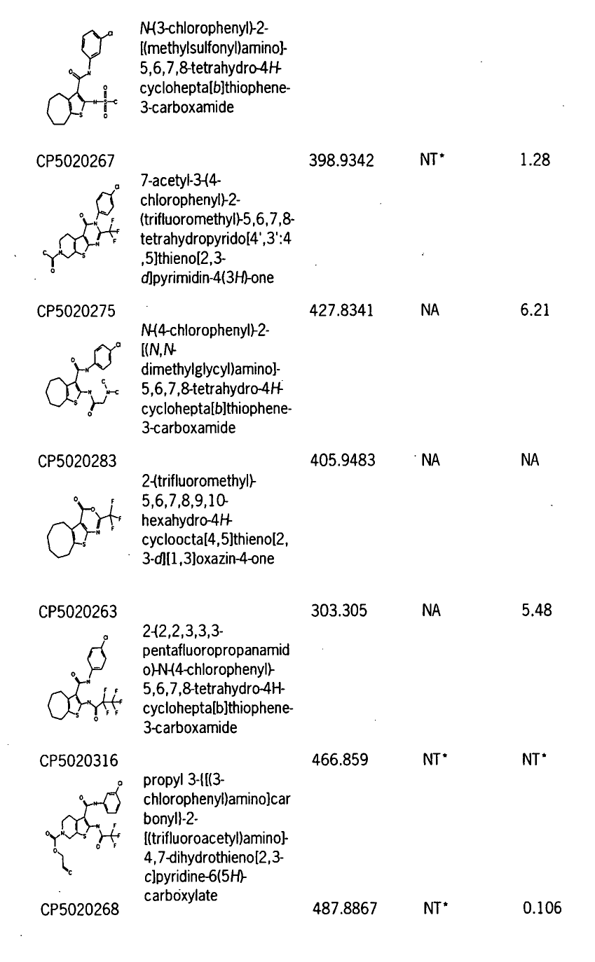

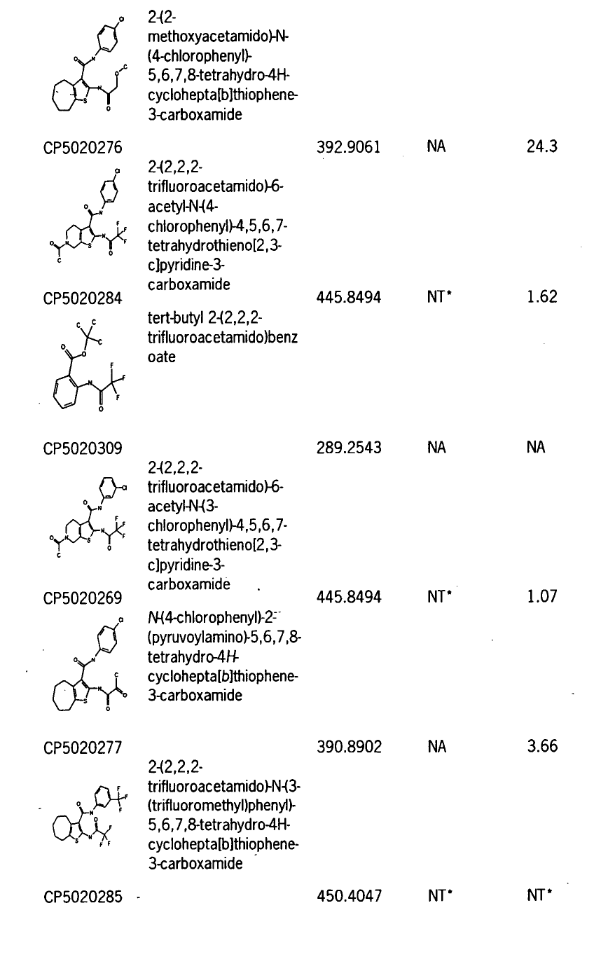

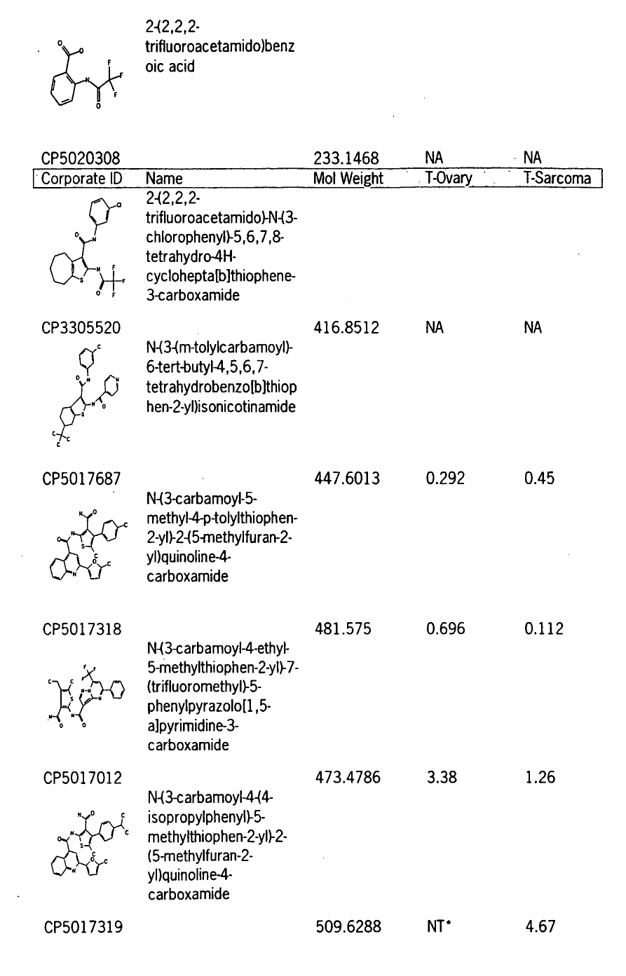

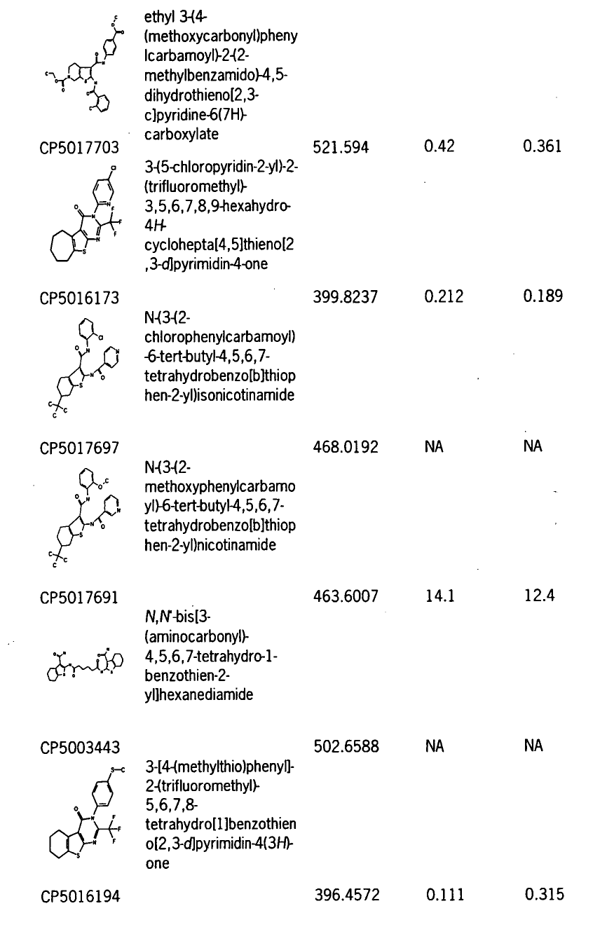

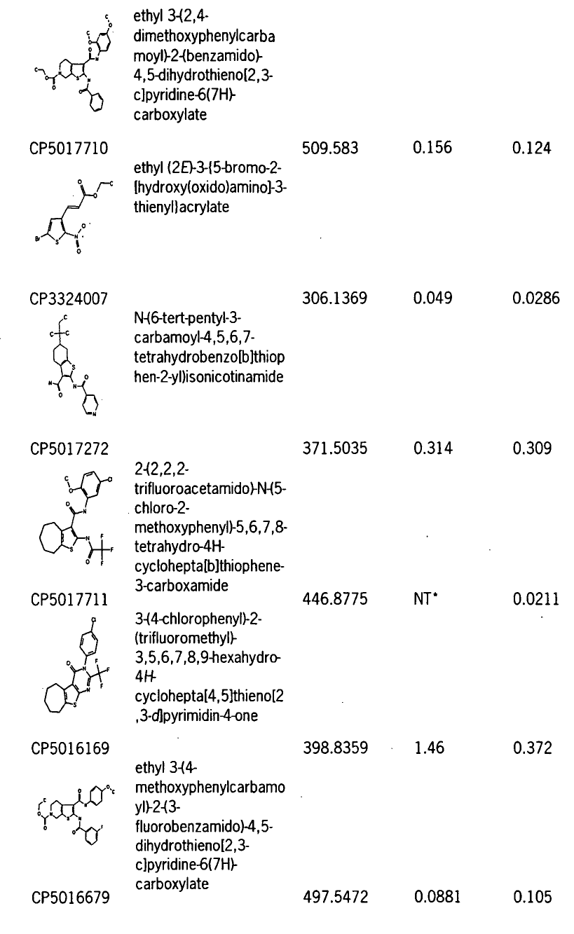

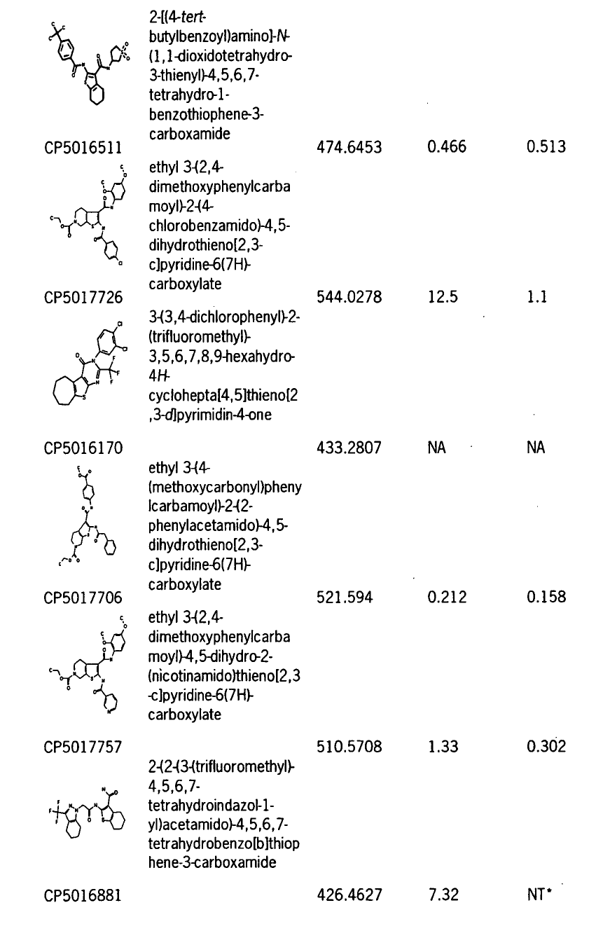

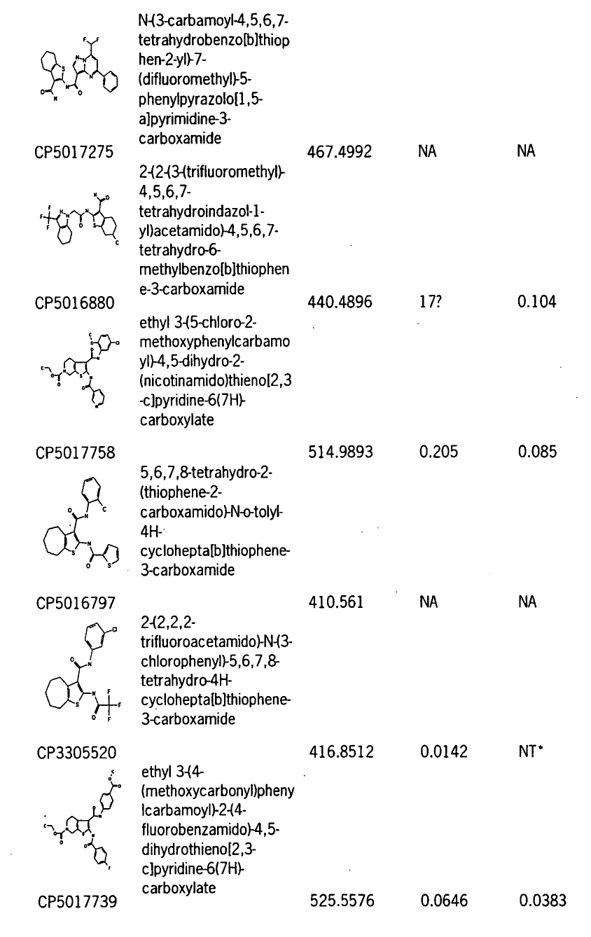

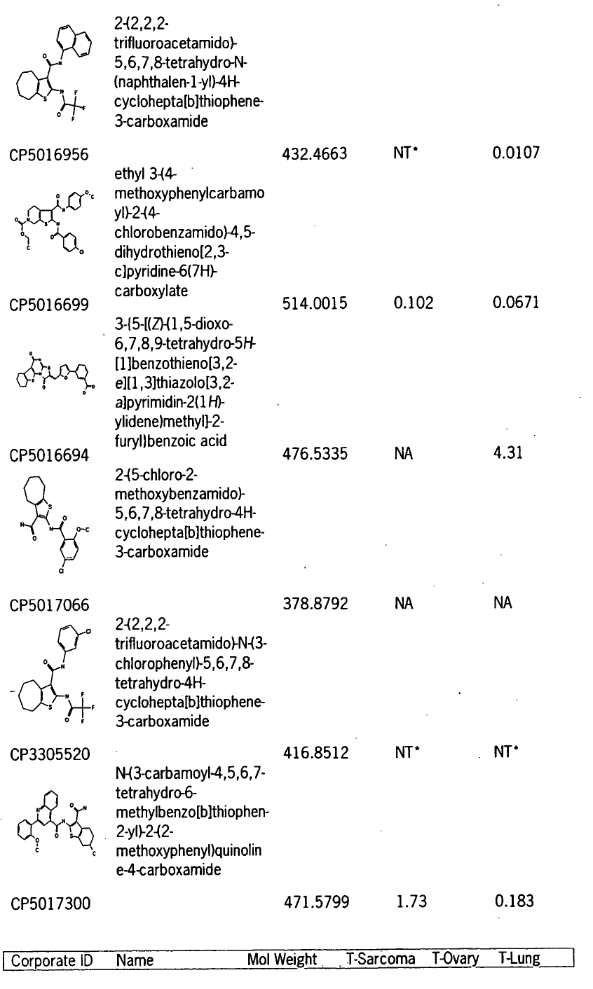

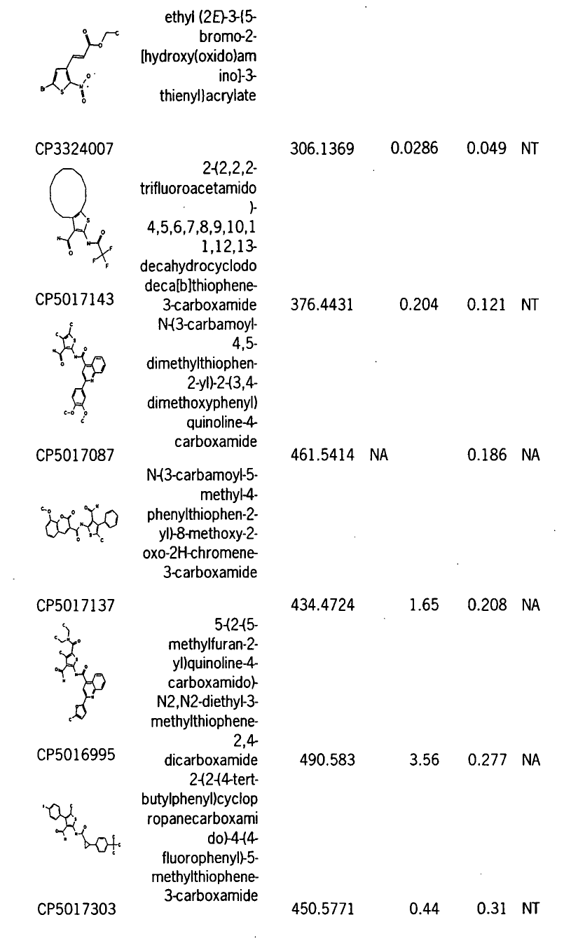

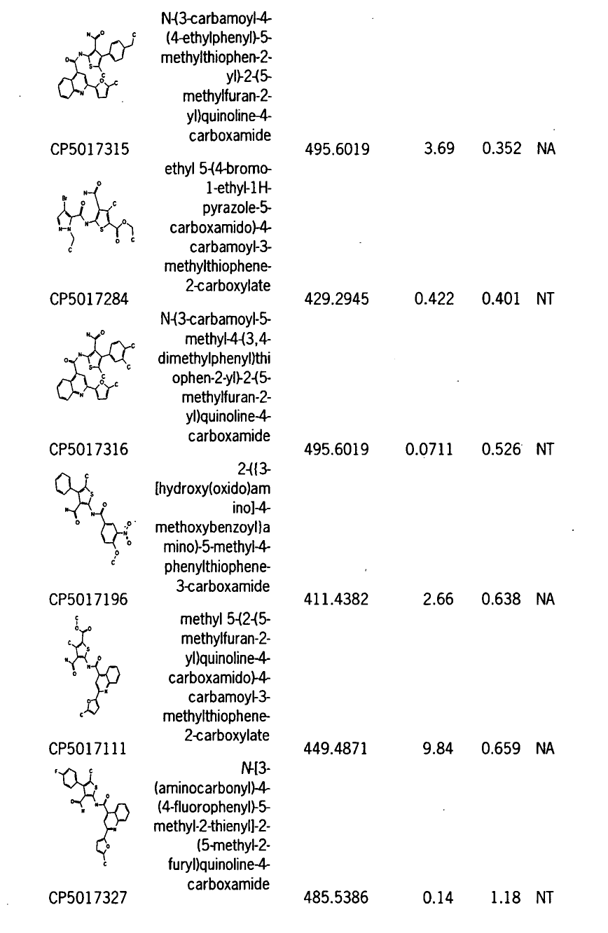

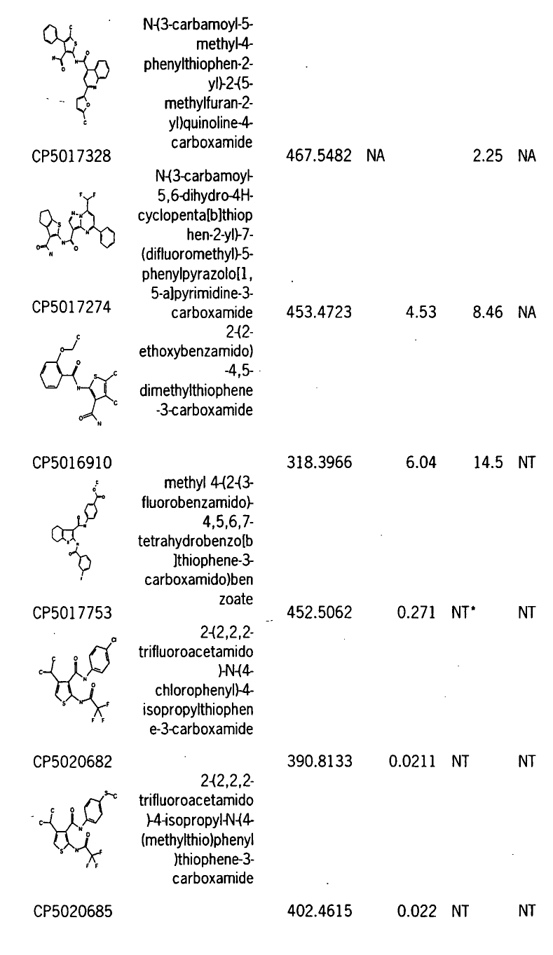

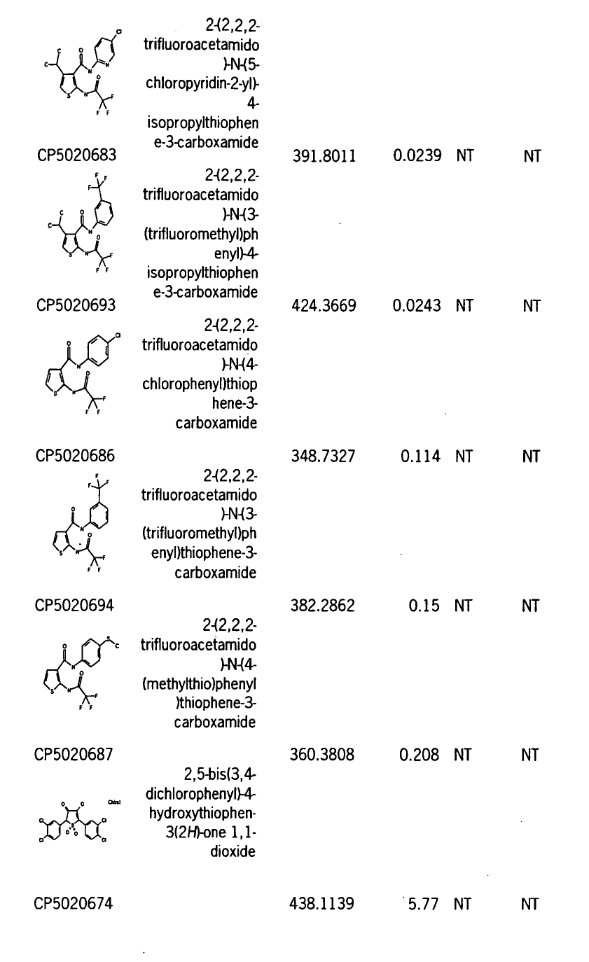

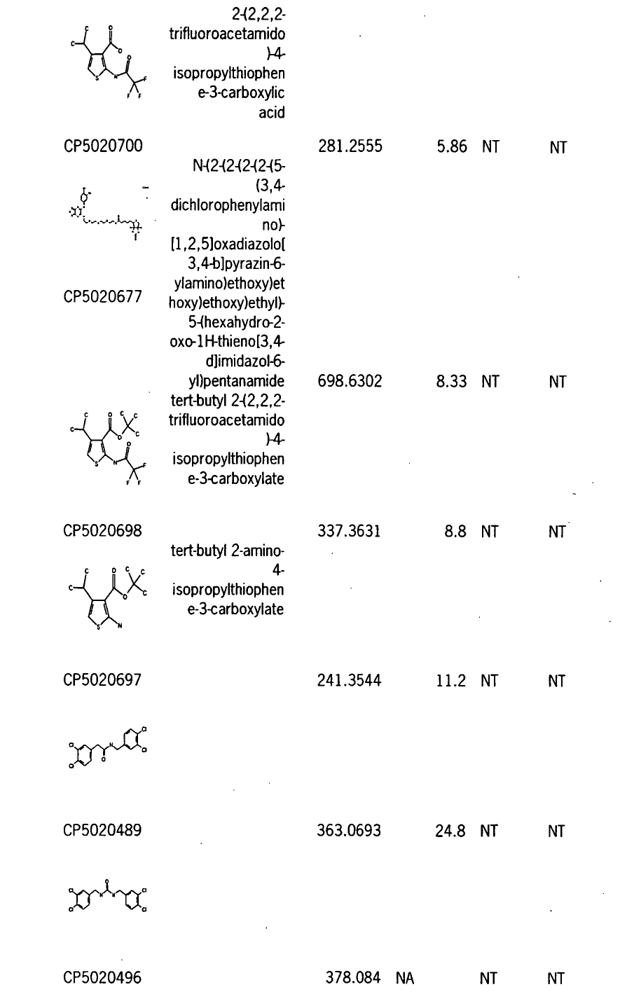

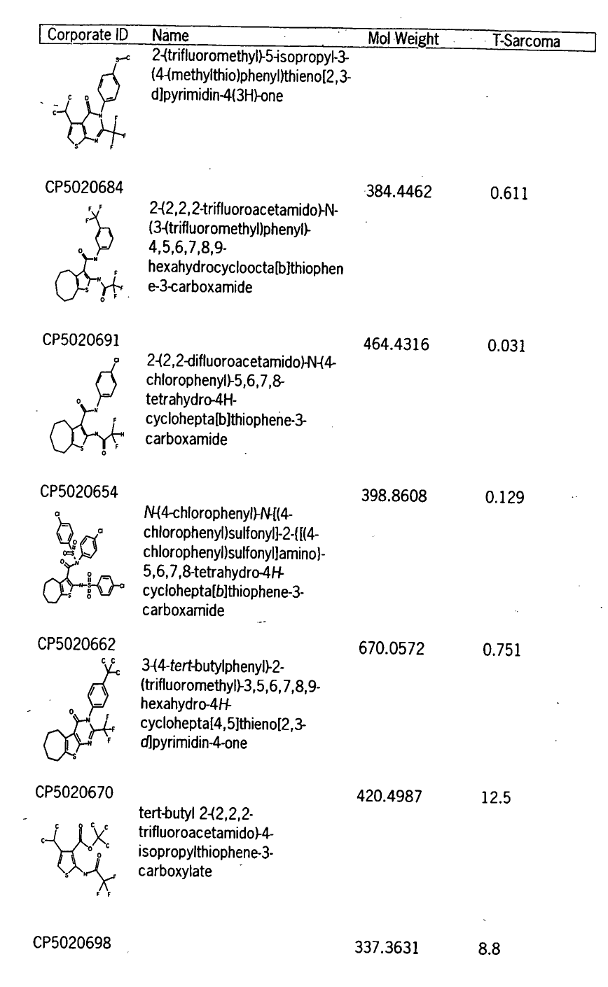

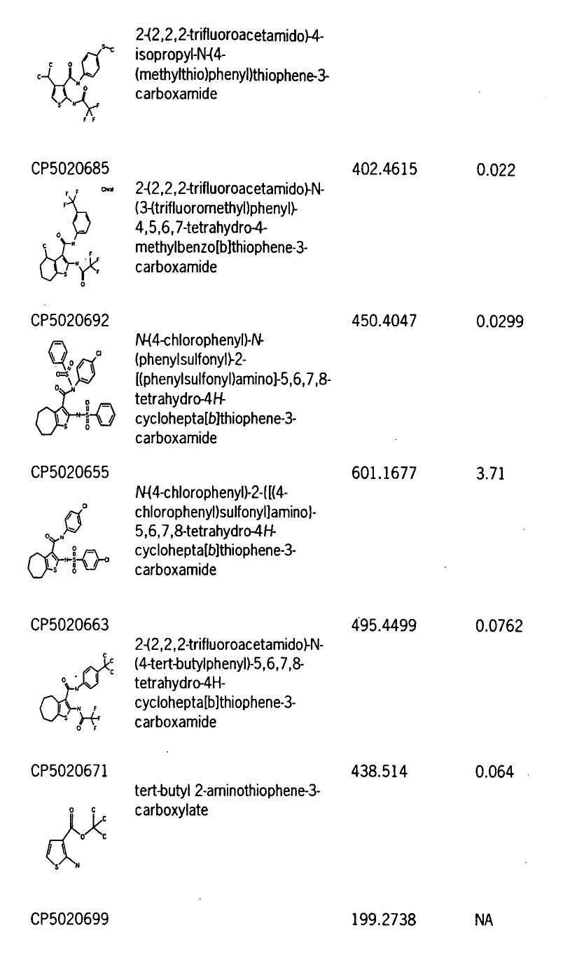

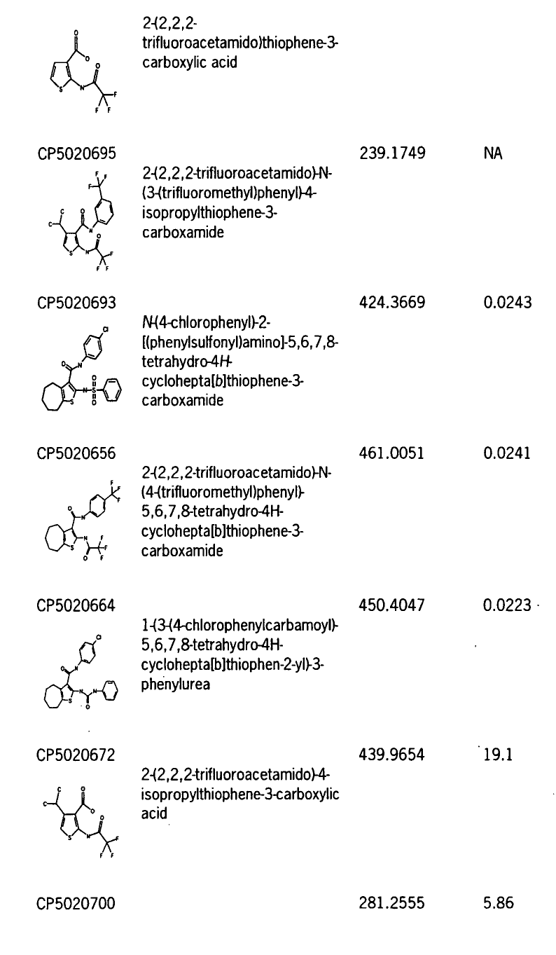

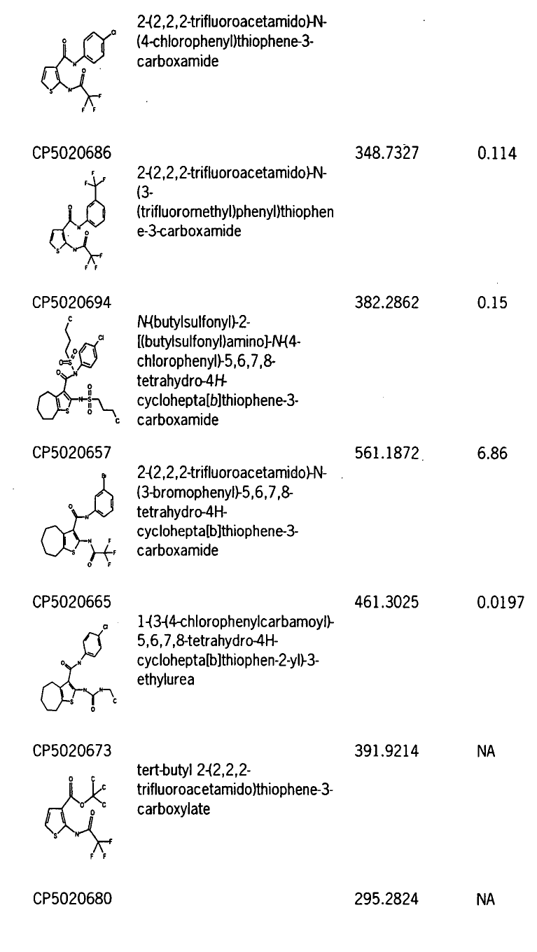

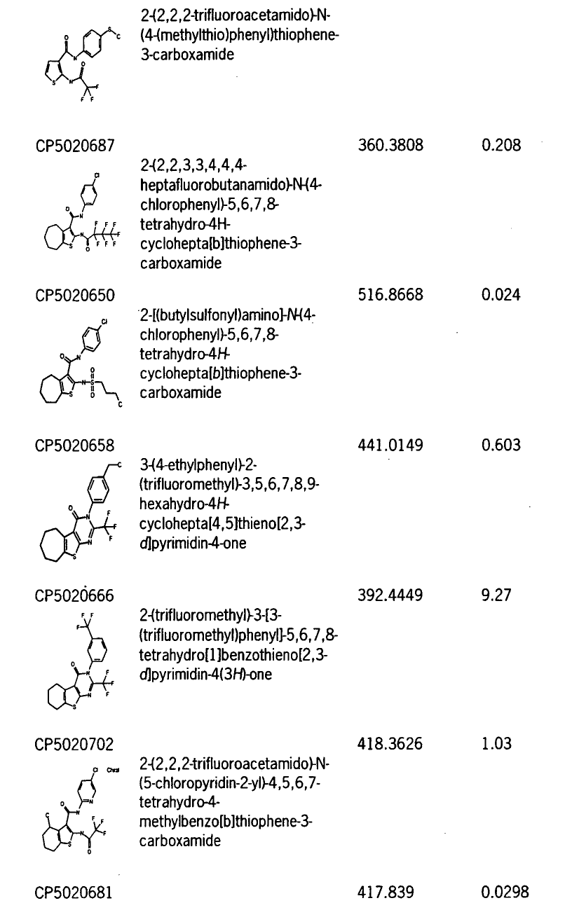

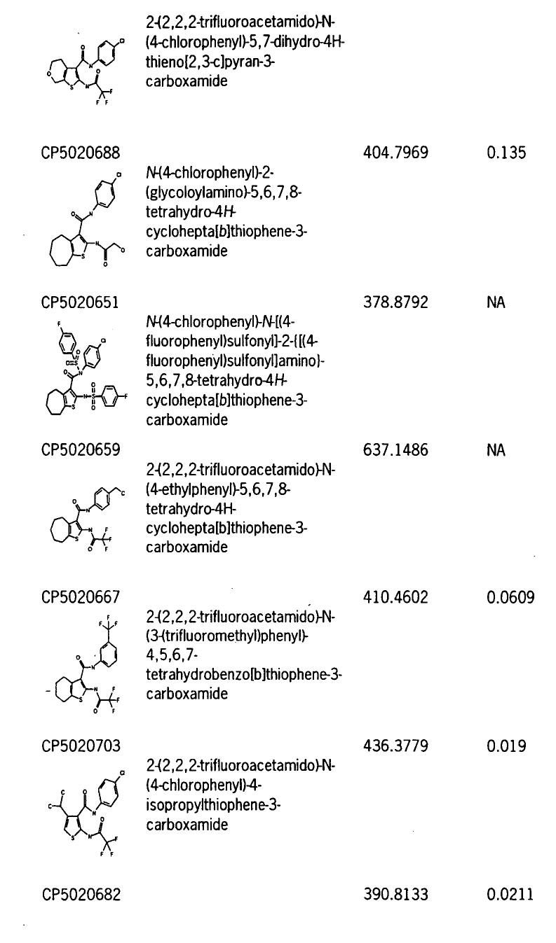

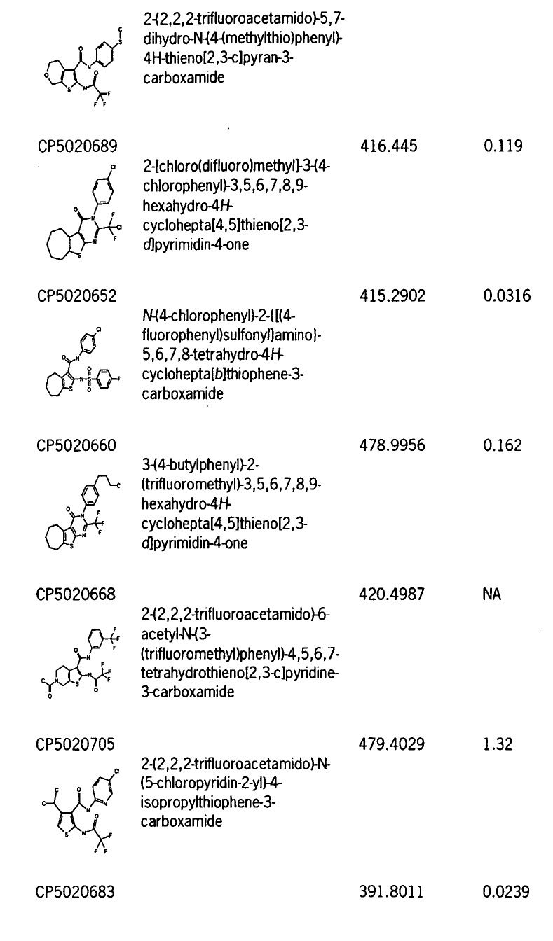

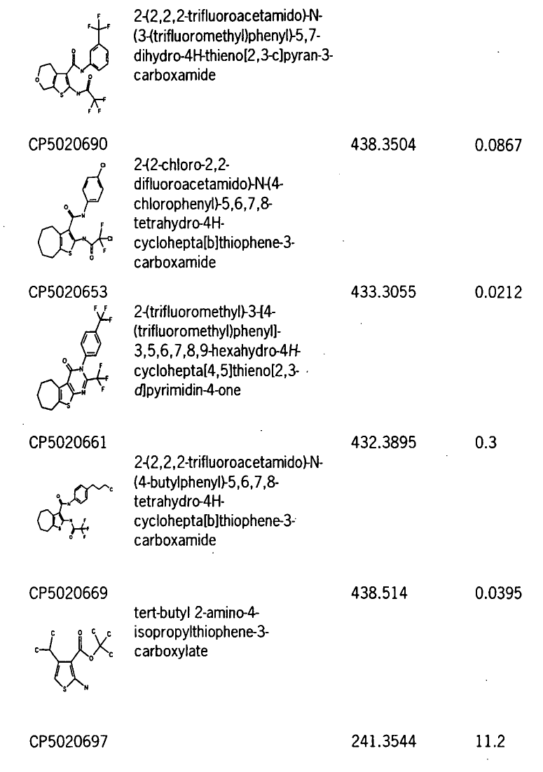

Using the screen disclosed above, a large number of compounds were analyzed for their anti-tumor activity. Compounds according to the present invention with activity against at least one tumor type tested are presented in FIG. 1. IC50 values are reported for the designated tumor type, according to the methods disclosed in the specification. The IC50 values are in micromolar concentrations and the acronyms used in the Tables are as follows:

T=Tumor

NT=Not tested

NT*=The compound showed activity at one, or more concentrations, but an IC50 was not determined; these compounds are considered “active”

NA=No activity observed

These data clearly show that the compounds of the invention can be used as an anti-tumor agent against a variety of tumor types.

1. A compound of the formula: 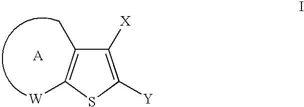
or a pharmaceutically acceptable derivative thereof,
wherein
W is a carbon or nitrogen atom;
Y is —NR¹R² and X is —NR^{5′}R⁶, —C(O)NR⁵R⁶ or —C(O)OR⁶, or
Y is —C(O)NR¹R⁵ and X is —NR^{5′}R⁶, or
Y is NO₂ and X is CH═CH—C(O)OR′;
R¹ and R′ are independently selected from hydrogen or lower alkyl;
R² is selected from hydrogen, —C(O)R¹⁰, —C(O)CH₂OC(O)CH₃, —SO₂R¹⁰;
R⁶ is hydrogen, lower alkyl, —SO₂R^{10′}, or
R² and R⁶, or R² and R⁸, both when Y is —NR¹R², together with respective nitrogen atoms to which they are attached are connected to form a 6-10 membered ring C, which can include a double bond and/or a fused bicyclic ring, wherein Z is —N(R⁵)— or —O—, 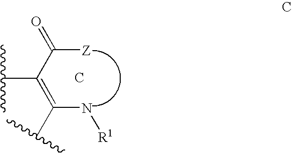
which can be optionally substituted with one or two groups selected from lower alkyl, lower alkenyl, lower alkynyl, trifluoromethyl, difluoromethyl, chlorodifluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷;
R⁵, R^{5′} and R^{5″} are independently hydrogen, or
R⁵, R^{5′} and R^{5″} are independently lower alkyl optionally substituted with one to five groups selected from halo, hydroxyl, lower alkoxy, lower alkenyl and lower alkynyl, or
R⁵, R^{5′} and R^{5″} are independently selected from cycloalkyl, cycloalkylalkyl, heterocyclyl, heterocyclylalkyl, aryl, arylalkyl, heteroaryl or heteroarylalkyl, the ring portion of each is optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷, or
R⁵ and R⁶ together with the nitrogen atom to which they are attached form a 5-7 membered heterocyclic ring optionally substituted with one to three groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷;
R⁷ and R^{7′} are independently selected from hydrogen, lower alkyl, aryl, heteroaryl, arylalkyl, heteroarylalkyl, —C(O)R′ or —C(O)OR′;
R⁸ and R′ are independently selected from hydrogen, lower alkyl and lower alkenyl;
R¹⁰ and R^{10′} are independently selected from —NHR¹⁵, —C(O)OR′, or
R¹⁰ and R^{10′} are independently lower alkyl optionally substituted with one to eight groups selected from halo, hydroxyl, lower alkenyl, lower alkynyl, lower alkoxy, —NR⁸R′, or
R¹⁰ and R^{10′} are independently selected from cycloalkyl, cycloalkylalkyl, heterocyclyl, heterocyclylalkyl, aryl, arylalkyl, heteroaryl or heteroarylalkyl, the ring portion of each is optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷, or
aryl or heteroaryl optionally substituted with one or two groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷;
R¹⁵ is lower alkyl, aryl or heteroaryl; and
the A ring represents a 5-14 membered aryl, heteroaryl, cycloalkyl or heterocycloalkyl ring, each of which is optionally substituted with one to three groups independently selected from lower alkyl, halo, oxo, nitro, —CN, lower alkenyl, lower alkynyl, trifluoromethyl, —C(O)R⁷, C(O)OR′, —C(O)NR^{7′}R⁷ and —NHC(O)R⁷, or
aryl or heteroaryl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷.

2. A compound of claim 1 wherein
the A ring is a 6-10 membered aryl, heteroaryl, cycloalkyl or heterocycloalkyl ring, each of which is optionally substituted with one to three groups independently selected from lower alkyl, halo, oxo, nitro, —CN, lower alkenyl, lower alkynyl, trifluoromethyl, —C(O)R⁷, C(O)OR′, —C(O)NR^{7′}R⁷ and —NHC(O)R⁷,
or aryl or heteroaryl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷.

3. A compound of claim 1 having the formula: 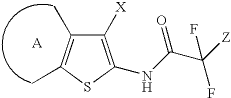
wherein
Z is hydrogen, halo or lower alkyl substituted with from between 2 to 6 halo.

4. A compound of claim 3 wherein Z is hydrogen, chloro, fluoro or —CF₂—CF₂—CF₃.

5. A compound of claim 3 wherein X is —C(O)NR⁵R⁶.

6. A compound of claim 3 wherein
R⁶ is hydrogen; and
R⁵ is aryl or heteroaryl optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷.

7. A compound of claim 6 wherein
R⁵ is phenyl or pyridin-2-yl optionally substituted with one to two groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷.

8. A compound of claim 3 wherein
the A ring is a 6-10 membered aryl, heteroaryl, cycloalkyl or heterocycloalkyl ring, each of which is optionally substituted with one to three groups independently selected from lower alkyl, halo, oxo, nitro, —CN, lower alkenyl, lower alkynyl, trifluoromethyl, —C(O)R⁷, C(O)OR′, —C(O)NR^{7′}R⁷ and —NHC(O)R⁷,
or aryl or heteroaryl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷.

9. A compound according to claim 1 having the formula: 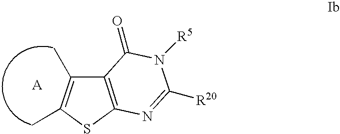
wherein
R²⁰ is selected from lower alkyl, lower alkenyl, lower alkynyl, trifluoromethyl, difluoromethyl, chlorodifluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷.

10. A compound according to claim 9 wherein R²⁰ is trifluoromethyl or chlorodifluoromethyl.

11. A compound according to claim 9 wherein
R⁵ is aryl or heteroaryl optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷.

12. A compound according to claim 11 wherein
R⁵ is phenyl or pyridin-2-yl optionally substituted with one to two groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷.

13. A compound according to claim 9 wherein
the A ring is a 6-10 membered aryl, heteroaryl, cycloalkyl or heterocycloalkyl ring, each of which is optionally substituted with one to three groups independently selected from lower alkyl, halo, oxo, nitro, —CN, lower alkenyl, lower alkynyl, trifluoromethyl, —C(O)R⁷, C(O)OR′, —C(O)NR^{7′}R⁷ and —NHC(O)R⁷; or aryl or heteroaryl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷.

14. A compound according to claim 1 having the formula: 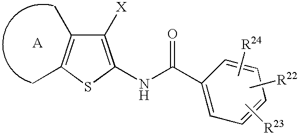
wherein
R²², R²³ and R²⁴ are independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷, or
aryl or heteroaryl optionally substituted with one or two groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷.

15. A compound according to claim 14 wherein X is —C(O)NR⁵R⁶.

16. A compound according to claim 14 wherein
R⁶ is hydrogen; and
R⁵ is aryl or heteroaryl optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷.

17. A compound according to claim 16 wherein
R⁵ is phenyl or pyridin-2-yl optionally substituted with one to two groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷.

18. A compound according to claim 14 wherein
the A ring is a 6-10 membered aryl, heteroaryl, cycloalkyl or heterocycloalkyl ring, each of which is optionally substituted with one to three groups independently selected from lower alkyl, halo, oxo, nitro, —CN, lower alkenyl, lower alkynyl, trifluoromethyl, —C(O)R⁷, C(O)OR′, —C(O)NR^{7′}R⁷ and —NHC(O)R⁷, or
aryl or heteroaryl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷.

19. A compound according to claim 14 wherein
R²² is hydrogen, lower alkyl or lower alkoxy and R²³ is selected from hydrogen, halo, —SR′, lower alkoxy and lower alkyl.

20. A compound according to claim 1 having the formula: 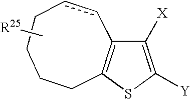
wherein
— is an optional bond; and
R²⁵ is selected from lower alkyl, halo, oxo, nitro, —CN, lower alkenyl, lower alkynyl, trifluoromethyl, —C(O)R⁷, C(O)OR′, —C(O)NR^{7′}R⁷ and —NHC(O)R⁷, or
aryl or heteroaryl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷.

21. A compound according to claim 20 wherein X is —C(O)NR⁵R⁶.

22. A compound according to claim 20 wherein
R⁶ is hydrogen; and
R⁵ is aryl or heteroaryl optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷.

23. A compound according to claim 22 wherein
R⁵ is phenyl or pyridin-2-yl optionally substituted with one to two groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷.

24. A compound according to claim 20 wherein
Y is —NC(O)—R¹⁰; and
R¹⁰ is selected from lower alkyl optionally substituted with one to eight groups selected from halo, hydroxyl, lower alkenyl, lower alkynyl, lower alkoxy, —NR⁸R′, or
R¹⁰ is selected from cycloalkyl, cycloalkylalkyl, heterocyclyl, heterocyclylalkyl, aryl, arylalkyl, heteroaryl or heteroarylalkyl, the ring portion of each is optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷, or
aryl or heteroaryl optionally substituted with one or two groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷.

25. A compound according to claim 24 wherein
R¹⁰ is lower alkyl substituted with 1-3 groups independently selected from halo, or
R¹⁰ is aryl or heteroaryl optionally substituted with lower alkyl or halo.

26. A compound according to claim 20 wherein
X and Y form the c ring.

27. A compound according to claim 20 wherein R²⁵ is hydrogen.

28. A compound according to claim 27 having the formula: 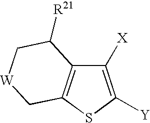
wherein
W is selected from —O—, —S—, —C(R²⁶)(R²⁸)— and —NR³⁰—;
R²¹ is hydrogen or lower alkyl; and
R²⁶, R²⁸ and R³⁰ are independently selected from optionally substituted with one to three groups independently selected from lower alkyl, halo, oxo, nitro, —CN, lower alkenyl, lower alkynyl, trifluoromethyl, —C(O)R⁷, C(O)OR′, —C(O)NR^{7′}R⁷ and —NHC(O)R⁷; or aryl or heteroaryl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷; or R²⁶ and R²⁸ together can form a cycloalkyl or heterocycloalkyl of from between 4-6 members.

29. A compound according to claim 28 wherein X is —C(O)NR⁵R⁶.

30. A compound according to claim 28 wherein
R⁶ is hydrogen; and
R⁵ is aryl or heteroaryl optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷.

31. A compound according to claim 30 wherein
R⁵ is phenyl or pyridin-2-yl optionally substituted with one to two groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷.

32. A compound according to claim 28 wherein
Y is —NC(O)—R¹⁰; and
R¹⁰ is selected from lower alkyl optionally substituted with one to eight groups selected from halo, hydroxyl, lower alkenyl, lower alkynyl, lower alkoxy, —NR⁸R′, or
R¹⁰ is selected from cycloalkyl, cycloalkylalkyl, heterocyclyl, heterocyclylalkyl, aryl, arylalkyl, heteroaryl or heteroarylalkyl, the ring portion of each is optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷, or
aryl or heteroaryl optionally substituted with one or two groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷.

33. A compound according to claim 32
R¹⁰ is lower alkyl substituted with 1-3 groups independently selected from halo, or
R¹⁰ is aryl or heteroaryl optionally substituted with lower alkyl or halo.

34. A compound according to claim 28 wherein X and Y form the c ring.

35. A compound according to claim 28 wherein R²⁶ and R²⁸ are selected from hydrogen and 1,3-dioxolan-2-yl.

36. A compound according to claim 28 wherein R³⁰ is hydrogen or —C(O)OR′.

37. A compound according to claim 1 having the formula: 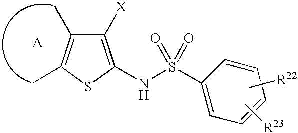
wherein
R²² and R²³ are independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷, or
aryl or heteroaryl optionally substituted with one or two groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷.

38. A compound according to claim 37 wherein X is —C(O)NR⁵R⁶.

39. A compound according to claim 37 wherein
R⁶ is hydrogen; and
R⁵ is aryl or heteroaryl optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷.

40. A compound according to claim 39 wherein
R⁵ is phenyl or pyridin-2-yl optionally substituted with one to two groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷.

41. A compound according to claim 37 wherein
the A ring is a 6-10 membered aryl, heteroaryl, cycloalkyl or heterocycloalkyl ring, each of which is optionally substituted with one to three groups independently selected from lower alkyl, halo, oxo, nitro, —CN, lower alkenyl, lower alkynyl, trifluoromethyl, —C(O)R⁷, C(O)OR′, —C(O)NR^{7′}R⁷ and —NHC(O)R⁷, or
aryl or heteroaryl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷.

42. A compound according to claim 37 wherein R²² is hydrogen and
R²³ is selected from hydrogen, halo and lower alkyl.

43. A compound according to claim 1 having the formula: 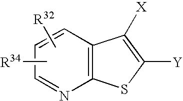
wherein
R³² and R³⁴ are independently selected from lower alkyl, halo, nitro, —CN, lower alkenyl, lower alkynyl, trifluoromethyl, —C(O)R⁷, C(O)OR′, —C(O)NR^{7′}R⁷ and —NHC(O)R⁷; or aryl, heteroaryl, cycloalkyl or heterocycloalkyl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷.

44. A compound according to claim 43 wherein X is —NR^{5′}R⁶.

45. A compound according to claim 44 wherein R^{5′} and R⁶ are both hydrogen.

46. A compound according to claim 43 wherein
Y is —NC(O)—R¹⁰; and
R¹⁰ is selected from lower alkyl optionally substituted with one to eight groups selected from halo, hydroxyl, lower alkenyl, lower alkynyl, lower alkoxy, —NR⁸R′, or
R¹⁰ is selected from cycloalkyl, cycloalkylalkyl, heterocyclyl, heterocyclylalkyl, aryl, arylalkyl, heteroaryl or heteroarylalkyl, the ring portion of each is optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷,
or aryl or heteroaryl optionally substituted with one or two groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷. In a preferred embodiment, R¹⁰ is lower alkyl substituted with 1-3 groups independently selected from halo, or R¹⁰ is aryl or heteroaryl optionally substituted with lower alkyl or halo.

47. A compound according to claim 43 wherein
R³² is hydrogen and R³⁴ is selected from aryl or heteroaryl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷.

48. A compound according to claim 1 having the formula: 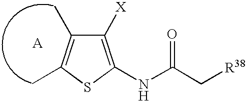
wherein
R³⁸ is selected from aryl or heteroaryl, each of which is optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷, or
aryl or heteroaryl optionally substituted with one or two groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷.

49. A compound according to claim 48 wherein X is —C(O)NR⁵R⁶.

50. A compound according to claim 48 wherein
R³⁸ is aryl or heteroaryl optionally substituted by one or two groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷, or
aryl or heteroaryl optionally substituted with one or two groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷.

51. A compound according to claim 1 having the formula: 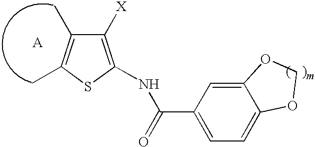
wherein
m is 1 or 2.

52. A compound according to claim 52 wherein X is —NR^{5′}R⁶.

53. A compound according to claim 53 wherein R^{5′} and R⁶ are both hydrogen.

54. A compound according to claim 52 wherein
the A ring is a 6-membered aryl or heteroaryl group optionally substituted by one or two groups selected from lower alkyl, halo, nitro, —CN, lower alkenyl, lower alkynyl, trifluoromethyl, —C(O)R⁷, C(O)OR′, —C(O)NR^{7′}R⁷ and —NHC(O)R⁷, or
aryl, heteroaryl, cycloalkyl or heterocycloalkyl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷.

55. A compound according to claim 1 having the formula: 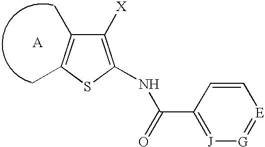
wherein
one of E, G and J is N and the other two are C—R^{22′}; and
R^{22′} is hydrogen, or
R²² is lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷, or
aryl or heteroaryl optionally substituted with one or two groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷.

56. A compound according to claim 55 wherein X is —C(O)NR⁵R⁶.

57. A compound according to claim 55 wherein one of E or G is N and the other two of E, G or J is C—R^{22′}.

58. A compound of the formula: 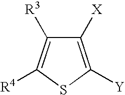
or a pharmaceutically acceptable derivative thereof,
wherein
Y is —NR¹R² and X is —C(O)NR⁵R⁶ or —C(O)OR⁶, or
Y is —C(O)NR¹R⁵ and X is —NR^{5′}R⁶, or
Y is NO₂ and X is CH═CH—C(O)OR′;
R¹ and R′ are independently selected from hydrogen or lower alkyl;
R² is selected from hydrogen, —C(O)R¹⁰, —C(O)CH₂OC(O)CH₃, —SO₂R¹⁰;
R⁶ is hydrogen, lower alkyl, —SO₂R^{10′}, or
R² and R⁶, or R² and R⁸, both when Y is —NR¹R², together with respective nitrogen atoms to which they are attached are connected to form a 6-10 membered ring C, which can include a double bond and/or a fused bicyclic ring, wherein Z is —N(R⁵)— or —O—, 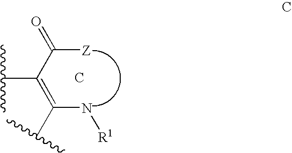
which can be optionally substituted with one or two groups selected from lower alkyl, lower alkenyl, lower alkynyl, trifluoromethyl, difluoromethyl, chlorodifluoromethyl, —C(O)R′, C(O)OR′, halo, amino, lower alkoxy, hydroxyl, monoalkylamino, dialkylamino, nitro, —CN, —SR′, —SO₂R′, —C(O)R⁷, —C(O)NR^{7′}R⁷ and —NHC(O)R⁷;
R³ and R⁴ are independently selected from hydrogen, lower alkyl, lower alkenyl, lower alkynyl, lower alkoxy, halo, —C(O)OR′, —C(O)NHR^{5″}, or
R³ is aryl optionally substituted with lower alkyl, lower alkoxy or halo, or
R³ and R⁴ together with the carbon atoms to which they are attached form a 5-14 membered aryl, heteroaryl, cycloalkyl or heterocycloalkyl ring, each of which is optionally substituted with one to three groups independently selected from lower alkyl, halo, oxo, nitro, —CN, lower alkenyl, lower alkynyl, trifluoromethyl, —C(O)R⁷, C(O)OR′, —C(O)NR^{7′}R⁷ and —NHC(O)R⁷, or
aryl or heteroaryl optionally substituted with one to three groups selected from lower alkyl, halo, oxo, nitro, —CN, lower alkenyl, lower alkynyl, trifluoromethyl, lower alkoxy, hydroxyl, —C(O)R⁷, C(O)OR′, —C(O)NR^{7′}R⁷ and —NHC(O)R⁷;
R⁵, R^{5′} and R^{5″} are independently hydrogen, or
R⁵, R^{5′} and R^{5″} are independently lower alkyl optionally substituted with one to five groups selected from halo, hydroxyl, lower alkoxy, lower alkenyl and lower alkynyl, or
R⁵, R^{5′} and R^{5″} are independently selected from cycloalkyl, cycloalkylalkyl, heterocyclyl, heterocyclylalkyl, aryl, arylalkyl, heteroaryl or heteroarylalkyl, the ring portion of each is optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷, or
R⁵ and R⁶ together with the nitrogen atom to which they are attached form a 5-7 membered heterocyclic ring optionally substituted with one to three groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R⁷, —C(O)NR^{7′}R⁷ and —NHC(O)R⁷;
R⁷ and R^{7′} are independently selected from hydrogen, lower alkyl, aryl, heteroaryl, arylalkyl, heteroarylalkyl, —C(O)R′ or —C(O)OR′;
R⁸ and R′ are independently selected from hydrogen, lower alkyl and lower alkenyl;
R¹⁰ and R^{10′} are independently selected from —NHR¹⁵, —C(O)OR′, or
R¹⁰ and R^{10′} are independently lower alkyl optionally substituted with one to eight groups selected from halo, hydroxyl, lower alkenyl, lower alkynyl, lower alkoxy, —NR⁸R′, or
R¹⁰ and R^{10′} are independently selected from cycloalkyl, cycloalkylalkyl, heterocyclyl, heterocyclylalkyl, aryl, arylalkyl, heteroaryl or heteroarylalkyl, the ring portion of each is optionally substituted with one to four groups independently selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, oxo, —CN, —SR′, —SO₂R′, —C(O)R′, —C(O)NR^{7′}R⁷ and —NHC(O)R⁷, or
aryl or heteroaryl optionally substituted with one or two groups selected from lower alkyl, lower alkenyl, lower alkynyl, trifluoromethyl, difluoromethyl, —C(O)R′, C(O)OR′, halo, amino, lower alkoxy, hydroxyl, monoalkylamino, dialkylamino, nitro, —CN, —SR′, —SO²R′, —C(O)R⁷, —C(O)NR^{7′}R⁷ and —NHC(O)R⁷; and
R¹⁵ is lower alkyl, aryl or heteroaryl.

59. A compound according to claim 58 having the formula: 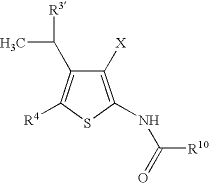
wherein
R^{3′} is hydrogen or lower alkyl.

60. A compound according to claim 58 having the formula: 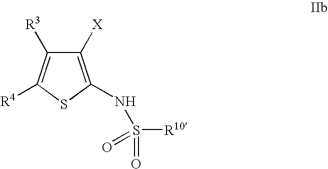
wherein X, R³, R⁴ and R^{10′} is lower alkyl.

61. A compound according to claim 1 and selected from:
2-(2,2,2-trifluoroacetamido)-4,5,6,7,8,9,10,11,12,13-decahydrocyclododeca[b]thiophene-3-carboxamide;
N-(3-carbamoyl-4,5-dimethylthiophen-2-yl)-2-(3,4-dimethoxyphenyl)quinoline-4-carboxamide;
N-(3-carbamoyl-5-methyl-4-phenylthiophen-2-yl)-8-methoxy-2-oxo-2H-chromene-3-carboxamide;
5-(2-(5-methylfuran-2-yl)quinoline-4-carboxamido)-N2,N2-diethyl-3-methylthiophene-2,4-dicarboxamide;
2-(2-(4-tert-butylphenyl)cyclopropanecarboxamido)-4-(4-fluorophenyl)-5-methylthiophene-3-carboxamide;
N-(3-carbamoyl-4-(4-ethylphenyl)-5-methylthiophen-2-yl)-2-(5-methylfuran-2-yl)quinoline-4-carboxamide;
ethyl 5-(4-bromo-1-ethyl-1H-pyrazole-5-carboxamido)-4-carbamoyl-3-methylthiophene-2-carboxylate;
N-(3-carbamoyl-5-methyl-4-(3,4-dimethylphenyl)thiophen-2-yl)-2-(5-methylfuran-2-yl)quinoline-4-carboxamide;
2-({3-[hydroxy(oxido)amino]-4-methoxybenzoyl}amino)-5-methyl-4-phenylthiophene-3-carboxamide;
methyl 5-(2-(5-methylfuran-2-yl)quinoline-4-carboxamido)-4-carbamoyl-3-methylthiophene-2-carboxylate;
N-[3-(aminocarbonyl)-4-(4-fluorophenyl)-5-methyl-2-thienyl]-2-(5-methyl-2-furyl)quinoline-4-carboxamide;
N-(3-carbamoyl-5-methyl-4-phenylthiophen-2-yl)-2-(5-methylfuran-2-yl)quinoline-4-carboxamide;
N-(3-carbamoyl-5,6-dihydro-4H-cyclopenta[b]thiophen-2-yl)-7-(difluoromethyl)-5-phenylpyrazolo[1,5-a]pyrimidine-3-carboxamide;
2-(2-ethoxybenzamido)-4,5-dimethylthiophene-3-carboxamide methyl 4-(2-(3-fluorobenzamido)-4,5,6,7-tetrahydrobenzo[b]thiophene-3-carboxamido)benzoate;
2-(2,2,2-trifluoroacetamido)-N-(4-chlorophenyl)-4-isopropylthiophene-3-carboxamide;
2-(2,2,2-trifluoroacetamido)-4-isopropyl-N-(4-(methylthio)phenyl)thiophene-3-carboxamide;
2-(2,2,2-trifluoroacetamido)-N-(5-chloropyridin-2-yl)-4-isopropylthiophene-3-carboxamide;
2-(2,2,2-trifluoroacetamido)-N-(3-(trifluoromethyl)phenyl)-4-isopropylthiophene-3-carboxamide;
2-(2,2,2-trifluoroacetamido)-N-(4-chlorophenyl)thiophene-3-carboxamide;
2-(2,2,2-trifluoroacetamido)-N-(3-(trifluoromethyl)phenyl)thiophene-3-carboxamide;
2-(2,2,2-trifluoroacetamido)-N-(4-(methylthio)phenyl)thiophene-3-carboxamide;
2,5-bis(3,4-dichlorophenyl)-4-hydroxythiophen-3(2H)-one 1,1-dioxide;
2-(2,2,2-trifluoroacetamido)-4-isopropylthiophene-3-carboxylic acid;
N-(2-(2-(2-(2-(5-(3,4-dichlorophenylamino)-[1,2,5]oxadiazolo[3,4-b]pyrazin-6-ylamino)ethoxy)ethoxy)ethoxy)ethyl)-5-(hexahydro-2-oxo-1H-thieno[3,4-d]imidazol-6-yl)pentanamide;
tert-butyl 2-(2,2,2-trifluoroacetamido)-4-isopropylthiophene-3-carboxylate
tert-butyl 2-amino-4-isopropylthiophene-3-carboxylate;
2-(trifluoromethyl)-5-isopropyl-3-(4-(methylthio)phenyl)thieno[2,3-d]pyrimidin-4(3H)-one;
2-(2,2,2-trifluoroacetamido)-N-(3-(trifluoromethyl)phenyl)-4,5,6,7,8,9-hexahydrocycloocta[b]thiophene-3-carboxamide;
2-(2,2-difluoroacetamido)-N-(4-chlorophenyl)-5,6,7,8-tetrahydro-4H-cyclohepta[b]thiophene-3-carboxamide;
N-(4-chlorophenyl)-N-[(4-chlorophenyl)sulfonyl]-2-{[(4-chlorophenyl)sulfonyl]amino}-5,6,7,8-tetrahydro-4H-cyclohepta[b]thiophene-3-carboxamide;
3-(4-tert-butylphenyl)-2-(trifluoromethyl)-3,5,6,7,8,9-hexahydro-4H-cyclohepta[4,5]thieno[2,3-d]pyrimidin-4-one;
tert-butyl 2-(2,2,2-trifluoroacetamido)-4-isopropylthiophene-3-carboxylate 2-(2,2,2-trifluoroacetamido)-4-isopropyl-N-(4-(methylthio)phenyl)thiophene-3-carboxamide;
2-(2,2,2-trifluoroacetamido)-N-(3-(trifluoromethyl)phenyl)-4,5,6,7-tetrahydro-4-methylbenzo[b]thiophene-3-carboxamide;
N-(4-chlorophenyl)-N-(phenylsulfonyl)-2-[(phenylsulfonyl)amino]-5,6,7,8-tetrahydro-4H-cyclohepta[b]thiophene-3-carboxamide;
N-(4-chlorophenyl)-2-{[(4-chlorophenyl)sulfonyl]amino}-5,6,7,8-tetrahydro-4H-cyclohepta[b]thiophene-3-carboxamide;
2-(2,2,2-trifluoroacetamido)-N-(4-tert-butylphenyl)-5,6,7,8-tetrahydro-4H-cyclohepta[b]thiophene-3-carboxamide;
tert-butyl 2-aminothiophene-3-carboxylate;
2-(2,2,2-trifluoroacetamido)thiophene-3-carboxylic acid;
2-(2,2,2-trifluoroacetamido)-N-(3-(trifluoromethyl)phenyl)-4-isopropylthiophene-3-carboxamide;
N-(4-chlorophenyl)-2-[(phenylsulfonyl)amino]-5,6,7,8-tetrahydro-4H-cyclohepta[b]thiophene-3-carboxamide;
2-(2,2,2-trifluoroacetamido)-N-(4-(trifluoromethyl)phenyl)-5,6,7,8-tetrahydro-4H-cyclohepta[b]thiophene-3-carboxamide;
1-(3-(4-chlorophenylcarbamoyl)-5,6,7,8-tetrahydro-4H-cyclohepta[b]thiophen-2-yl)-3-phenylurea;
2-(2,2,2-trifluoroacetamido)-4-isopropylthiophene-3-carboxylic acid;
2-(2,2,2-trifluoroacetamido)-N-(4-chlorophenyl)thiophene-3-carboxamide;
2-(2,2,2-trifluoroacetamido)-N-(3-(trifluoromethyl)phenyl)thiophene-3-carboxamide;
N-(butylsulfonyl)-2-[(butylsulfonyl)amino]-N-(4-chlorophenyl)-5,6,7,8-tetrahydro-4H-cyclohepta[b]thiophene-3-carboxamide;
2-(2,2,2-trifluoroacetamido)-N-(3-bromophenyl)-5,6,7,8-tetrahydro-4H-cyclohepta[b]thiophene-3-carboxamide;
1-(3-(4-chlorophenylcarbamoyl)-5,6,7,8-tetrahydro-4H-cyclohepta[b]thiophen-2-yl)-3-ethylurea;
tert-butyl 2-(2,2,2-trifluoroacetamido)thiophene-3-carboxylate;
2-(2,2,2-trifluoroacetamido)-N-(4-(methylthio)phenyl)thiophene-3-carboxamide;
2-(2,2,3,3,4,4,4-heptafluorobutanamido)-N-(4-chlorophenyl)-5,6,7,8-tetrahydro-4H-cyclohepta[b]thiophene-3-carboxamide;
2-[(butylsulfonyl)amino]-N-(4-chlorophenyl)-5,6,7,8-tetrahydro-4H-cyclohepta[b]thiophene-3-carboxamide;
3-(4-ethylphenyl)-2-(trifluoromethyl)-3,5,6,7,8,9-hexahydro-4H-cyclohepta[4,5]thieno[2,3-d]pyrimidin-4-one;
2-(trifluoromethyl)-3-[3-(trifluoromethyl)phenyl]-5,6,7,8-tetrahydro[1]benzothieno[2,3-d]pyrimidin-4(3H)-one;
2-(2,2,2-trifluoroacetamido)-N-(5-chloropyridin-2-yl)-4,5,6,7-tetrahydro-4-methylbenzo[b]thiophene-3-carboxamide;
2-(2,2,2-trifluoroacetamido)-N-(4-chlorophenyl)-5,7-dihydro-4H-thieno[2,3-c]pyran-3-carboxamide;
N-(4-chlorophenyl)-2-(glycoloylamino)-5,6,7,8-tetrahydro-4H-cyclohepta[b]thiophene-3-carboxamide;
N-(4-chlorophenyl)-N-[(4-fluorophenyl)sulfonyl]-2-{[(4-fluorophenyl)sulfonyl]amino}-5,6,7,8-tetrahydro-4H-cyclohepta[b]thiophene-3-carboxamide;
2-(2,2,2-trifluoroacetamido)-N-(4-ethylphenyl)-5,6,7,8-tetrahydro-4H-cyclohepta[b]thiophene-3-carboxamide;
2-(2,2,2-trifluoroacetamido)-N-(3-(trifluoromethyl)phenyl)-4,5,6,7-tetrahydrobenzo[b]thiophene-3-carboxamide;
2-(2,2,2-trifluoroacetamido)-N-(4-chlorophenyl)-4-isopropylthiophene-3-carboxamide;
2-(2,2,2-trifluoroacetamido)-5,7-dihydro-N-(4-(methylthio)phenyl)-4H-thieno[2,3-c]pyran-3-carboxamide;
2-[chloro(difluoro)methyl]-3-(4-chlorophenyl)-3,5,6,7,8,9-hexahydro-4H-cyclohepta[4,5]thieno[2,3-d]pyrimidin-4-one;
N-(4-chlorophenyl)-2-{[(4-fluorophenyl)sulfonyl]amino}-5,6,7,8-tetrahydro-4H-cyclohepta[b]thiophene-3-carboxamide;
3-(4-butylphenyl)-2-(trifluoromethyl)-3,5,6,7,8,9-hexahydro-4H-cyclohepta[4,5]thieno[2,3-d]pyrimidin-4-one;
2-(2,2,2-trifluoroacetamido)-6-acetyl-N-(3-(trifluoromethyl)phenyl)-4,5,6,7-tetrahydrothieno[2,3-c]pyridine-3-carboxamide;
2,2,2-trifluoroacetamido)-N-(5-chloropyridin-2-yl)-4-isopropylthiophene-3-carboxamide;
2-(2,2,2-trifluoroacetamido)-N-(3-(trifluoromethyl)phenyl)-5,7-dihydro-4H-thieno[2,3-c]pyran-3-carboxamide;
2-(2-chloro-2,2-difluoroacetamido)-N-(4-chlorophenyl)-5,6,7,8-tetrahydro-4H-cyclohepta[b]thiophene-3-carboxamide;
2-(trifluoromethyl)-3-[4-(trifluoromethyl)phenyl]-3,5,6,7,8,9-hexahydro-4H-cyclohepta[4,5]thieno[2,3-d]pyrimidin-4-one;
2-(2,2,2-trifluoroacetamido)-N-(4-butylphenyl)-5,6,7,8-tetrahydro-4H-cyclohepta[b]thiophene-3-carboxamide;
tert-butyl 2-amino-4-isopropylthiophene-3-carboxylate;
2-(2,2,2-trifluoroacetamido)-N-(3-chlorophenyl)-5,6,7,8-tetrahydro-4H-cyclohepta[b]thiophene-3-carboxamide;
3-(4-methylpyridin-2-yl)-2-(trifluoromethyl)-3,5,6,7,8,9-hexahydro-4H-cyclohepta[4,5]thieno[2,3-d]pyrimidin-4-one;
tert-butyl 2-(2,2,2-trifluoroacetamido)-4,5,6,7,8,9-hexahydrocycloocta[b]thiophene-3-carboxylate;
N-[3-(4-chlorobenzoyl)-5,6,7,8-tetrahydro-4H-cyclohepta[b]thien-2-yl]-2,2,2-trifluoroacetamide;
3-[4-(methylthio)phenyl]-2-(trifluoromethyl)-5,6,7,8-tetrahydro[1]benzothieno[2,3-d]pyrimidin-4(3H)-one;
3-phenyl-2-(trifluoromethyl)-3,5,6,7,8,9-hexahydro-4H-cyclohepta[4,5]thieno[2,3-d]pyrimidin-4-one;
2,2,2-trifluoro-N-[3-(morpholin-4-ylcarbonyl)-5,6,7,8-tetrahydro-4H-cyclohepta[b]thien-2-yl]acetamide;
2-(2,2,2-trifluoroacetamido)-5,7-dihydro-4H-thieno[2,3-c]pyran-3-carboxylic acid;
(2-amino-5,6,7,8-tetrahydro-4H-cyclohepta[b]thiophen-3-yl)(3-(trifluoromethyl)phenyl)methanone;
3-(4-chlorophenyl)-2-(trifluoromethyl)-3,5,6,7,8,9-hexahydro-4H-cyclohepta[4,5]thieno[2,3-d]pyrimidin-4-one;
3-pyridin-2-yl-2-(trifluoromethyl)-3,5,6,7,8,9-hexahydro-4H-cyclohepta[4,5]thieno[2,3-d]pyrimidin-4-one;
tert-butyl 2-[(trifluoroacetyl)amino]-4,5,7,7a-tetrahydro-3aH-spiro[1-benzothiophene-6,2′-[1,3]dioxolane]-3-carboxylate;
N-[3-(3-chlorobenzoyl)-5,6,7,8-tetrahydro-4H-cyclohepta[b]thien-2-yl]-2,2,2-trifluoroacetamide;
3-(3,4-dichlorophenyl)-2-(trifluoromethyl)-3,5,6,7,8,9-hexahydro-4H-cyclohepta[4,5]thieno[2,3-d]pyrimidin-4-one;
3-pyridin-3-yl-2-(trifluoromethyl)-3,5,6,7,8,9-hexahydro-4H-cyclohepta[4,5]thieno[2,3-d]pyrimidin-4-one;
2-(2,2,2-trifluoroacetamido)-4,5,6,7,8,9-hexahydrocycloocta[b]thiophene-3-carboxylic acid;
2,2,2-trifluoro-N-{3-[3-(trifluoromethyl)benzoyl]-5,6,7,8-tetrahydro-4H-cyclohepta[b]thien-2-yl}acetamide;
3-(3,5-dichlorophenyl)-2-(trifluoromethyl)-3,5,6,7,8,9-hexahydro-4H-cyclohepta[4,5]thieno[2,3-d]pyrimidin-4-one;
3-(3-chlorophenyl)-2-(trifluoromethyl)-3,5,6,8-tetrahydro-4H-pyrano[4′,3′:4,5]thieno[2,3-d]pyrimidin-4-one;
(2-amino-5,6,7,8-tetrahydro-4H-cyclohepta[b]thiophen-3-yl)(4-chlorophenyl)methanone;
2-(2,2,2-trifluoroacetamido)-N-(3-chlorophenyl)-5,6,7,8-tetrahydro-4H-cyclohepta[b]thiophene-3-carboxamide;
3-(5-chloropyridin-2-yl)-2-(trifluoromethyl)-3,5,6,7,8,9-hexahydro-4H-cyclohepta[4,5]thieno[2,3-d]pyrimidin-4-one;
3-pyridin-3-yl-2-(trifluoromethyl)-3,5,6,8-tetrahydro-4H-pyrano[4′,3′:4,5]thieno[2,3-d]pyrimidin-4-one;
2-(2,2,2-trifluoroacetamido)-4,5,6,7-tetrahydro-4-methylbenzo[b]thiophene-3-carboxylic acid;
3-(5-chloropyridin-2-yl)-2-(trifluoromethyl)-3,5,6,7,8,9-hexahydro-4H-cyclohepta[4,5]thieno[2,3-d]pyrimidin-4-one;
3-(5-methylpyridin-2-yl)-2-(trifluoromethyl)-3,5,6,7,8,9-hexahydro-4H-cyclohepta[4,5]thieno[2,3-d]pyrimidin-4-one;
3-pyridin-4-yl-2-(trifluoromethyl)-3,5,6,8-tetrahydro-4H-pyrano[4′,3′:4,5]thieno[2,3-d]pyrimidin-4-one;
(2-amino-5,6,7,8-tetrahydro-4H-cyclohepta[b]thiophen-3-yl)(3-chlorophenyl)methanone;
3-(3,4-dimethylphenyl)-2-(trifluoromethyl)-3,5,6,7,8,9-hexahydro-4H-cyclohepta[4,5]thieno[2,3-d]pyrimidin-4-one;
3-pyridin-4-yl-2-(trifluoromethyl)-3,5,6,7,8,9-hexahydro-4H-cyclohepta[4,5]thieno[2,3-d]pyrimidin-4-one;
tert-butyl 2-(2,2,2-trifluoroacetamido)-4,5,6,7-tetrahydro-4-methylbenzo[b]thiophene-3-carboxylate;
3-(4-chlorophenyl)-2-(trifluoromethyl)-3,5,6,8-tetrahydro-4H-spiro[1-benzothieno[2,3-d]pyrimidine-7,2′-[1,3]dioxolan]-4-one;
(Z)-2-(2,2,2-trifluoroacetamido)-N-(3-chlorophenyl)-7,8-dihydro-6H-cyclohepta[b]thiophene-3-carboxamide;
3-amino-N-(4-bromo-2-methylphenyl)-6-(3,4-dimethoxyphenyl)thieno[2,3-b]pyridine-2-carboxamide;
3-amino-N-(2-(butylthio)phenyl)-6-(thiophen-2-yl)thieno[2,3-b]pyridine-2-carboxamide;
N-(4-chlorophenyl)-2-[(trifluoroacetyl)amino]-4,7-dihydro-5H-spiro[1-benzothiophene-6,2′-[1,3]dioxolane]-3-carboxamide;
2-(2,2,2-trifluoroacetamido)-N-(2,2,2-trifluoroethyl)-5,6,7,8-tetrahydro-4H-cyclohepta[b]thiophene-3-carboxamide;
3-amino-N-(2,3-dihydrobenzo[b][1,4]dioxin-7-yl)-6-(3-methoxyphenyl)thieno[2,3-b]pyridine-2-carboxamide;
3-(4-chlorophenyl)-2-(trifluoromethyl)-5,6,7,8,9,10-hexahydrocycloocta[4,5]thieno[2,3-d]pyrimidin-4(3H)-one;
3-(4-chlorophenyl)-2-(trifluoromethyl)-5,6,7,8-tetrahydro[1]benzothieno[2,3-d]pyrimidin-4(3H)-one;
2-(2-(3-(trifluoromethyl)-5-methyl-1H-pyrazol-1-yl)acetamido)-6-tert-butyl-4,5,6,7-tetrahydrobenzo[b]thiophene-3-carboxamide;
3-amino-N-(3-ethoxyphenyl)-6-(3,4-dimethoxyphenyl)thieno[2,3-b]pyridine-2-carboxamide;
3-amino-N-(benzo[d][1,3]dioxol-6-yl)-6-(3-methoxyphenyl)thieno[2,3-b]pyridine-2-carboxamide;
2-(2,2,2-trifluoroacetamido)-N-(4-chlorophenyl)-4,5,6,7,8,9-hexahydrocycloocta[b]thiophene-3-carboxamide;
2-(2,2,2-trifluoroacetamido)-N-(4-chlorophenyl)-4,5,6,7-tetrahydrobenzo[b]thiophene-3-carboxamide;
2-(2-(4-bromo-3-(trifluoromethyl)-5-methyl-1H-pyrazol-1-yl)acetamido)-5,6,7,8-tetrahydro-4H-cyclohepta[b]thiophene-3-carboxamide;
3-amino-N-(3,4,5-trimethoxyphenyl)-6-(thiophen-2-yl)thieno[2,3-b]pyridine-2-carboxamide;
3-amino-6-(3-methoxyphenyl)-N-(3-(methylthio)phenyl)thieno[2,3-b]pyridine-2-carboxamide;
3-(4-chlorophenyl)-5-methyl-2-(trifluoromethyl)-5,6,7,8-tetrahydro[1]benzothieno[2,3-d]pyrimidin-4(3H)-one;
2-(2,2,2-trifluoroacetamido)-N-(4-chlorophenyl)-5,6,7,8-tetrahydro-4H-cyclohepta[b]thiophene-3-carboxamide;
3-amino-6-(3,4-dimethoxyphenyl)-N-(3,4-dimethylphenyl)thieno[2,3-b]pyridine-2-carboxamide;
3-amino-N-(benzo[d][1,3]dioxol-6-yl)-6-(3,4-dimethoxyphenyl)thieno[2,3-b]pyridine-2-carboxamide;
3-amino-6-(3-methoxyphenyl)-N-(3,4-dimethylphenyl)thieno[2,3-b]pyridine-2-carboxamide;
2-(2,2,2-trifluoroacetamido)-N-(4-chlorophenyl)-4,5,6,7-tetrahydro-4-methylbenzo[b]thiophene-3-carboxamide;
3-amino-N-(4-bromo-3-methylphenyl)-6-(3,4-dimethoxyphenyl)thieno[2,3-b]pyridine-2-carboxamide;
3-amino-6-(4-fluorophenyl)-N-(3,4,5-trimethoxyphenyl)thieno[2,3-b]pyridine-2-carboxamide;
3-amino-N-(2-(ethylthio)phenyl)-6-(thiophen-2-yl)thieno[2,3-b]pyridine-2-carboxamide;
2-(2-methoxybenzamido)-4,5,6,7,8,9-hexahydrocycloocta[b]thiophene-3-carboxamide;
3-(3-chlorophenyl)-2-(trifluoromethyl)-3,5,6,8-tetrahydro-4H-pyrano[4′,3′:4,5]thieno[2,3-d]pyrimidin-4-one;
3-amino-N-(4-bromophenyl)-6-(3,4-dimethoxyphenyl)thieno[2,3-b]pyridine-2-carboxamide;
3-amino-N-(4-fluorophenyl)-6-(3,4-dimethoxyphenyl)thieno[2,3-b]pyridine-2-carboxamide;
3-amino-N-(2-(propylthio)phenyl)-6-(thiophen-2-yl)thieno[2,3-b]pyridine-2-carboxamide;
2-(2,2,2-trifluoroacetamido)-N-(3-chlorophenyl)-5,7-dihydro-4H-thieno[2,3-c]pyran-3-carboxamide;
3-amino-N-(3-chloro-4-methylphenyl)-6-(3,4-dimethoxyphenyl)thieno[2,3-b]pyridine-2-carboxamide;
3-amino-N-(2-bromophenyl)-6-(thiophen-2-yl)thieno[2,3-b]pyridine-2-carboxamide
2-(2,2,2-trifluoroacetamido)-N-(3-chlorophenyl)-5,6,7,8-tetrahydro-4H-cyclohepta[b]thiophene-3-carboxamide;
3-(3-chlorophenyl)-2-(trifluoromethyl)-5,6,7,8,9,10-hexahydrocycloocta[4,5]thieno[2,3-d]pyrimidin-4(3H)-one;
3-(3-chlorophenyl)-2-(trifluoromethyl)-3,5,6,8-tetrahydro-4H-spiro[1-benzothieno[2,3-d]pyrimidine-7,2′-[1,3]dioxolan]-4-one;
N-(3,4-difluorophenyl)-4,5,6,7-tetrahydrobenzo[b]thiophene-3-carboxamide;
ethyl 2-(2,2,2-trifluoroacetamido)-4-(4-chlorophenyl)-5-methylthiophene-3-carboxylate;
5,6,7,8-tetrahydro-2-(methyl carbamoylformyl)-4H-cyclohepta[b]thiophene-3-carboxylic acid;
3-[4-(methylthio)phenyl]-2-(trifluoromethyl)-3,5,6,7,8,9-hexahydro-4H-cyclohepta[4,5]thieno[2,3-d]pyrimidin-4-one;
3-(3-methoxyphenyl)-2-(trifluoromethyl)-3,5,6,7,8,9-hexahydro-4H-cyclohepta[4,5]thieno[2,3-d]pyrimidin-4-one;
3-(3-chlorophenyl)-2-(trifluoromethyl)-3,5,6,8-tetrahydro-4H-thiopyrano[4′,3′:4,5]thieno[2,3-d]pyrimidin-4-one;
3-(3-chlorophenyl)-2-(trifluoromethyl)-5,6,7,8-tetrahydropyrido[4′,3′:4,5]thieno[2,3-d]pyrimidin-4(3H)-one;
3-[4-(methylthio)phenyl]-2-(trifluoromethyl)-5,6,7,8,9,10-hexahydrocycloocta[4,5]thieno[2,3-d]pyrimidin-4(3H)-one;
2-(2,2,2-trifluoroacetamido)-4,5,6,7-tetrahydro-6-oxobenzo[b]thiophene-3-carboxylic acid;
N-(3-fluoro-4-methylphenyl)-5-methyl-4-phenylthiophene-3-carboxamide;
tert-butyl 2-(2,2,2-trifluoroacetamido)-4,5,6,7-tetrahydrobenzo[b]thiophene-3-carboxylate;
3-[4-(methylthio)phenyl]-2-(trifluoromethyl)-5,6,7,8-tetrahydro[1]benzothieno[2,3-d]pyrimidin-4(3H)-one;
2-(2,2,2-trifluoroacetamido)-4,5,6,7-tetrahydro-N-(4-(methylthio)phenyl)benzo[b]thiophene-3-carboxamide;
ethyl 3-(3-chlorophenyl)-4-oxo-3,5,6,7,8,9-hexahydro-4H-cyclohepta[4,5]thieno[2,3-d]pyrimidine-2-carboxylate;
tert-butyl 6-acetyl-2-amino-4,5,6,7-tetrahydrothieno[2,3-c]pyridine-3-carboxylate;
3-(3-chlorophenyl)-2-(trifluoromethyl)-3,5,6,8-tetrahydro-4H-thiopyrano[4′,3′:4,5]thieno[2,3-d]pyrimidin-4-one 7-oxide;
3-(3-chlorophenyl)-5-methyl-2-(trifluoromethyl)-5,6,7,8-tetrahydro[1]benzothieno[2,3-d]pyrimidin-4(3H)-one;
3-(4-chlorobenzyl)-2-(trifluoromethyl)-3,5,6,7,8,9-hexahydro-4H-cyclohepta[4,5]thieno[2,3-d]pyrimidin-4-one;
6-tert-butyl-4,5,6,7-tetrahydro-N-(3-(methylthio)phenyl)benzo[b]thiophene-3-carboxamide;
3-(4-methylphenyl)-2-(trifluoromethyl)-5,6,7,8-tetrahydro[1]benzothieno[2,3-d]pyrimidin-4(3H)-one;
3-[4-(methylsulfonyl)phenyl]-2-(trifluoromethyl)-5,6,7,8-tetrahydro[1]benzothieno[2,3-d]pyrimidin-4(3H)-one;
ethyl 3-(3-chlorophenyl)-4-oxo-3,4,5,6,7,8-hexahydro[1]benzothieno[2,3-d]pyrimidine-2-carboxylate;
tert-butyl 2-amino-5,7-dihydro-4H-thieno[2,3-c]thiopyran-3-carboxylate;
3-tert-butyl 6-propyl 2-[(trifluoroacetyl)amino]-4,7-dihydrothieno[2,3-c]pyridine-3,6(5H)-dicarboxylate;
3-(3-chlorophenyl)-2-(trifluoromethyl)-3,5,6,8-tetrahydro-4H-thiopyrano[4′,3′:4,5]thieno[2,3-d]pyrimidin-4-one 7,7-dioxide;
5-methyl-3-[4-(methylthio)phenyl]-2-(trifluoromethyl)-5,6,7,8-tetrahydro[1]benzothieno[2,3-d]pyrimidin-4(3H)-one;
3-(4-chlorophenyl)-2-(trifluoromethyl)-3,5,6,8-tetrahydro[1]benzothieno[2,3-d]pyrimidine-4,7-dione;
dimethyl 2-(2,2,2-trifluoroacetamido)-5,6-dihydro-4H-cyclopenta[b]thiophene-3,4-dicarboxylate;
3-(3-chlorophenyl)-2-(trifluoromethyl)-5,6,7,8-tetrahydro[1]benzothieno[2,3-d]pyrimidin-4(3H)-one;
3-[4-(methylsulfinyl)phenyl]-2-(trifluoromethyl)-5,6,7,8-tetrahydro[1]benzothieno[2,3-d]pyrimidin-4(3H)-one;
2-(trifluoromethyl)-3-[3-(trifluoromethyl)phenyl]-3,5,6,7,8,9-hexahydro-4H-cyclohepta[4,5]thieno[2,3-d]pyrimidin-4-one;
3-(3-chlorophenyl)-3,5,6,7,8,9-hexahydro-4H-cyclohepta[4,5]thieno[2,3-d]pyrimidin-4-one;
tert-butyl 2-(2,2,2-trifluoroacetamido)-6-acetyl-4,5,6,7-tetrahydrothieno[2,3-c]pyridine-3-carboxylate;
ethyl 4-oxo-3-(2,2,2-trifluoroethyl)-3,5,6,7,8,9-hexahydro-4H-cyclohepta[4,5]thieno[2,3-d]pyrimidine-2-carboxylate;
2-(2,2,2-trifluoroacetamido)-N-(2-chlorophenyl)-5,6,7,8-tetrahydro-4H-cyclohepta[b]thiophene-3-carboxamide;
3-(5-chloropyridin-2-yl)-2-(trifluoromethyl)-5,6,7,8,9,10-hexahydrocycloocta[4,5]thieno[2,3-d]pyrimidin-4(3H)-one;
2-ethyl 4-methyl 5-(2,2,2-trifluoroacetamido)-3-methylthiophene-2,4-dicarboxylate;
3-(5-chloropyridin-2-yl)-2-(trifluoromethyl)-5,6,7,8-tetrahydro[1]benzothieno[2,3-d]pyrimidin-4(3H)-one;
3-[3-(methylthio)phenyl]-2-(trifluoromethyl)-3,5,6,7,8,9-hexahydro-4H-cyclohepta[4,5]thieno[2,3-d]pyrimidin-4-one;
tert-butyl 2-(ethyl carbamoylformyl)-5,6,7,8-tetrahydro-4H-cyclohepta[b]thiophene-3-carboxylate;
3-(3-chlorophenyl)-5,6,7,8-tetrahydro[1]benzothieno[2,3-d]pyrimidin-4(3H)-one;
6-(propoxycarbonyl)-2-[(trifluoroacetyl)amino]-4,5,6,7-tetrahydrothieno[2,3-c]pyridine-3-carboxylic acid;
3-(3-bromophenyl)-2-(trifluoromethyl)-3,5,6,7,8,9-hexahydro-4H-cyclohepta[4,5]thieno[2,3-d]pyrimidin-4-one;
2-(trifluoromethyl)-3-[6-(trifluoromethyl)pyridin-3-yl]-3,5,6,7,8,9-hexahydro-4H-cyclohepta[4,5]thieno[2,3-d]pyrimidin-4-one;
3-(5-chloropyridin-2-yl)-5-methyl-2-(trifluoromethyl)-5,6,7,8-tetrahydro[1]benzothieno[2,3-d]pyrimidin-4(3H)-one;
ethyl 2-(2,2,2-trifluoroacetamido)-4,5,6,7,8,9-hexahydrocycloocta[b]thiophene-3-carboxylate;
3-(5-methylpyridin-2-yl)-2-(trifluoromethyl)-5,6,7,8-tetrahydro[1]benzothieno[2,3-d]pyrimidin-4(3H)-one;
3-[3-(methylsulfonyl)phenyl]-2-(trifluoromethyl)-3,5,6,7,8,9-hexahydro-4H-cyclohepta[4,5]thieno[2,3-d]pyrimidin-4-one;
2-(ethyl carbamoylformyl)-5,6,7,8-tetrahydro-4H-cyclohepta[b]thiophene-3-carboxylic acid;
tert-butyl 2-(2,2,2-trifluoroacetamido)-5,7-dihydro-4H-thieno[2,3-c]thiopyran-3-carboxylate;
2-(2,2,2-trifluoroacetamido)-6-acetyl-4,5,6,7-tetrahydrothieno[2,3-c]pyridine-3-carboxylic acid;
3-(4-fluorophenyl)-2-(trifluoromethyl)-3,5,6,7,8,9-hexahydro-4H-cyclohepta[4,5]thieno[2,3-d]pyrimidin-4-one;
N2,N3-dibenzyl-4-bromothiophene-2,3-dicarboxamide;
ethyl 2-(2,2,2-trifluoroacetamido)-5,6,7,8-tetrahydro-4H-cyclohepta[b]thiophene-3-carboxylate;
3-(3,4-dimethylphenyl)-2-(trifluoromethyl)-5,6,7,8-tetrahydro[1]benzothieno[2,3-d]pyrimidin-4(3H)-one;
3-[3-(methylsulfinyl)phenyl]-2-(trifluoromethyl)-3,5,6,7,8,9-hexahydro-4H-cyclohepta[4,5]thieno[2,3-d]pyrimidin-4-one;
3-(3-chlorophenyl)-2-(trifluoromethyl)-3,5,6,7,8,9-hexahydro-4H-cyclohepta[4,5]thieno[2,3-d]pyrimidin-4-one;
2-(2,2,2-trifluoroacetamido)-5,7-dihydro-4H-thieno[2,3-c]thiopyran-3-carboxylic acid;
propyl 3-(3-chlorophenyl)-4-oxo-2-(trifluoromethyl)-3,5,6,8-tetrahydropyrido[4′,3′:4,5]thieno[2,3-d]pyrimidine-7(4H)-carboxylate;
2-[(trifluoroacetyl)amino]-4,7-dihydro-5H-spiro[1-benzothiophene-6,2′-[1,3]dioxolane]-3-carboxylic acid;
N,4,5-triphenylthiophene-3-carboxamide;
isopropyl 5-(4-chlorophenylcarbamoyl)-2-(2,2,2-trifluoroacetamido)-4-methylthiophene-3-carboxylate;
3-(3,4-dichlorophenyl)-2-(trifluoromethyl)-5,6,7,8-tetrahydro[1]benzothieno[2,3-d]pyrimidin-4(3H)-one;
tert-butyl 2-(ethyl carbamoylformyl)-4,5,6,7-tetrahydrobenzo[b]thiophene-3-carboxylate
3-(2,2,2-trifluoroethyl)-2-(trifluoromethyl)-3,5,6,7,8,9-hexahydro-4H-cyclohepta[4,5]thieno[2,3-d]pyrimidin-4-one;
3-tert-butyl 6-propyl 2-amino-4,7-dihydrothieno[2,3-c]pyridine-3,6(5H)-dicarboxylate
7-acetyl-3-(3-chlorophenyl)-2-(trifluoromethyl)-5,6,7,8-tetrahydropyrido[4′,3′:4,5]thieno[2,3-d]pyrimidin-4(3H)-one;
propyl 3-(4-chlorophenyl)-4-oxo-2-(trifluoromethyl)-3,5,6,8-tetrahydropyrido[4′,3′:4,5]thieno[2,3-d]pyrimidine-7(4H)-carboxylate;
2-(2,2,2-trifluoroacetamido)-5,6,7,8-tetrahydro-N-(4-(methylthio)phenyl)-4H-cyclohepta[b]thiophene-3-carboxamide;
3-(4-chlorophenyl)-2-(trifluoromethyl)quinazolin-4(3H)-one;
(Z)-tert-butyl 2-(2,2,2-trifluoroacetamido)-7,8-dihydro-6H-cyclohepta[b]thiophene-3-carboxylate;
ethyl(3-(4-chlorophenylcarbamoyl)-5,6,7,8-tetrahydro-4H-cyclohepta[b]thiophen-2-ylcarbamoyl)formate;
N-(4-chlorophenyl)-2-cyano-2-cycloheptylideneacetamide;
2-(2,2,2-trifluoroacetamido)-N-(4-chlorophenyl)benzamide;
tert-butyl 2-[(methylsulfonyl)amino]-5,6,7,8-tetrahydro-4H-cyclohepta[b]thiophene-3-carboxylate;
3-(4-chlorophenyl)-2-(trifluoromethyl)-5,6,7,8-tetrahydropyrido[4′,3′:4,5]thieno[2,3-d]pyrimidin-4(3H)-one;
2-(2,2,2-trifluoroacetamido)-N-(3-chlorophenyl)-4,5,6,7-tetrahydrobenzo[b]thiophene-3-carboxamide;
2-amino-N-(4-chlorophenyl)-5,6,7,8-tetrahydro-4H-cyclohepta[b]thiophene-3-carboxamide;
2-[(3-{[(4-chlorophenyl)amino]carbonyl}-5,6,7,8-tetrahydro-4H-cyclohepta[b]thien-2-yl)amino]-2-oxoethyl acetate;
(Z)-2-(2,2,2-trifluoroacetamido)-N-(4-chlorophenyl)-7,8-dihydro-6H-cyclohepta[b]thiophene-3-carboxamide;
propyl 3-{[(4-chlorophenyl)amino]carbonyl}-2-[(trifluoroacetyl)amino]-4,7-dihydrothieno[2,3-c]pyridine-6(5H)-carboxylate;
2-(2,2,2-trifluoroacetamido)-4,5,6,7-tetrahydro-N-(5-methylpyridin-2-yl)benzo[b]thiophene-3-carboxamide;
2-(2,2,2-trifluoroacetamido)-N-(4-chlorophenyl)-5,6,7,8-tetrahydro-4H-cyclohepta[b]thiophene-3-carboxamide;
2-acetamido-N-(4-chlorophenyl)-5,6,7,8-tetrahydro-4H-cyclohepta[b]thiophene-3-carboxamide;
2-(2,2,2-trifluoroacetamido)-N-(3-chlorophenyl)-5,7-dihydro-4H-thieno[2,3-c]thiopyran-3-carboxamide;
2-(2,2,2-trifluoroacetamido)-N-(4-chlorophenyl)-4,5,6,7-tetrahydrothieno[2,3-c]pyridine-3-carboxamide;
(3-(4-chlorophenylcarbamoyl)-5,6,7,8-tetrahydro-4H-cyclohepta[b]thiophen-2-ylcarbamoyl)formic acid;
2-(2,2,2-trifluoroacetamido)-N-(4-chlorophenyl)-4,5,6,7-tetrahydro-6-oxobenzo[b]thiophene-3-carboxamide;
2-(2,2,2-trifluoroacetamido)-N-(5-chloropyridin-2-yl)-4,5,6,7-tetrahydrobenzo[b]thiophene-3-carboxamide;
N-(3-chlorophenyl)-2-[(methylsulfonyl)amino]-5,6,7,8-tetrahydro-4H-cyclohepta[b]thiophene-3-carboxamide;
7-acetyl-3-(4-chlorophenyl)-2-(trifluoromethyl)-5,6,7,8-tetrahydropyrido[4′,3′:4,5]thieno[2,3-d]pyrimidin-4(3H)-one;
N-(4-chlorophenyl)-2-[(N,N-dimethylglycyl)amino]-5,6,7,8-tetrahydro-4H-cyclohepta[b]thiophene-3-carboxamide;
2-(trifluoromethyl)-5,6,7,8,9,10-hexahydro-4H-cycloocta[4,5]thieno[2,3-d][1,3]oxazin-4-one;
2-(2,2,3,3,3-pentafluoropropanamido)-N-(4-chlorophenyl)-5,6,7,8-tetrahydro-4H-cyclohepta[b]thiophene-3-carboxamide;
propyl 3-{[(3-chlorophenyl)amino]carbonyl}-2-[(trifluoroacetyl)amino]-4,7-dihydrothieno[2,3-c]pyridine-6(5H)-carboxylate;
2-(2-methoxyacetamido)-N-(4-chlorophenyl)-5,6,7,8-tetrahydro-4H-cyclohepta[b]thiophene-3-carboxamide;
2-(2,2,2-trifluoroacetamido)-6-acetyl-N-(4-chlorophenyl)-4,5,6,7-tetrahydrothieno[2,3-c]pyridine-3-carboxamide;
tert-butyl 2-(2,2,2-trifluoroacetamido)benzoate;
2-(2,2,2-trifluoroacetamido)-6-acetyl-N-(3-chlorophenyl)-4,5,6,7-tetrahydrothieno[2,3-c]pyridine-3-carboxamide;
N-(4-chlorophenyl)-2-(pyruvoylamino)-5,6,7,8-tetrahydro-4H-cyclohepta[b]thiophene-3-carboxamide;
2-(2,2,2-trifluoroacetamido)-N-(3-(trifluoromethyl)phenyl)-5,6,7,8-tetrahydro-4H-cyclohepta[b]thiophene-3-carboxamide;
2-(2,2,2-trifluoroacetamido)benzoic acid;
2-(2,2,2-trifluoroacetamido)-N-(3-chlorophenyl)-5,6,7,8-tetrahydro-4H-cyclohepta[b]thiophene-3-carboxamide;
N-(3-(m-tolylcarbamoyl)-6-tert-butyl-4,5,6,7-tetrahydrobenzo[b]thiophen-2-yl)isonicotinamide;
N-(3-carbamoyl-5-methyl-4-p-tolylthiophen-2-yl)-2-(5-methylfuran-2-yl)quinoline-4-carboxamide;
N-(3-carbamoyl-4-ethyl-5-methylthiophen-2-yl)-7-(trifluoromethyl)-5-phenylpyrazolo[1,5-a]pyrimidine-3-carboxamide;
N-(3-carbamoyl-4-(4-isopropylphenyl)-5-methylthiophen-2-yl)-2-(5-methylfuran-2-yl)quinoline-4-carboxamide;
ethyl 3-(4-(methoxycarbonyl)phenylcarbamoyl)-2-(2-methylbenzamido)-4,5-dihydrothieno[2,3-c]pyridine-6(7H)-carboxylate;
3-(5-chloropyridin-2-yl)-2-(trifluoromethyl)-3,5,6,7,8,9-hexahydro-4H-cyclohepta[4,5]thieno[2,3-d]pyrimidin-4-one;
N-(3-(2-chlorophenylcarbamoyl)-6-tert-butyl-4,5,6,7-tetrahydrobenzo[b]thiophen-2-yl)isonicotinamide;
N-(3-(2-methoxyphenylcarbamoyl)-6-tert-butyl-4,5,6,7-tetrahydrobenzo[b]thiophen-2-yl)nicotinamide;
N,N′-bis[3-(aminocarbonyl)-4,5,6,7-tetrahydro-1-benzothien-2-yl]hexanediamide
3-[4-(methylthio)phenyl]-2-(trifluoromethyl)-5,6,7,8-tetrahydro[1]benzothieno[2,3-d]pyrimidin-4(3H)-one;
ethyl 3-(2,4-dimethoxyphenylcarbamoyl)-2-(benzamido)-4,5-dihydrothieno[2,3-c]pyridine-6(7H)-carboxylate;
ethyl(2E)-3-{5-bromo-2-[hydroxy(oxido)amino]-3-thienyl}acrylate
N-(6-tert-pentyl-3-carbamoyl-4,5,6,7-tetrahydrobenzo[b]thiophen-2-yl)isonicotinamide;
2-(2,2,2-trifluoroacetamido)-N-(5-chloro-2-methoxyphenyl)-5,6,7,8-tetrahydro-4H-cyclohepta[b]thiophene-3-carboxamide;
3-(4-chlorophenyl)-2-(trifluoromethyl)-3,5,6,7,8,9-hexahydro-4H-cyclohepta[4,5]thieno[2,3-d]pyrimidin-4-one;
ethyl 3-(4-methoxyphenylcarbamoyl)-2-(3-fluorobenzamido)-4,5-dihydrothieno[2,3-c]pyridine-6(7H)-carboxylate;
2-[(4-tert-butylbenzoyl)amino]-N-(1,1-dioxidotetrahydro-3-thienyl)-4,5,6,7-tetrahydro-1-benzothiophene-3-carboxamide;
ethyl 3-(2,4-dimethoxyphenylcarbamoyl)-2-(4-chlorobenzamido)-4,5-dihydrothieno[2,3-c]pyridine-6(7H)-carboxylate;
3-(3,4-dichlorophenyl)-2-(trifluoromethyl)-3,5,6,7,8,9-hexahydro-4H-cyclohepta[4,5]thieno[2,3-d]pyrimidin-4-one;
ethyl 3-(4-(methoxycarbonyl)phenylcarbamoyl)-2-(2-phenylacetamido)-4,5-dihydrothieno[2,3-c]pyridine-6(7H)-carboxylate;
ethyl 3-(2,4-dimethoxyphenylcarbamoyl)-4,5-dihydro-2-(nicotinamido)thieno[2,3-c]pyridine-6(7H)-carboxylate;
2-(2-(3-(trifluoromethyl)-4,5,6,7-tetrahydroindazol-1-yl)acetamido)-4,5,6,7-tetrahydrobenzo[b]thiophene-3-carboxamide;
N-(3-carbamoyl-4,5,6,7-tetrahydrobenzo[b]thiophen-2-yl)-7-(difluoromethyl)-5-phenylpyrazolo[1,5-a]pyrimidine-3-carboxamide;
2-(2-(3-(trifluoromethyl)-4,5,6,7-tetrahydroindazol-1-yl)acetamido)-4,5,6,7-tetrahydro-6-methylbenzo[b]thiophene-3-carboxamide;
ethyl 3-(5-chloro-2-methoxyphenylcarbamoyl)-4,5-dihydro-2-(nicotinamido)thieno[2,3-c]pyridine-6(7H)-carboxylate;
5,6,7,8-tetrahydro-2-(thiophene-2-carboxamido)-N-o-tolyl-4H-cyclohepta[b]thiophene-3-carboxamide;
2-(2,2,2-trifluoroacetamido)-N-(3-chlorophenyl)-5,6,7,8-tetrahydro-4H-cyclohepta[b]thiophene-3-carboxamide;
ethyl 3-(4-(methoxycarbonyl)phenylcarbamoyl)-2-(4-fluorobenzamido)-4,5-dihydrothieno[2,3-c]pyridine-6(7H)-carboxylate;
2-(2,2,2-trifluoroacetamido)-5,6,7,8-tetrahydro-N-(naphthalen-1-yl)-4H-cyclohepta[b]thiophene-3-carboxamide;
ethyl 3-(4-methoxyphenylcarbamoyl)-2-(4-chlorobenzamido)-4,5-dihydrothieno[2,3-c]pyridine-6(7H)-carboxylate;
3-{5-[(Z)-(1,5-dioxo-6,7,8,9-tetrahydro-5H-[1]benzothieno[3,2-e][1,3]thiazolo[3,2-a]pyrimidin-2(1H)-ylidene)methyl]-2-furyl}benzoic acid;
2-(5-chloro-2-methoxybenzamido)-5,6,7,8-tetrahydro-4H-cyclohepta[b]thiophene-3-carboxamide;
2-(2,2,2-trifluoroacetamido)-N-(3-chlorophenyl)-5,6,7,8-tetrahydro-4H-cyclohepta[b]thiophene-3-carboxamide; and
N-(3-carbamoyl-4,5,6,7-tetrahydro-6-methylbenzo[b]thiophen-2-yl)-2-(2-methoxyphenyl)quinoline-4-carboxamide.

62. A pharmaceutical composition comprising a compound according to claim 1 and a pharmaceutically acceptable carrier.

63. Use of a compound according to claim 1 for the preparation of a pharmaceutical composition for treating a tumor.

64. The use of claim 63 wherein the tumor is a sarcoma.

65. The use of claim 63 wherein the tumor is a melanoma.

66. The use of claim 63 wherein the tumor is a neuroblastoma.

67. The use of claim 63 wherein the tumor is a carcinoma.

68. The use of claim 63 wherein the tumor is a mesothelioma.
